(19) United States
(12) Patent Application Publication
(10) Pub. No.: US 2011/0257035 A1
(43) Pub. Date: Oct. 20, 2011

(54) IDENTIFICATION OF SIGNATURE GENES ASSOCIATED WITH HEPATOCELLULAR CARCINOMA

(75) Inventor: Carol E. A. Pena, Tarrytown, NY (US)

(73) Assignee: Bayer Healthcare LLC, Tarrytown, NY (US)

(21) Appl. No.: 13/125,212

(22) PCT Filed: Oct. 21, 2009
(86) PCT No.: PCT/US09/61506

§ 371 Date: Jul. 12, 2011

Related U.S. Application Data
(60) Provisional application No. 61/161,315, filed on Mar. 18, 2009.
(60) Provisional application No. 61/160,929, filed on Mar. 17, 2009.
(60) Provisional application No. 61/107,217, filed on Oct. 21, 2008.

(51) Int. Cl. C40B 30/04; C40B 40/06; C12Q 1/02; C40B 40/10; C12Q 1/48; C12Q 1/34

(52) U.S. Cl. 506/9; 435/15; 435/18; 435/29; 506/18; 506/16

(57) ABSTRACT

The present invention relates to, for example, (1) a novel method for identification of clinically useful serum and/or tumor biomarkers and expression signatures that can be used for detection, prognostication and guidance for the treatment of patients with hepatocellular carcinoma (HCC); and (2) discovery of an expression signature that can be used to monitor and/or study the efficacy of a chemotherapeutic regimen, such as, for example, sorafenib (solely or in combination with other agents). The present invention also provides a method for predicting clinical outcomes, such as, for example, overall survival (OS), time to progression (TTP) and/or likelihood of benefitting from a chemotherapeutic treatment (i.e., sorafenib) in HCC patients based on the analysis of such biomarkers.

The present invention relates to, for example, (1) identification of clinically useful serum and/or tumor biomarkers and expression signatures that can be used for detection, prognostication and guidance for the treatment of patients with hepatocellular carcinoma (HCC); and (2) discovery of an expression signature that can be used to monitor and/or study the efficacy of a molecularly targeted agent, such as, for example, sorafenib (solely or in combination with other agents). The present invention also provides a method for predicting clinical outcomes, such as, for example, overall survival (OS), time to progression (TTP) and/or likelihood of benefitting from a therapeutic treatment (i.e., sorafenib) in HCC patients based on the analysis of such biomarkers. Other relevant clinical outcomes include, but are not limited to, progression free survival, time to death, disease free survival, time to symptomatic progression, recurrence free survival, time to recurrence, disease state (i.e., progressive, stable, etc.) and response type (partial, complete, etc.).

### BACKGROUND OF THE INVENTION

Globally, HCC has been identified as the eighth most common cancer, and the most common malignant tumor of males, with an incidence of 1 million new cases each year (Parkin et al., Global cancer statistics, 2002 CA Cancer J. Clin, 55, 74-108, 2005). It is regarded that HCC is responsible for approximately 1 million deaths each year, mainly in underdeveloped and developing countries. In the United States, the 5-year overall survival (1992-1996) rate is reported to be 5%. (El-Serag et al., Hepatology 33:62-65, 2001). Liver dysfunction related to viral infection, e.g., from hepatitis B or C, alcoholic liver damage and alfatoxin B exposure, are reported to generally lead to malignant transformation. Indeed, 80% of HCC worldwide has been reported to be etiologically associated with hepatitis B virus (HBV), and HBV is estimated to account for one in four cases of HCC among non-Asians in the United States (Bosch et al., “Primary liver cancer: worldwide incidence and trends.” Gastroenterology, 127, S5-S16; 2004). According to recent reports, there is no standard therapy for unresectable HCC (Llovet et al., Hepatology. 2003 February; 37(2):429-42). As such, there is a strong need for advancement in prognosis, early detection, and treatment of HCC.

The conventional biomarker for HCC is alpha-fetoproteins (AFP) (Yang et al., “Prospective study of early detection for primary liver cancer.” J Cancer Res Clin Oncol. 1997; 123(6):357-60). However, it has been reported that patients with chronic liver disease and alcoholics also have elevated serum levels of AFP (Mendenhall et al., “Alpha-fetoprotein alterations in alcoholics with liver disease. V.A. Cooperative Study Groups.” Alcohol. 1991; 26(5-6):527-34). Since HCC typically arises in patients with coexisting chronic liver disease, AFP level alone is thought to be a poor biomarker, and has a cancer predictive value only in the 40% range (Huo et al., “The predictive ability of serum alpha-fetoprotein for hepatocellular carcinoma is linked with the characteristics of the target population at surveillance.” J Surg Oncol. 2007 May 25; 95 (8):645-651). Quantitative analysis of isoforms of AFP are thought to improve the diagnostic value to 75%, but is very time consuming, and labor intensive (Sangiovanni et al., Gastroenterology 2004; 126:1005-1014). In addition, about 20% of HCC patients have very low AFP levels, <20 ng/ml. Additional biomarkers such as p53 protein (Raedle et al., Eur J Cancer. 1998 July; 34(8):1198-203) and various aldehyde dehydrogenase isozymes (Park et al., Int J Cancer. 2002 Jan. 10; 97(2):261-5) have been tested. However, none of these have a predictive value that is even as high as AFP (Huo et al.).

Biopsy can be used to diagnose HCC (Walter et al., Curr Opin Gastroenterol. 2008 May; 24(3):312-9. Review), but it is an invasive procedure and, therefore, thought to be less than desirable (Saffroy et al., Clin Chem Lab Med. 2007; 45(9):1169-79. Review). Other diagnostic methods for HCC include ultrasound and computed tomography (CT) scan (Schölmerich et al., Gut 53: 1224-1226; 2004). Only 25-28% of HCC nodules, which are smaller than 2 cm, are reported to be detected by ultrasonography and CT scan during arterial portography.

It would be highly desirable to have biomarkers or a combination of biomarkers that are not only useful in the identification of HCC but also allow characterization of HCC, for example, those that are druggable with sorafenib. The literature on HCC diagnosis has not disclosed heretofore such a biomarker or combination of biomarkers.

### SUMMARY OF THE INVENTION

The present invention provides compositions and methods for cancer diagnostics, and treatment, including, but not limited to, cancer markers. In particular, the present invention provides markers useful in the diagnosis and characterization of patients with hepatocellular carcinoma (HCC) who are to be placed under therapy using a molecularly-targeted agent or standard chemotherapy.

Preferably, the present invention relates to patients with advanced hepatocellular carcinoma (advanced HCC).

The present invention identifies the global changes in gene expression associated with tumors by examining gene expression in plasma (or serum) and other tissues from cancer patients, and from tumor tissue of patients with one outcome versus another, such as following therapy. The present invention also identifies expression profiles which serve as useful diagnostic markers as well as markers that can be used to monitor disease states, disease progression and efficacy of therapeutic intervention.

In one embodiment, the present invention provides for a method for predicting the outcome of a patient suffering from HCC, comprising detecting, in a test sample of said patient, at least one biomarker which is vascular endothelial growth factor (VEGF), soluble VEGF receptor 2 (s-VEGFR-2), soluble VEGF receptor 3 (s-VEGFR-3), soluble c-Kit (s-c-Kit), hepatocyte growth factor (HGF), Ras p 21, phosphorylated ERK (pERK), angiopoietin 2 (Ang2), basic fibroblast growth factor (also known as bFGF, FGF2 or FGF-β; hereinafter bFGF), epidermal growth factor (EGF) and/or insulin-like growth factor 2 (IGF-2) and comparing said levels of said biomarker with a reference standard, wherein differential levels of expression of said biomarker in said test sample compared to said reference standard is indicative of the outcome of HCC. The present invention also provides for a method for predicting the outcome of a patient suffering from HCC, comprising detecting, in a test sample of said patient, a combination of biomarkers, such as, for example, at least one biomarker which is soluble VEGF receptor 3 (s-VEGFR-3), soluble c-Kit (s-c-Kit), hepatocyte growth factor (HGF), Ras p 21 or phosphorylated ERK (pERK) and at least one biomarker which is angiopoietin 2 (Ang2), vascular endothelial growth factor (VEGF), soluble VEGF receptor 2 (s-VEGFR-2), insulin-like growth factor 2 (IGF-2), or basic fibroblast growth factor (bFGF).

The present invention provides methods for detecting differential expression of biomarkers and correlating the expression/levels thereof with reference standards, such as, for example, median values, 75^{th} percentile values, or 25^{th} percentile values, or values defined by a non-HCC population (i.e. healthy subjects, or subjects with liver cirrhosis, hepatitis B virus, and/or hepatitis C virus but without HCC). With respect to the biomarkers of the present invention, such are provided in the table below.

TABLE 1A
Baseline values of HGF, VEGF, sVEGFR3, Ras p21, c-Kit and
sVEGFR2 levels in a patient population.

| Baseline | | | sVEGFR- | | | sVEGFR- |
|---|---|---|---|---|---|---|
| bio- | HGF | VEGF | 3 | Ras p21 | c-Kit | 2 |
| markers | (pg/mL) | (pg/mL) | (pg/mL) | (pg/mL) | (pg/mL) | (pg/mL) |
| 25th | 1802.2 | 32.2 | 30558.9 | 311.9 | 8.0 | 7347.3 |
| % ile | | | | | | |
| Median | 2431.1 | 54.8 | 39587.0 | 776.8 | 11.3 | 8653.4 |
| 75th | 3279.1 | 101.9 | 52314.9 | 1819.7 | 14.4 | 10206.6 |
| % ile | | | | | | |

TABLE 1B
Baseline values of Ang2, bFGF, EGF, and IGF-2 levels in
a patient population.

| Plasma | | | |
|---|---|---|---|
| biomarker | 25th % ile | Median | 75th % ile |
| Ang2 (pg/mL) | 4101.6 | 6061.1 | 9167.4 |
| bFGF (pg/mL) | 3.3 | 7.5 | 17.0 |
| EGF (pg/mL) | 10.3 | 30.4 | 79.5 |
| IGF-2 (ng/mL) | 598.4 | 797.7 | 1078.6 |

TABLE 1C
Detailed chart of biomarker levels.

| Biomarker | | | Ang-2 | Ang-2 | | | bFGFhs | bFGFhs | | |
|---|---|---|---|---|---|---|---|---|---|---|
| Cycle | Ang-2 | Ang-2 | C3 | C3 | bFGFhs | bFGFhs | C3 | C3 | EGF | EGF |
| Unit | 0 | 3 | Change | change | 0 | 3 | change | change | 0 | 3 |
| % ile | pg/ml | pg/ml | % | pg/ml | pg/ml | pg/ml | % | pg/ml | pg/ml | pg/ml |
| 0.05 | 2456.0 | 2342.6 | −38.1 | −4754.5 | 0.5 | 0.6 | −81.8 | −15.0 | 2.2 | 2.7 |
| 0.10 | 3085.4 | 3044.4 | −28.9 | −2509.9 | 1.5 | 1.2 | −73.6 | −10.2 | 3.9 | 3.6 |
| 0.15 | 3376.8 | 3453.8 | −22.8 | −1327.5 | 2.1 | 1.7 | −64.9 | −6.5 | 5.4 | 4.6 |
| 0.20 | 3819.7 | 3659.9 | −19.2 | −931.3 | 2.6 | 2.3 | −53.3 | −4.3 | 7.7 | 6.7 |
| 0.25 | 4101.6 | 4011.9 | −14.0 | −653.7 | 3.3 | 2.8 | −41.0 | −3.3 | 10.3 | 8.6 |
| 0.30 | 4567.1 | 4363.3 | −9.3 | −430.6 | 3.9 | 3.5 | −30.2 | −2.2 | 12.8 | 11.0 |
| 0.35 | 4942.4 | 4731.5 | −5.3 | −208.1 | 4.5 | 4.3 | −23.0 | −1.3 | 17.0 | 13.5 |
| 0.40 | 5210.3 | 5082.1 | −2.1 | −119.0 | 5.4 | 5.2 | −14.4 | −0.6 | 20.7 | 16.7 |
| 0.45 | 5624.9 | 5381.2 | 0.7 | 32.2 | 6.3 | 6.3 | −4.7 | −0.2 | 24.2 | 20.3 |
| 0.50 | 6061.1 | 5775.9 | 5.1 | 220.6 | 7.5 | 7.6 | 4.2 | 0.2 | 30.4 | 27.7 |
| 0.55 | 6588.9 | 6205.5 | 9.0 | 428.4 | 9.5 | 8.7 | 14.5 | 0.6 | 36.2 | 35.2 |
| 0.60 | 6999.0 | 6631.9 | 13.0 | 695.5 | 10.8 | 10.2 | 28.8 | 1.3 | 44.7 | 41.3 |
| 0.65 | 7814.6 | 7528.0 | 18.9 | 1087.5 | 12.9 | 12.2 | 44.8 | 2.3 | 53.8 | 48.5 |
| 0.70 | 8389.2 | 8123.1 | 25.3 | 1426.4 | 15.4 | 15.7 | 66.1 | 3.7 | 66.5 | 60.5 |
| 0.75 | 9167.4 | 9265.9 | 30.8 | 1628.8 | 17.0 | 18.5 | 83.2 | 5.4 | 79.5 | 75.1 |
| 0.80 | 10364.0 | 10329.8 | 41.0 | 2327.8 | 19.3 | 21.5 | 108.2 | 6.8 | 99.8 | 90.7 |
| 0.85 | 11803.5 | 12491.2 | 58.2 | 3019.6 | 24.7 | 26.3 | 132.7 | 10.8 | 128.5 | 118.9 |
| 0.90 | 14016.8 | 15241.2 | 76.0 | 4814.4 | 34.4 | 37.7 | 194.1 | 14.2 | 158.4 | 151.4 |
| 0.95 | 18085.3 | 19795.7 | 126.0 | 8490.5 | 51.2 | 70.9 | 413.5 | 27.9 | 225.5 | 246.2 |

| Biomarker | EGF | EGF | | | IGF-2 | IGF-2 |
|---|---|---|---|---|---|---|
| Cycle | C3 | C3 | IGF-2 | IGF-2 | C3 | C3 |
| Unit | change | change | 0 | 3 | change | change |
| % ile | % | pg/ml | ng/ml | ng/ml | % | ng/ml |
| 0.05 | −87.3 | −103.6 | 415.0 | 353.4 | −46.2 | −497.5 |
| 0.10 | −78.8 | −58.7 | 462.0 | 404.7 | −39.4 | −392.3 |
| 0.15 | −67.9 | −40.8 | 513.3 | 443.8 | −30.7 | −315.4 |
| 0.20 | −63.3 | −33.1 | 546.1 | 476.2 | −26.3 | −250.7 |
| 0.25 | −56.4 | −21.8 | 598.4 | 516.9 | −23.7 | −208.5 |
| 0.30 | −47.2 | −14.1 | 640.8 | 579.0 | −20.5 | −182.5 |
| 0.35 | −38.3 | −11.9 | 671.7 | 628.4 | −18.6 | −156.8 |
| 0.40 | −27.9 | −7.7 | 725.3 | 669.3 | −15.9 | −139.3 |
| 0.45 | −23.2 | −3.9 | 767.6 | 704.1 | −13.9 | −113.3 |
| 0.50 | −14.6 | −2.2 | 797.7 | 737.8 | −11.2 | −94.3 |
| 0.55 | −2.1 | −0.3 | 858.7 | 790.7 | −9.2 | −77.3 |
| 0.60 | 4.0 | 1.2 | 907.7 | 831.5 | −6.3 | −52.9 |
| 0.65 | 14.6 | 3.0 | 959.8 | 863.5 | −4.6 | −28.3 |
| 0.70 | 27.6 | 6.6 | 1015.2 | 915.4 | −0.7 | −5.1 |
| 0.75 | 45.9 | 10.6 | 1078.6 | 1001.5 | 3.4 | 27.9 |
| 0.80 | 87.5 | 19.7 | 1187.6 | 1106.7 | 7.8 | 58.6 |
| 0.85 | 131.8 | 30.1 | 1299.9 | 1216.3 | 13.0 | 94.1 |
| 0.90 | 229.1 | 56.4 | 1517.0 | 1343.5 | 17.3 | 134.5 |
| 0.95 | 417.8 | 93.4 | 1842.6 | 1592.3 | 27.7 | 282.7 |

It is understood that one skilled in the art can utilize art-known techniques for obtaining the structural information of the various biomarkers of the present invention. For example, wherein the biomarker is a gene, such as, for example, Ras p21, one skilled in the art can obtain the structural information of the protein/gene/RNA sequence of such biomarkers via the NCBI's website (available on the world-wide-web at ncbi.nlm.nih.gov). In order to purely facilitate the understanding, a skilled worker will appreciate that Ras p21, as used herein, can relate to members of the Ras family of proteins, such as, for example, H-Ras (GeneID: 3265), K-Ras (GeneID: 3845) and N-Ras (GeneID: 4893).

To further the understanding of the present invention, Ang2, as used herein, can relate to members of angiopoietin family of proteins (for example, Ang2 precursor and Ang 2 splice variant protein described in Kim et al., J. Biol. Chem. 275: 18550-18556, 2000). Preferably, Ang2 refers to the protein having the Uniprot accession No. O15123 (NCBI accession No. NP_{—}001138).

Similarly, bFGF, as used herein, can relate to members of fibroblast growth factor family of proteins (for example, bFGF or FGF2). Preferably, bFGF refers to the human bFGF protein having the Uniprot accession No. P09038 (NCBI accession No. NP_{—}001997).

EGF, as used herein, can relate to members of epidermal growth factor family of proteins (for example, EGF). Preferably, EGF refers to the human EGF protein having the Uniprot accession No. P01133 (NCBI accession No. NP_{—}001954).

IGF2, as used herein, can relate to members of insulin-like growth factor family of proteins (for example, IGF1, IGF2). Preferably, IGF2 refers to the human IGF2 protein having the Uniprot accession No. P01344 (NCBI accession No. NM_{—}000612).

With respect to the several isoforms of VEGF that are known in the art, the invention provides for a method for predicting the outcome of a patient suffering from HCC, comprising detecting, in a test sample of said patient, at least one biomarker which is vascular endothelial growth factor-A (VEGF-A; GeneID: 7422).

Additionally, the skilled worker will appreciate that owing to the correlation between the levels of circulating (i.e., soluble forms) biomarker proteins and native forms thereof, the present invention is not limited to circulating forms of the biomarker proteins, although such are preferred. For example, as described hereinbefore, the present invention provides for a method for predicting the outcome of a patient suffering from HCC, comprising detecting, in a test sample of said patient, at least one biomarker which is extracellular domain (ECD) of c-Kit (“soluble” c-KIT). Since circulating c-Kit ECD is released from the tumor itself, it may reflect the level of c-Kit present in the tumor. As such, the present invention is not limited to biomarkers such as, for example, circulating ECD of c-Kit (s-c-Kit), but also includes full-length c-Kit on the tumor.

Preferably, the biomarkers of the present invention are found or detectable in plasma and are hence referred as plasma biomarkers.

The inventors of the present invention have discovered that plasma levels of VEGF, s-VEGFR-3, IGF-2, and Ang2, either solely or in combination, are good prognostic indicators of overall survival (OS) in patients with HCC. VEGF and Ang2 have been found to be a good prognostic indicator of time to progression (TTP) and overall survival (OS). IGF-2 has been found to be a good indicator of OS. Other relevant clinical outcomes include, but are not limited to, progression free survival, time to death, disease free survival, time to symptomatic progression, recurrence free survival, time to recurrence, disease state (i.e., progressive, stable, etc.) and response type (partial, complete, etc.)

In one embodiment of the invention, the inventors have identified that low levels of plasma VEGF and/or low levels of plasma Ang2 are associated with improved overall survival (OS) in HCC patients. In studies related to this embodiment, a 75^{th} percentile plasma VEGF level in HCC patients (101.928 pg/ml for VEGF) was used as a reference standard for characterization of patients with “low” or “high” plasma biomarker (i.e., VEGF, etc.) levels. With respect to Ang2, the median (50^{th} percentile) plasma Ang2 level (6.0611 ng/ml) was used as a reference standard for characterization of patients with “low” or “high” plasma biomarker levels. In such studies, it was identified that patients with higher than 6.061 ng/ml plasma Ang2 levels had poorer overall survival than patients whose plasma Ang2 level was lower than 6.061 ng/ml. The association between plasma Ang2 levels and OS was found to be statistically significant.

In a related embodiment, the inventors have identified that high levels of plasma IGF-2 is associated with improved overall survival (OS) in HCC patients. In studies related to this embodiment, a median (50^{th} percentile) plasma IGF-2 level (797.7 ng/ml) was used as a reference standard for characterization of patients with “low” or “high” plasma biomarker levels. It was identified herein that patients with higher than 797.7 ng/ml plasma IGF-2 levels had improved overall survival than patients whose plasma IGF-2 levels were lower than 797.7 ng/ml. The association between plasma IGF-2 levels and OS was found to be statistically significant.

Another aspect of the present invention relates to association of plasma biomarkers with time to progression. In such studies, it was identified that patients with higher than 6.061 ng/ml plasma Ang2 levels had shorter time to progression than patients whose plasma Ang2 level was lower than 6.061 ng/ml. The association between plasma Ang2 levels and TTP was found to be statistically significant.

The association between plasma VEGF and plasma Ang2 levels and independently-assessed time to progression (TTP) in HCC patients was found to be similar (i.e., low VEGF and/or low Ang2 levels being associated with increased TTP).

The association between plasma s-VEGFR-3 levels and overall survival in patients with HCC was found to be statistically significant, with low plasma s-VEGFR-3 levels being associated with improved overall survival. In studies related to these embodiments, a 25^{th} percentile plasma s-VEGFR-3 levels in HCC patients (30.559 ng/ml) was used as a reference standard for determination of “low” vs. “high” s-VEGFR-3 levels.

In another embodiment of the invention, the inventors have identified that low levels of Ras p21 biomarker are associated with time to progression (TTP). In such embodiments, a median level of Ras p21 (1042.9 pg/mL) was used as a reference standard for characterization of patients with “low” or “high” Ras p21. Lower levels of Ras p21 associated with shorter time to progression (TTP)—in a multivariate analysis of placebo patients. So, the present invention provides identification of Ras p21 as a prognostic factor for TTP, wherein untreated patients with low levels of Ras p21 have worse TTP outcome than those with high levels.

The association between plasma Ras p21 levels and time to progression in patients with HCC was found to be statistically significant, with high plasma Ras p21 levels being associated with improved time to progression in placebo patients. In studies related to these embodiments, a lower plasma Ras 21 level was associated with a significantly increased risk of progression. For example, a patient with Ras p21 at the 25th percentile of advanced HCC patients (464.9 pg/mL) has a 29.4% greater risk of progression than a patient at the 75th percentile.

The present invention therefore allows prognostication of outcome of patients diagnosed with HCC, for example, prediction of overall survival (OS) or time to progression (TTP), based on the levels of one or more of the aforementioned plasma biomarkers. The method comprises detecting, in a test sample of said patient, at least one biomarker which is vascular endothelial growth factor (VEGF), soluble VEGF receptor-3 (s-VEGFR-3), Ras p21, hepatocyte growth factor (HGF), angiopoietin 2 (Ang2), basic fibroblast growth factor (bFGF), epidermal growth factor (EGF) and/or insulin-like growth factor 2 (IGF-2) and comparing said level of expression of said biomarker in said patient test sample with a reference standard, wherein differential levels of expression of said biomarker in said test sample compared to said reference standard is indicative of said outcome. Preferably, the biomarker is a plasma biomarker such as VEGF, s-VEGFR-3, Ang2, IGF-2 or a combination thereof.

In one embodiment, the outcome is OS and the method comprises detecting at least one plasma biomarker which is Ang2, IGF2, VEGF or s-VEGFR-3. As stated hereinbefore, low level of plasma Ang2, plasma VEGF or plasma s-VEGFR-3 (compared to a reference standard, for example, 50^{th} percentile plasma Ang2/IGF-2 levels, 75^{th} percentile plasma VEGF levels or 25^{th} percentile plasma s-VEGFR-3 levels in an HCC patient population) is associated with improved OS in HCC patients.

In another embodiment, the outcome is OS and the method comprises detecting at least one plasma biomarker which is Ang2, IGF-2 or s-VEGFR-3 and optionally VEGF. As stated hereinbefore, low level of plasma HGF, plasma VEGF or plasma s-VEGFR-3 (compared to a reference standard, for example, 75^{th} percentile plasma HGF/VEGF levels or 25^{th} percentile plasma s-VEGFR-3 levels in an HCC patient population) is associated with improved OS in HCC patients.

In a related embodiment, the outcome is time to progression (TTP) and the method comprises detecting at least one biomarker which is Ang2, Ras p21, VEGF or a combination thereof. Preferably the biomarker is a plasma biomarker such as Ang2 or VEGF. As stated hereinbefore, low levels of plasma Ang2 in an HCC patient (compared to a reference standard, for example, 50^{th} percentile plasma Ang2 levels in an HCC patient population) is associated with improved TTP. Similarly, low levels of plasma VEGF in an HCC patient (compared to a reference standard, for example, 75^{th} percentile plasma VEGF levels in an HCC patient population) is associated with improved TTP.

The present invention also allows for the prognostication of outcome, for example, overall survival and/or time to progression in a patient suffering from HCC comprising detecting a combination of the aforementioned biomarkers. Preferred combinations include, but are not included to, HGF and VEGF; HGF and s-VEGFR-3; VEGF and s-VEGFR-3; HGF, VEGF and s-VEGFR-3; HGF and Ras p21; HGF, VEGF and Ras p21; VEGF and Ras p21; s-VEGFR-3 and Ras p21; c-KIT and bFGF; c-KIT and IGF-2; bFGF and IGF-2; HGF and bFGF; HGF and IGF-2, etc.

Particularly preferred combinations include, but are not included to, c-KIT and bFGF; c-KIT and IGF-2; bFGF and IGF-2; HGF and bFGF; HGF and IGF-2, etc.

A skilled artisan will appreciate that owing to the higher predictive power of a combination of biomarkers, the use of a combination of biomarkers (or proteomic signatures) such as ones described hereinbefore, are particularly preferred.

Merely for illustrative purposes, the inventors of the present application have identified that:

(a) HCC patients with high BL VEGF have shorter OS than patients with low BL VEGF (An HCC patient at the 75^{th} percentile has a greater risk of death than an HCC patient at the 25^{th} percentile);
(b) HCC patients with high BL s-VEGFR-3 have shorter OS than HCC patients with low BL s-VEGFR-3 (An HCC patient at the 75^{th} percentile has a greater risk of death than an HCC patient at the 25^{th} percentile);
(c) HCC patients with high baseline (BL) Ang2 have shorter OS than those with low BL Ang2 (An HCC patient at the 75^{th} percentile has a greater risk of death than an HCC patient at the 25^{th} percentile);
(d) HCC patients with high baseline (BL) IGF-2 have longer OS than those with low BL IGF-2 (An HCC patient at the 75^{th} percentile has a lower risk of death than an HCC patient at the 25^{th} percentile);
(e) HCC patients with low baseline Ras p21 have shorter time to progression (TTP) than HCC patients with high Ras p21 levels (An HCC patient at the 25^{th} percentile has a greater risk of progression than an HCC patient at the 75^{th} percentile);
(f) HCC patients with high BL VEGF have shorter TTP than patients with low BL VEGF (An HCC patient at the 75^{th} percentile has a greater risk of progression than a patient at the 25^{th} percentile);
(g) HCC patients with high baseline (BL) Ang2 have shorter TTP than those with low BL Ang2 (An HCC patient at the 75^{th} percentile has a greater risk of death than an HCC patient at the 25^{th} percentile).

TABLE 2
Examples of baseline plasma biomarkers as prognostic factors for HCC
(p ≦ 0.05 indicates significance)

| | | | | | | | | Corr. with |
|---|---|---|---|---|---|---|---|---|
| | | Biomarker | | | | | | (longer/ |
| Bio- | | variable | Analysis | Pop. | | | | shorter) |
| marker | TP | type | performed | used | EP | p-value | (B) | OS/TTP |
| c-Kit | Baseline | Continuous | Multivariate | All patients | OS | 0.078 | − | Longer |
| c-Kit | Baseline | Continuous | Multivariate | Sorafenib | OS | 0.051 | − | Longer |
| | | | | pts only | | | | |
| c-Kit | Baseline | Binned | Multivariate | All patients | OS | 0.033 | − | Longer |
| c-Kit | Baseline | Binned | Multivariate | Sorafenib | OS | 0.033 | − | Longer |
| | | | | pts only | | | | |
| HGF | Baseline | Continuous | Univariate | Placebo pts | OS | 0.013 | + | Shorter |
| | | | | only | | | | |
| HGF | Baseline | Continuous | Multivariate | Sorafenib | OS | 0.004 | + | Shorter |
| | | | | pts only | | | | |
| HGF | Baseline | Continuous | Multivariate | Sorafenib | TTP | 0.081 | + | Shorter |
| | | | | pts only | | | | |
| HGF | Baseline | Binned | Univariate | Placebo pts | OS | 0.032 | + | Shorter |
| | | | | only | | | | |
| HGF | Baseline | Binned | Multivariate | Sorafenib | OS | 0.017 | + | Shorter |
| | | | | pts only | | | | |
| Ras p21 | Baseline | Continuous | Mutivariate | Placebo pts | OS | 0.075 | − | Longer |
| | | | | only | | | | |
| Ras p21 | Baseline | Continuous | Mutivariate | Placebo pts | TTP | 0.011 | − | Longer |
| | | | | only | | | | |
| Ras p21 | Baseline | Binned | Mutivariate | Placebo pts | TTP | 0.001 | − | Longer |
| | | | | only | | | | |
| VEGF | Baseline | Continuous | Univariate | Placebo pts | OS | 0.001 | + | Shorter |
| | | | | only | | | | |
| VEGF | Baseline | Continuous | Multivariate | All patients | OS | 0.002 | + | Shorter |
| VEGF | Baseline | Continuous | Multivariate | All patients | TTP | 0.081 | + | Shorter |
| VEGF | Baseline | Continuous | Multivariate | Placebo pts | OS | 0.001 | + | Shorter |
| | | | | only | | | | |
| VEGF | Baseline | Continuous | Multivariate | Placebo pts | TTP | 0.005 | + | Shorter |
| | | | | only | | | | |
| VEGF | Baseline | Binned | Univariate | Placebo pts | OS | 0.001 | + | Shorter |
| | | | | only | | | | |
| VEGF | Baseline | Binned | Multivariate | All patients | OS | 0.006 | + | Shorter |
| VEGF | Baseline | Binned | Multivariate | Placebo pts | OS | 0.001 | + | Shorter |
| | | | | only | | | | |
| VEGF | Baseline | Binned | Multivariate | Placebo pts | TTP | <0.001 | + | Shorter |
| | | | | only | | | | |
| VEGFR-3 | Baseline | Continuous | Univariate | Placebo pts | OS | 0.014 | + | Shorter |
| | | | | only | | | | |
| VEGFR-3 | Baseline | Binned | Univariate | Placebo pts | OS | 0.083 | + | Shorter |
| | | | | only | | | | |

*Multivariate analyses performed using Cox proportional hazard models.
For OS analyses, variables included (all baseline values):
treatment group (when both groups included), 8 clinical factors shown to be prognostic for OS in SHARP trial (ECOG PS, tumor burden, AFP, macroscopic vascular invasion, Child-Pugh status, albumin, alkaline phosphatase, total bilirubin), and 6 baseline plasma biomarkers (c-KIT, HGF, Ras p21, VEGF, VEGFR-2, VEGFR-3).
For TTP analyses, variables included (all baseline values):
treatment group (when both groups included), 4 clinical factors shown to be prognostic for TTP in SHARP trial (tumor burden, AFP, alkaline phosphatase, etiology), and 6 baseline plasma biomarkers (c-KIT, HGF, Ras p21, VEGF, VEGFR-2, VEGFR-3).

The above baseline plasma biomarkers were obtained for the patient class investigated. It is foreseen that baseline values may vary for a patient class and the invention is not limited to the use of these baseline values.

In the present invention, there is also provided a method for prognosticating outcome of a patient suffering from HCC, comprising detecting in a test sample of said patient, at least one biomarker which is HGF, VEGF, s-VEGFR-3, c-Kit, Ang2 or IGF-2, preferably s-VEGFR-3, Ang2, or IGF-2 and particularly preferably s-VEGFR-3 and Ang2, and optionally at least one additional parameter which is

(a) Eastern Cooperative Oncology Group performance status (ECOG PS: 0 versus 1+2),

(b) macrovascular vascular invasion;

(c) tumor burden;

(d) extra-hepatic spread;

(e) levels of alpha fetoprotein (AFP);

(f) levels of alkaline phosphatase (AP);

(g) ascites;

(h) levels of bilirubin;

(i) levels of albumin;

(j) PT score; and/or

(k) child-pugh score.

The skilled artisan will readily appreciate that the biomarkers of the present invention provide prognostic information are valuable independently of the aforementioned additional clinical prognostic factors, which are understood in the art. For example, patients with high ECOG score do worse than patients with low ECOG score. As such, the biomarkers of the present invention provide better prognostic information than just ECOG score (and the other 7 known prognostic factors for HCC). So, for prognostic determination, one skilled in the art can use any or all (or any combination) of these prognostic factors plus the prognostic biomarkers to determine an individual patient's prognosis.

The additional clinical prognostic factors of the present invention are:

ECOG (Eastern Cooperative Oncology Group) performance status—A measure of what the patient is capable of doing. Evaluated on a scale of 0 to 5. In the methods of the present invention, only patients with 0 to 2 ECOG status were enrolled, most patients were 0 or 1 (very few 2s). For the statistical analyses, patients were divided into 2 groups: 0 vs. 1 or 2 (Oken et al., American Journal of Clinical Oncology, 1982). Scale is provided in table below.

TABLE 3
ECOG performance status.
ECOG PERFORMANCE STATUS\grave{ }

| Grade | ECOG |
|---|---|
| 0 | Fully active, able to carry on all pre-disease performance |
| | without restriction |
| 1 | Restricted in physically strenuous activity but ambulatory and |
| | able to carry out work of a light of sedentary nature, e.g. light |
| | house work, office work |
| 2 | Ambulatory and capable of all selfcare but unable to carry out |
| | any work activities. Up and about more than 50% of waking |
| | hours |
| 3 | Capable of only limited selfcare, contined to bed or chair more |
| | than 50% of waking hours |
| 4 | Completely disabled Cannot carry on any selfcare. Totally |
| | contined to bed or chair |
| 5 | Dead |

\grave{ }As published in Am J Clin. Oncol Okers, M. M., Crench, R H, Tomey, S C., Horton, J., Davis, T. E., McFadden, E. T. Carbone. P. P.: Toxicity And Response Onterio Of The Eastern Cooperative Oncology Group. Am J Clin Oncol 5: 649-655, 1982.

Tumor burden—The presence of “tumor burden” indicates that either the tumor has vascular invasion, extrahepatic spread, or both. This is a yes or no variable, where yes is indicative of worse outcome.

AFP—High AFP levels are indicative of worse prognosis. In the present invention (and in the published analysis from the SHARP study showing the prognostic value of AFP; reference: Llovet et al. 2008 Sorafenib in advanced hepatocellular carcinoma. NEJM 359(4):378-390) median values were used to classify patients as having “high” vs. “low” levels of additional parameters, such as, for example, AFP levels. Further analyses were performed using other AFP cutoffs that have been published as clinically significant, such as, for example, 100, 200 and 400 ng/mL. No matter how “high” vs. “low” AFP levels was defined, it remained a significant prognostic factor for both OS and TTP. More importantly, using different cutoffs for AFP did not affect the significance of the biomarker findings.

Macroscopic vascular invasion—This additional parameter is evaluated on a binary (i.e., yes or no) scale, wherein yes (presence of vascular invasion) correlates with poor outcome.

Child-Pugh score—Scored as A, B or C, where “higher” score (C) indicates poorer outcome. A, B and C are defined by the clinical measurements shown in the table below.

TABLE 2
Child-Pugh classification of liver disease severity

| Measure | 1 Point | 2 Points | 3 Points |
|---|---|---|---|
| Bilirubin (mg/dl) | <2 | 2-3 | >3 |
| Albumin (g/dl) | >3.5 | 2.8-3.5 | <2.8 |
| Prothrombin time | 1-3 | 4-6 | >6 |
| Ascites | None | Slight | Moderate |
| Encephalopathy (grade) | None | I-II | III-IV |

Grade A, 5-6 points;
grade B, 7-9 points;
grade C, 10-15 points.

Albumin, alkaline phosphatase, and total bilirubin—In the present analyses (and in the published analysis from the SHARP study showing the prognostic value of these 3 factors) a median value of albumin, AP and bilirubin were used to separate high from low levels. For albumin lower levels associate with worse outcome. For alkaline phosphatase and total bilirubin higher levels associate with worse outcome.

The additional parameter can be determined by art known techniques, for example, median levels of AFP, presence/absence of macrovascular invasion, and median levels of bilirubin, albumin, and/or AP. Other values, such as, for example, 100 ng/mL, 200 ng/mL, or 400 ng/mL of AFP may also be used to define high versus low AFP since the biomarker results hold true no matter which is used (for example, median AFP can be used). The level of additional parameter may be determined by the attending physician (for example, macrovascular invasion or tumor burden) or determined by a clinician (for example, plasma bilirubin, albumin, AFP, and AP levels). The grading of the additional parameter as “high” or “low” may be done using routine scoring procedures. For example, a binary scoring technique (i.e., 1=above median, 0=below median), a scale system (i.e., scale of 1-5, wherein 1 is lowest and 5 is the highest) or actual values may be employed.

As a representative example, the directionality of the association of these parameters with HGF is shown in Table 4 below.

The clinical parameters in Table 4 were obtained for the patient class investigated. It is foreseen that baseline values may vary for a patient class and the invention is not limited to the use of these values.

In an embodiment, the present invention provides for prognostication of overall survival of a patient suffering from HCC, comprising

detecting in a test sample of said patient, at least one biomarker which is plasma Ang2 and at least one additional parameter which is

(a) Eastern Cooperative Oncology Group performance status (ECOG PS: 0 versus 1+2),

(b) macrovascular vascular invasion;

(c) tumor burden;

(d) extra-hepatic spread;

(e) levels of alpha fetoprotein (AFP);

(f) levels of alkaline phosphatase (AP);

(g) ascites;

(h) levels of bilirubin;

(i) levels of albumin;

(j) PT score; and/or

(k) child-pugh score;

and comparing said plasma HGF levels and said additional parameter in said patient with

a reference standard; wherein

high levels of said plasma Ang2 levels combined with low levels of the additional parameter (i) or high levels of the additional parameter which is parameters (a)-(h) or parameter (j)-(k), is indicative of poor overall survival.

TABLE 4
Baseline HGF levels and demographic variables.
(p ≦ 0.05 indicates significance)

| | | HGF level | | |
|---|---|---|---|---|
| Demographic variable at baseline | N | Mean | Median | P-value* |

SEX

| | | | | |
|---|---|---|---|---|
| MALE | 403 | 2772.2 | 2429.6 | 0.833 |
| FEMALE | 64 | 2754.2 | 2530.6 | |

AGE GROUP

| | | | | |
|---|---|---|---|---|
| <65 YRS | 185 | 2768.5 | 2480.7 | 0.931 |
| >=65 YRS | 282 | 2770.6 | 2429.3 | |

ECOG STATUS

| | | | | |
|---|---|---|---|---|
| 0 | 241 | 2778.6 | 2418.7 | 0.936 |
| >0 | 226 | 2760.3 | 2503.5 | |

TUMOR BURDEN

| | | | | |
|---|---|---|---|---|
| ABSENT | 146 | 2635.4 | 2429.3 | 0.298 |
| PRESENT | 321 | 2830.9 | 2464.3 | |

AFP

| | | | | |
|---|---|---|---|---|
| <=MEDIAN | 233 | 2549.5 | 2301.8 | 0.011 |
| >MEDIAN | 234 | 2989.0 | 2570.1 | |

MACROSCOPIC VASCULAR INVASION

| | | | | |
|---|---|---|---|---|
| NO | 290 | 2570.4 | 2304.5 | 0.002 |
| YES | 177 | 3096.4 | 2663.7 | |

Albumin

| | | | | |
|---|---|---|---|---|
| <=MEDIAN | 232 | 3145.5 | 2808.5 | <0.001 |
| >MEDIAN | 235 | 2398.8 | 2066.6 | |

Alkaline Phosphatase

| | | | | |
|---|---|---|---|---|
| <=MEDIAN | 235 | 2472.1 | 2210.0 | <0.001 |
| >MEDIAN | 232 | 3071.2 | 2662.6 | |

Total Bilirubin

| | | | | |
|---|---|---|---|---|
| <=MEDIAN | 232 | 2412.8 | 2154.4 | <0.001 |
| >MEDIAN | 235 | 3122.2 | 2738.8 | |

TABLE 4B
Baseline Ang2 levels and demographic variables.
(p ≦ 0.05 indicates significance)

| | | Ang2 level | | |
|---|---|---|---|---|
| Demographic variable at baseline | N | Mean | Median | P-value* |

SEX

| | | | | |
|---|---|---|---|---|
| MALE | 405 | 7672.5 | 6018.8 | 0.6853 |
| FEMALE | 64 | 8175.0 | 6264.0 | |

AGE GROUP

| | | | | |
|---|---|---|---|---|
| <65 YRS | 187 | 7850.3 | 6422.8 | 0.6796 |
| >=65 YRS | 282 | 7668.6 | 5945.0 | |

ECOG STATUS

| | | | | |
|---|---|---|---|---|
| 0 | 239 | 6608.8 | 5598.4 | <0.0001 |
| >0 | 230 | 8917.6 | 6950.4 | |

TUMOR BURDEN

| | | | | |
|---|---|---|---|---|
| ABSENT | 145 | 6800.0 | 5442.8 | 0.018 |
| PRESENT | 324 | 8162.2 | 6348.2 | |

AFP

| | | | | |
|---|---|---|---|---|
| <=MEDIAN | 234 | 6887.8 | 5402.3 | 0.0015 |
| >MEDIAN | 235 | 8590.7 | 6546.8 | |

MACROSCOPIC VASCULAR INVASION

| | | | | |
|---|---|---|---|---|
| NO | 291 | 6889.6 | 5514.3 | <0.0001 |
| YES | 178 | 9133.1 | 7357.6 | |

Albumin

| | | | | |
|---|---|---|---|---|
| <=MEDIAN | 236 | 9273.2 | 7475.7 | <0.0001 |
| >MEDIAN | 233 | 6189.2 | 4979.1 | |

Alkaline Phosphatase

| | | | | |
|---|---|---|---|---|
| <=MEDIAN | 236 | 6305.7 | 5112.2 | <0.0001 |
| >MEDIAN | 233 | 9195.0 | 7288.1 | |

Total Bilirubin

| | | | | |
|---|---|---|---|---|
| <=MEDIAN | 231 | 6857.3 | 5307.6 | 0.0003 |
| >MEDIAN | 238 | 8598.9 | 6877.5 | |

In particular, the inventors have found that high Ang2 levels associated with:
High ECOG score (>0)
Presence of “tumor burden” (MVI or EHS)
High AFP (>median)
Presence of macroscopic vascular invasion
Low albumin (<=median)
High alkaline phosphatase (>median)
High total bilirubin (>median)
Directionality of all these associations was very consistent.

TABLE 4C
Baseline IGF-2 levels and demographic variables.
(p ≦ 0.05 indicates significance)

| | | IGF-2 level | | |
|---|---|---|---|---|
| Demographic variable at baseline | N | Mean | Median | P-value* |

SEX

| | | | | |
|---|---|---|---|---|
| MALE | 408 | 840.7 | 778.1 | <0.0001 |
| FEMALE | 64 | 1374.7 | 1213.0 | |

AGE GROUP

| | | | | |
|---|---|---|---|---|
| <65 YRS | 188 | 972.7 | 854.8 | 0.0182 |
| >=65 YRS | 284 | 873.6 | 793.2 | |

ECOG STATUS

| | | | | |
|---|---|---|---|---|
| 0 | 241 | 935.4 | 855.8 | 0.2197 |
| >0 | 231 | 889.9 | 777.5 | |

TUMOR BURDEN

| | | | | |
|---|---|---|---|---|
| ABSENT | 146 | 885.2 | 780.1 | 0.4098 |
| PRESENT | 326 | 925.6 | 838.3 | |

AFP

| | | | | |
|---|---|---|---|---|
| <=MEDIAN | 235 | 947.2 | 847.2 | 0.1107 |
| >MEDIAN | 237 | 879.2 | 788.9 | |

MACROSCOPIC VASCULAR INVASION

| | | | | |
|---|---|---|---|---|
| NO | 293 | 960.8 | 871.8 | 0.001 |
| YES | 179 | 835.1 | 744.4 | |

Albumin

| | | | | |
|---|---|---|---|---|
| <=MEDIAN | 237 | 754.6 | 653.8 | <0.0001 |
| >MEDIAN | 235 | 1073.0 | 948.9 | |

Alkaline Phosphatase

| | | | | |
|---|---|---|---|---|
| <=MEDIAN | 239 | 929.6 | 856.6 | 0.1715 |
| >MEDIAN | 233 | 896.2 | 754.7 | |

Total Bilirubin

| | | | | |
|---|---|---|---|---|
| <=MEDIAN | 234 | 1056.9 | 938.9 | <0.0001 |
| >MEDIAN | 238 | 771.7 | 683.0 | |

In particular, the inventors have found that low IGF-2 levels associated with:
Male gender
Age >=65 years
Presence of macroscopic vascular invasion
Low albumin (<=median)
High total bilirubin (>median)

The present invention also prognosticates the outcome of HCC patients using biomarkers as bifurcated variables. Results of this study are presented in Tables 4D and 4E.

TABLE 4D
Multivariate analysis to identify factors independently
prognostic for OS in HCC - using biomarkers as bifurcated
variables (p ≦ 0.05 indicates significance)

| | P-value | | |
|---|---|---|---|
| | | Placebo pts | Soraf pts |
| Variable | All pts | only | only |
| Treatment | 0.029 | | |
| ECOG PS (0 vs 1 + 2) | 0.007 | 0.235 | 0.060 |
| Tumor burden | 0.015 | 0.981 | <0.001 |
| Baseline AFP | 0.001 | 0.005 | 0.067 |
| Macroscopic vascular invasion | 0.005 | 0.001 | 0.774 |
| Child-Pugh status | 0.226 | <0.0001 | 0.835 |
| Baseline albumin | 0.030 | 0.036 | 0.336 |
| Baseline alkaline phosphatase | 0.009 | 0.146 | 0.004 |
| Baseline total bilirubin | 0.001 | 0.023 | 0.011 |
| s-c-KIT | 0.020 | 0.562 | 0.003 |
| HGF | 0.475 | 0.594 | 0.064 |
| Ras p21 | 0.957 | 0.949 | 0.927 |
| VEGF | 0.006 | 0.001 | 0.804 |
| sVEGFR-2 | 0.970 | 0.990 | 0.784 |
| sVEGFR-3 | 0.751 | 0.211 | 0.390 |
| Ang2 | 0.004 | 0.021 | 0.021 |
| bFGF | 0.189 | 0.244 | 0.423 |
| EGF | 0.308 | 0.206 | 0.816 |
| IGF-2 | 0.280 | 0.617 | 0.372 |

TABLE 4E
New multivariate analysis to identify factors independently
prognostic for OS in HCC - using biomarkers as bifurcated
variables (p ≦ 0.05 indicates significance)

| | P-value | | |
|---|---|---|---|
| | | Placebo pts | Soraf pts |
| Variable | All pts | only | only |
| Treatment | 0.043 | | |
| ECOG PS (0 vs 1 + 2) | 0.018 | 0.290 | 0.070 |
| Baseline AFP | 0.001 | 0.008 | 0.014 |
| Macroscopic vascular invasion | <0.0001 | <0.001 | 0.005 |
| Extrahepatic spread | 0.231 | 0.851 | 0.016 |
| Baseline alkaline phosphatase | <0.001 | 0.015 | <0.001 |
| Ascites | 0.053 | 0.072 | 0.527 |
| Bilirubin score | 0.922 | 0.763 | 0.123 |
| Albumin score | 0.794 | 0.173 | 0.681 |
| PT score | 0.614 | 0.193 | 0.922 |
| s-c-KIT | 0.101 | 0.888 | 0.006 |
| HGF | 0.215 | 0.496 | 0.014 |
| Ras p21 | 0.620 | 0.546 | 0.998 |
| VEGF | 0.017 | 0.002 | 0.956 |
| sVEGFR-2 | 0.863 | 0.831 | 0.656 |
| sVEGFR-3 | 0.972 | 0.295 | 0.482 |
| Ang2 | 0.002 | 0.002 | 0.091 |
| bFGF | 0.457 | 0.646 | 0.530 |
| EGF | 0.420 | 0.429 | 0.674 |
| IGF2 | 0.846 | 0.788 | 0.544 |

TABLE 4F
Summary of new multivariate analysis to identify factors independently prognostic for
OS in HCC, the results of which are presented in FIG. 4E. Hazard ratios (HR) were
calcualted for each parameter studied. (p ≦ 0.05 indicates significance)

| | Multivariate Analysis of | | Multivariate Analysis of | | Multivariate Analysis of | |
|---|---|---|---|---|---|---|
| | All Patients | | Placebo Patients | | Sorafenib Patients | |
| | P-value | HR | P-Value | HR | P-value | HR |
| Treatment | 0.043 | 0.779 | | | | |
| ECOG PS (0 vs 1 + 2) | 0.018 | 1.353 | 0.290 | 1.203 | 0.070 | 1.451 |
| Baseline AFP | 0.001 | 1.489 | 0.008 | 1.575 | 0.014 | 1.574 |
| Macroscopic vascular invasion | <0.001 | 1.806 | <0.001 | 1.885 | 0.005 | 1.725 |
| Extrahepatic spread | 0.231 | 1.161 | 0.851 | 0.968 | 0.016 | 1.599 |
| Baseline alkaline phosphatase | <0.001 | 1.602 | 0.015 | 1.548 | <0.001 | 1.872 |
| Ascites | 0.053 | 1.384 | 0.072 | 1.529 | 0.527 | 1.190 |
| Bilirubin score | 0.922 | 1.027 | 0.763 | 0.893 | 0.123 | 1.914 |
| Albumin score | 0.794 | 1.055 | 0.173 | 1.511 | 0.681 | 0.884 |
| PT score | 0.614 | 1.151 | 0.193 | 1.868 | 0.922 | 0.961 |
| s-c-KIT | 0.101 | 0.814 | 0.888 | 1.025 | 0.006 | 0.581 |
| HGF | 0.215 | 1.200 | 0.496 | 0.863 | 0.014 | 1.718 |
| Ras p21 | 0.620 | 0.913 | 0.546 | 0.857 | 0.998 | 0.999 |
| VEGF | 0.017 | 1.470 | 0.002 | 1.969 | 0.956 | 1.014 |
| sVEGFR-2 | 0.863 | 0.974 | 0.831 | 1.047 | 0.656 | 1.103 |
| sVEGFR-3 | 0.972 | 0.995 | 0.295 | 0.832 | 0.482 | 1.157 |
| Ang2 | 0.002 | 1.545 | 0.002 | 1.842 | 0.091 | 1.435 |
| bFGF | 0.457 | 0.879 | 0.646 | 0.892 | 0.530 | 0.840 |
| EGF | 0.420 | 0.868 | 0.429 | 0.828 | 0.674 | 0.884 |
| IGF2 | 0.846 | 1.026 | 0.788 | 0.950 | 0.544 | 1.126 |

FIG. 4G: Baseline plasma biomarkers as prognostic factors for HCC

| | | | | | | p-value, | p-value, |
|---|---|---|---|---|---|---|---|
| | | | p-value, | | p-value, | multivariate | multivariate |
| | | | multivariate analysis | | multivariate | analysis, placebo | analysis, soraf pts |
| | | | placebo pts only | | analysis, all pts | pts only | only |
| Biomarker | | | OS | Independent TTP | OS | OS | OS |
| Ang-2 | Baseline | Binned | <0.0001 | 0.016 | 0.004 | 0.021 | 0.021 |
| bFGF | Baseline | Binned | 0.606 | 0.600 | 0.189 | 0.244 | 0.423 |
| EGF | Baseline | Binned | 0.402 | 0.489 | 0.308 | 0.206 | 0.816 |
| IGF-2 | Baseline | Binned | 0.002 | 0.722 | 0.280 | 0.617 | 0.372 |

In summary, the inventors have identified that baseline plasma Ang2 and IGF-2 are prognostic factors for OS in HCC, wherein,

(a) patients with high baseline Ang2 have shorter OS than pts with low Ang2;

(b) patients with high baseline IGF-2 have longer OS than pts with low IGF-2; and

(c) Ang2 remains independently prognostic in multivariate models

Baseline bFGF and EGF are not prognostic for HCC.

Prognostication of Outcome of Cancer Therapy

The present invention also relates to prognostication of the outcome of a patient suffering from HCC, wherein said patient is receiving or scheduled to receive therapeutic treatment (for example, sorafenib), comprising detecting one or more biomarkers in a test sample of said patient and comparing said levels of said biomarkers to a reference standard (for example, median levels of said biomarkers in a population), wherein differential expression of said biomarker in said test sample compared to said reference standard is indicative of said outcome.

In such embodiments, a “good outcome” can be understood to mean improved overall survival and/or prolonged time to progression, whereas a “poor outcome” can be equated with reduced overall survival and/or shorter time to progression.

Other relevant clinical outcomes include, but are not limited to, progression free survival, time to death, disease free survival, time to symptomatic progression, recurrence free survival, time to recurrence, disease state (i.e., progressive, stable, etc.) and response type (partial, complete, etc.).

In a related embodiment, the likelihood that an HCC patient will benefit from sorafenib treatment can be prognosticated by detecting the levels of one or more biomarkers in test sample of said patient and comparing said levels of said biomarkers to a reference standard (for example, median levels of said biomarkers in a population), wherein differential expression of said biomarker in said test sample compared to said reference standard is indicative of said benefit.

“Benefit,” as used herein, is evaluated based on overall survival (OS; in days) and/or time to progression (TTP; in days). Increased overall survival and/or delayed time to progression indicates that the patient is benefitting/likely to benefit from said sorafenib treatment.

One aspect of the aforementioned embodiment is directed to a method for predicting the outcome of sorafenib treatment of a patient suffering from HCC, comprising detecting, in a test sample of said patient, the expression levels of at least one biomarker which is soluble c-Kit (s-c-Kit), hepatocyte growth factor (HGF), phosphorylated ERK (pERK), or angiopoieitin 2 (Ang2), basic fibroblast growth factor (bFGF), insulin-like growth factor (IGF-2) and comparing said levels to a reference standard, wherein differential expression of said biomarker in said test sample compared to said reference standard is indicative of said outcome.

The clinically relevant states described hereinbefore are understood by one skilled in the art. For example, time to progression (TTP) indicates how long it takes a patient's tumor (or multiple tumors) to grow by a pre-defined amount when measuring in a very specific manner (using the RECIST criteria). They are not “progressing to metastasis” or some other state—their tumor(s) is (are) progressing radiologically—i.e. growing by defined standards.

In the aforementioned discussion, biomarker levels were correlated with overall survival (OS) and time to progression (TTP). However, there are many other defined ways of measuring clinical outcomes. Examples include, but are not limited to, for example:

PFS—Progression free survival (related to TTP but not identical)

TTD—Time to death (related to OS but not identical)

DFS—Disease free survival

TTSP—Time to symptomatic progression (where “progression” is not based on tumor size, but based on other clinical symptoms)

RFS—Recurrence free survival

TTR—Time to recurrence (related to RFS, but not identical)

PD—Progressive disease (based on tumor size)

SD—Stable disease (no change in tumor size, or at least very small changes that don't exceed a defined threshold)

PR—Partial response—Indicates that at a given visit, the patient's tumor(s) has(have) shrunk by a predefined amount

CR—Complete response—Indicates complete disappearance of tumor(s).

Although the aforementioned method relates to the detection of any biomarker, for example, tumor biomarker (e.g., phosphorylated ERK) or plasma biomarker, the detection of plasma biomarkers is preferred. Particularly preferred are plasma biomarkers such as s-c-Kit and HGF.

The inventors of the present invention have identified that patients with high plasma c-KIT levels (i.e., >11.3 ng/ml) are more likely to benefit from sorafenib treatment than patients with low plasma c-KIT levels. It was also determined that patients with low plasma HGF levels (i.e., <3.28 ng/mL) are more likely to benefit from sorafenib treatment than those with high plasma HGF levels. On the basis of these experiments, a measured baseline value of 11.3 ng/ml plasma c-Kit or a measured baseline value of 3.28 ng/ml plasma HGF can be reasonably employed as reference standard(s).

The inventors have further identified a significant interaction between Ang2 levels and effects of sorafenib treatment (with respect to overall survival), when Ang2 was monitored as a continuous variable (p for interaction=0.015). It was herein identified that patients with low baseline Ang2 may benefit from sorafenib more than patients with high Ang2. Additionally, patients with high baseline bFGF levels (i.e., >the median value of 7.4 pg/mL) benefit more from sorafenib than those with low bFGF (p for interaction=0.078). Lastly, patients with low baseline IGF-2 levels (i.e., <the median value of 797.7 ng/mL) benefit more from sorafenib treatment than those with high IGF-2 (p for interaction=0.13). On the basis of these studies, a measured baseline value of 7.4 pg/mL plasma bFGF or a measured baseline value of 797.7 ng/mL plasma IGF-2 can be reasonably employed as reference standard(s).

Therefore in the present invention, there is provided a method for predicting the outcome of an HCC patient who is scheduled to receive sorafenib treatment, comprising detecting, in a plasma sample of said patient, the level of s-c-Kit protein or hepatocyte growth factor (HGF) protein, angiopoietin 2 (Ang2) protein, basic fibroblast growth factor (bFGF) protein, or insulin-like growth factor 2 (IGF-2) protein, and comparing said plasma levels of s-c-Kit, HGF, Ang2, bFGF or IGF-2 to a reference standard, wherein elevated levels of said plasma s-c-Kit, elevated levels of said plasma bFGF and/or attenuated levels of said plasma HGF, attenuated levels of said Ang2, attenuated levels of said IGF-2 in said patient compared to said reference standard is indicative of good outcome of said sorafenib treatment.

Similarly, the present invention provides for a method for predicting that a patient suffering from HCC will benefit from sorafenib treatment, comprising detecting, in a plasma sample of said patient, the level of s-c-Kit protein, hepatocyte growth factor (HGF) protein, Ang2 protein, bFGF or IGF-2 and comparing said levels of said s-c-Kit, said HGF, said Ang2, said bFGF, and/or said IGF-2 to a reference standard, wherein elevated levels of said plasma s-c-Kit or said bFGF, either solely or in combination with attenuated levels of said plasma HGF, said Ang2, or said IGF-2 in said patient compared to said reference standard is indicative that said patient will benefit from said sorafenib treatment.

In such embodiments, it is also possible to detect a combination of plasma biomarkers, for example, s-c-Kit and HGF, s-c-Kit and bFGF, c-KIT and IGF-2; c-Kit and Ang2; HGF and Ang2; HGF and bFGF; HGF and IGF-2, bFGF and IGF-2; etc.

Particularly preferred combinations include, but are not limited to, s-c-Kit and HGF; s-c-Kit and bFGF; s-c-KIT and IGF-2; bFGF and IGF-2; HGF and bFGF; HGF and IGF-2; etc.

Also preferred are combinations comprising, for example, s-c-Kit, HGF and bFGF; s-c-Kit, HGF and IGF-2.

The reference standard could comprise experimentally measured biomarker levels in a population, for example, mean or median plasma levels of said biomarkers in HCC patients. Other reference standards, for example, confidence intervals (for example, 95% confidence interval values) or percentiles (for example, 25^{th} percentile or 75^{th} percentile values) may also be employed. Baseline biomarker levels that were observed in a representative patient sample are, for example, set forth in tables 1A and 1B.

Although a population comprising HCC patients is particularly preferred in the establishment of reference standards, the population may comprise normal (i.e., healthy) subjects. The test sample and/or the reference standard can constitute any biological material, although the use of fluids such, for example, blood, urine, sweat, tears, mucus, bile, vaginal fluid, semen and the like are preferred. Most preferred are plasma biomarkers such as, for example, HGF, s-c-Kit, VEGF, sVEGFR2, sVEGFR3, Ang2, bFGF, IGF-2, and the like.

In one such embodiment, s-c-Kit biomarker levels are measured in a test sample and in a reference standard. By the way of example, a median plasma concentration of 11.3 ng/ml s-c-Kit, as determined in a study of HCC patients, can be used as a baseline value. When compared with this reference standard, a given patient's plasma s-c-Kit levels may be classified as being “high” (i.e., >11.3 ng/ml) or “low” (i.e., <11.3 ng/ml). In other embodiments, HGF levels are measured. In such cases, a 75^{th} percentile plasma concentration of 3.28 ng/ml HGF, as determined in a study of HCC patients, can be used to define “low” versus “high” HGF levels.

Yet in other embodiments, VEGF levels can be measured. In such embodiments, a 75^{th} percentile plasma VEGF levels in a population of HCC patients (101.9 pg/ml) can be used as a reference standard for determination of “low” vs. “high” VEGF levels. A 25^{th} percentile plasma s-VEGFR-3 levels in a population of HCC patients (30.559 ng/ml) can be used as a reference standard for determination of “low” vs. “high” s-VEGFR-3 levels.

Yet in other embodiments, Ang2 levels can be measured. In such embodiments, a median (i.e., 50^{th} percentile plasma Ang2 levels in a population of HCC patients (6.061 ng/ml) can be used as a reference standard for determination of “low” vs. “high” Ang2 levels. A 50^{th} percentile plasma bFGF or plasma IGF-2 levels in a population of HCC patients (7.5 pg/ml and 798 ng/ml, respectively, for bFGF and IGF-2) can be used as a reference standard for determination of “low” vs. “high” biomarker levels.

“Sorafenib,” as used hereinbefore, comprises a compound of formula I below or a pharmaceutically acceptable salt, polymorph, hydrate, metabolite, solvate thereof or a combination thereof.

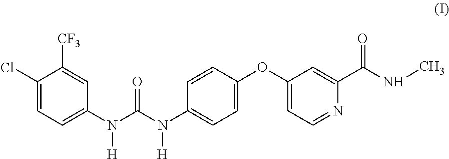

The compounds of formula I and their salts, polymorphs, hydrates and salts are described in U.S. Pat. No. 7,235,576, U.S. Pat. No. 7,351,834, EP 1,140,840B1, WO 03/068746 and WO 04/078746. The disclosures in each of these applications/patents are incorporated by reference in their entirety.

In a preferred embodiment, “sorafenib” comprises a urea compound which is N-[4-chloro-3-(trifluoromethyl)phenyl]-N′-{4-[2-carbamoyl-1-oxo-(4-pyridyloxy)]phenyl}urea or a tosylate salt thereof.

The compounds of formula I can be used solely or in combination with another therapeutic agent, such as, for example, chemotherapeutic agents, immunotherapeutic agents, etc. In the current study sorafenib was used as a single agent. However, there are many ongoing clinical trials using sorafenib in combination with other agents (chemotherapies, immunomodulatory agents, molecularly targeted agents). As such, the biomarkers of the present invention also apply in combination treatments of sorafenib.

Monitoring Cancer Treatment

In some aspects, the present invention provides a method of monitoring the treatment of a patient with cancer, comprising administering sorafenib to the patient and preparing an expression profile from a test sample comprising a plasma sample, serum sample, cell or tissue sample of the patient and comparing the test sample expression profile to an expression profile of a pre-treatment sample from the same individual, or to a reference standard (for example, a plasma or serum sample from a non-HCC population, or cell population comprising normal cells, cancer cells or both) wherein differential expression of at least one biomarker which is s-c-Kit, HGF, Ras p21, s-VEGFR-3, pERK, Ang2, bFGF and/or IGF-2, optionally together with VEGF and/or s-VEGFR-2 in said test sample is indicative of the outcome of the treatment.

In such embodiments, there is provided a method for monitoring an HCC patient undergoing sorafenib treatment, comprising detecting, before and after said sorafenib treatment, the levels of at least one biomarker which is s-c-Kit, HGF, Ras p21, s-VEGFR-3, pERK, Ang2, bFGF, and IGF-2 optionally together with VEGF and/or s-VEGFR-2 in a patient sample, wherein differential expression of at least one said biomarker in said patient sample after sorafenib treatment is indicative of positive outcome of treatment. The duration of sorafenib treatment can be determined by the physician, for example, values at baseline (BL, i.e. pre-treatment), week 12 (cycle 3-day 1 or C3D1), or other time-points may be employed.

The use of plasma biomarkers such as, for example, s-c-Kit, HGF, Ras p21, VEGF, s-VEGFR-2, s-VEGFR-3, IGF-2, and Ang2 are particularly preferred in the aforementioned embodiments.

In a related aspect a combination of the aforementioned plasma biomarkers may be employed. To facilitate the understanding of such combinations, the aforementioned biomarkers are grouped as follows:

Group A comprising HGF, s-c-Kit, s-VEGFR-3, IGF-2 and Ang2;

Group B comprising VEGF, s-VEGFR-2, Ras p21

Preferred combinations include, but are not limited to:

(a) Combinations comprising one biomarker from Group A and one biomarker from Group B

(i) HGF and VEGF;

(ii) s-c-Kit and VEGF;

(iii) s-VEGFR-3 and VEGF;

(iv) HGF and s-VEGFR-2;

(v) s-c-Kit and s-VEGFR-2;

(vi) s-VEGFR-3 and s-VEGFR-2;

(vii) Ang2 and VEGF;

(viii) Ang2 and sVEGFR2;

(ix) Ang2 and Ras p 21;

(x) IGF-2 and VEGF;

(xi) IGF-2 and sVEGFR2;

(xii) IGF-2 and Ras p21; or

(b) Combinations comprising one biomarker from Group A and two biomarkers from Group B

(i) HGF and VEGF plus s-VEGFR-2;

(ii) s-c-Kit and VEGF plus s-VEGFR-2;

(iii) s-VEGFR-3 and VEGF plus s-VEGFR-2;

(iv) Ang2 and VEGF plus sVEGFR2;

(v) Ang2 and sVEGFR2 plus Ras p21;

(vi) Ang2 and Ras p21 plus VEGF;

(vii) IGF-2 VEGF and sVEGFR2;

(viii) IGF-2, sVEGFR2 and Ras p21;

(ix) IGF-2, VEGF and Ras p21; or

(c) Combinations comprising two biomarkers from Group A and one biomarker from Group B

(i) HGF, s-c-Kit and VEGF;

(ii) HGF, s-c-Kit and s-VEGFR-2;

(iii) HGF, s-VEGFR-3 and VEGF;

(iv) HGF, s-VEGFR-3 and s-VEGFR-2;

(v) s-c-Kit, s-VEGFR-3 and VEGF;

(vi) s-c-Kit, s-VEGFR-3 and s-VEGFR-2;

(vii) HGF, Ang2 and VEGF;

(viii) HGF, Ang2 and s-VEGFR-2;

(ix) s-c-Kit, Ang2 and VEGF;

(x) s-c-Kit, s-VEGFR-3 and s-VEGFR-2;

(xi) s-VEGFR-3, Ang2 and VEGF;

(xii) s-VEGFR-3, Ang2 and s-VEGFR-2;

(xiii) IGF-2, HGF and VEGF;

(xiv) IGF-2, HGF and sVEGFR2;

(xv) IGF-2, HGF and Ras p21;

(xvi) IGF-2, Ang2 and VEGF;

(xvii) IGF-2, Ang2 and sVEGFR2;

(xviii) IGF-2, Ang2 and Ras p21;

(xix) IGF-2, s-c-Kit and VEGF;

(xx) IGF-2, s-c-Kit and sVEGFR2;

(xxi) IGF-2, s-c-Kit and Ras p21; or

(d) Combinations comprising two biomarkers from Group A and two biomarkers from Group B

(i) HGF, s-c-Kit and VEGF plus s-VEGFR-2;

(ii) HGF, s-VEGFR-3 and VEGF plus s-VEGFR-2;

(iii) s-c-Kit, s-VEGFR-3 and VEGF plus s-VEGFR-2;

(iv) HGF, Ang2 and VEGF plus s-VEGFR-2;

(v) s-c-Kit, Ang2 and VEGF plus s-VEGFR-2;

(vi) s-VEGFR-3, Ang2 and VEGF plus s-VEGFR-2;

(vii) IGF-2, HGF and VEGF plus sVEGFR2;

(viii) IGF-2, HGF and sVEGFR2 plus Ras p21;

(ix) IGF-2, HGF and VEGF plus Ras p21;

(x) IGF-2, Ang2 and VEGF plus sVEGFR2;

(xi) IGF-2, Ang2 and sVEGFR2 plus Ras p21;

(xii) IGF-2, Ang2 and VEGF plus Ras p21;

(xiii) IGF-2, s-c-Kit VEGF plus sVEGFR2;

(xiv) IGF-2, s-c-Kit and sVEGFR2 plus Ras p21;

(xv) IGF-2, s-c-Kit and VEGF plus Ras p21; or

(e) Combinations comprising three biomarkers from Group A and one biomarker from Group B

(i) HGF, s-c-Kit, s-VEGFR-3 and VEGF;

(ii) HGF, s-c-Kit, s-VEGFR-3 and s-VEGFR-2;

(iii) HGF, s-c-Kit, Ang2 and VEGF;

(iv) HGF, s-c-Kit, Ang2 and s-VEGFR-2;

(vi) s-c-Kit, s-VEGFR-3, Ang2 and VEGF;

(vi) s-c-Kit, s-VEGFR-3, Ang2 and s-VEGFR-2;

(vii) HGF, s-VEGFR-3, Ang2 and VEGF;

(viii) HGF, s-VEGFR-3, Ang2 and s-VEGFR-2;

(ix) HGF, s-c-Kit, IGF-2 and VEGF;

(x) HGF, s-c-Kit, IGF-2 and s-VEGFR-2;

(xi) HGF, IGF-2, Ang2 and VEGF;

(xii) HGF, IGF-2, Ang2 and s-VEGFR-2; or

(f) Combination comprising three biomarkers from Group A and two biomarkers from Group B

(i) HGF, s-c-Kit, s-VEGFR-3 and VEGF plus s-VEGFR-2;

(ii) HGF, s-c-Kit, Ang2 and VEGF plus s-VEGFR-2;

(iii) HGF, Ang2, s-VEGFR-3 and VEGF plus s-VEGFR-2;

(iv) s-c-Kit, s-VEGFR-3, Ang2 and VEGF plus s-VEGFR-2;

(v) HGF, s-c-Kit, IGF-2 and VEGF plus s-VEGFR-2;

(vi) HGF, s-c-Kit, IGF-2 and VEGF plus s-VEGFR-2;

(vii) HGF, IGF-2, Ang2 and VEGF plus s-VEGFR-2;

(viii) HGF, IGF-2, Ang2 and VEGF plus s-VEGFR-2; or

(g) Combination comprising four biomarkers from Group A and one biomarker from Group B

(i) HGF, s-c-Kit, s-VEGFR-3, Ang2 and VEGF;

(ii) HGF, s-c-Kit, s-VEGFR-3, Ang2 and s-VEGFR-2;

(iii) HGF, s-c-Kit, IGF-2, Ang2 and VEGF;

(iv) HGF, s-c-Kit, IGF-2, Ang2 and s-VEGFR-2; or

(h) Combination comprising four biomarkers from Group A and two biomarkers from Group B

(i) HGF, s-c-Kit, s-VEGFR-3, Ang2 and VEGF plus s-VEGFR-2;

(ii) HGF, s-c-Kit, IGF-2, Ang2 and VEGF plus s-VEGFR-2; or

(i) Combinations comprising all of the aforementioned biomarkers;

The above baseline plasma biomarkers were obtained for the patient class investigated. It is foreseen that baseline values may vary for a patient class and the invention is not limited to the use of these baseline values.

The inventors of the present application have identified that levels of plasma c-KIT, HGF, Ras p21, s-VEGFR-2, and s-VEGFR-3 biomarkers are attenuated in sorafenib-treated patients compared to controls (i.e., baseline levels) while plasma VEGF levels are elevated. Results are summarized in the tables (Tables 5A and 6).

The inventors of the present application have also identified that levels of plasma Ang2, biomarkers are attenuated in sorafenib-treated patients compared to controls (i.e., baseline levels), while plasma Ang2 levels increase in placebo patients compared to controls (i.e., baseline levels. Results are summarized in the tables (Tables 5B).

TABLE 5A
Cycle 3 day 1 (C3D1) changes in biomarker levels (p ≦ 0.05 indicates significance)

| | | | | | | Soraf vs |
|---|---|---|---|---|---|---|
| | | Placebo | | Sorafenib | | Pla |
| | | | Δ from BL | | Δ from BL | p-value for |
| Biomarker | | Mean (d) | (e) | Mean (d) | (e) | C3D1 |
| c-KIT | Baseline | 11.9 | NA | 12.2 | NA | NA |
| (ng/mL) | C3D1 | 11.5 | −0.8 | 8.6 | −4.5 | <0.0001 |
| HGF | Baseline | 2877.3 | NA | 2630.0 | NA | NA |
| (pg/mL) | C3D1 | 3197.8 | 371.0 | 2220.3 | −285.0 | <0.0001 |
| Ras p21 | Baseline | 1677.3 | NA | 2115.7 | NA | NA |
| (pg/mL) | C3D1 | 1336.1 | 55.8 | 1144.7 | −259.8 | 0.046 |
| VEGF | Baseline | 88.5 | NA | 100.7 | NA | NA |
| (pg/mL) | C3D1 | 102.1 | 19.9 | 170.1 | 66.8 | 0.010 |
| VEGFR-2 | Baseline | 8974.3 | NA | 8773.4 | NA | NA |
| (pg/mL) | C3D1 | 9087.6 | 154.7 | 6395.6 | −2295.8 | <0.0001 |
| VEGFR-3 | Baseline | 42000.8 | NA | 44754.1 | NA | NA |
| (pg/mL) | C3D1 | 45950.2 | 5038.6 | 37903.8 | -6826.3 | <0.0001 |

TABLE 5B
Cycle 3 day 1 (C3D1) changes in biomarker levels (p ≦ 0.05 indicates significance)

| | | | | | | Soraf vs |
|---|---|---|---|---|---|---|
| | | Placebo | | Sorafenib | | Pla |
| | | | Δ from BL | | Δ from BL | p-value for |
| Biomarker | | Mean (d) | (e) | Mean (d) | (e) | C3D1 |
| Ang2 | Baseline | 7718.1 | NA | 7510.0 | NA | NA |
| (pg/mL) | C3D1 | 8847.5 | 1916.9 | 6456.4 | −293.0 | <0.0001 |
| bFGF | Baseline | 14.9 | NA | 14.1 | NA | NA |
| (pg/mL) | C3D1 | 16.1 | 2.7 | 16.1 | 2.5 | 0.196 |
| EGF | Baseline | 59.3 | NA | 61.8 | NA | NA |
| (pg/mL) | C3D1 | 63.9 | 1.1 | 53.4 | −8.9 | 0.240 |
| IGF-2 | Baseline | 888.2 | NA | 938.8 | NA | NA |
| (ng/mL) | C3D1 | 815.2 | −94.3 | 847.9 | −129.4 | 0.145 |

(d) Mean was calculated for all subjects with biomarker data available at this timepoint
(e) Absolute change from BL was calculated individually for each subject and then changes for all subjects were averaged

It was further identified that plasma EGF mean level decreases during sorafenib treatment (p=0.025*), while plasma IGF-2 mean level decreases during sorafenib treatment (p<0.0001*) and during placebo treatment (p<0.0001*).

TABLE 6A
Change in plasma biomarker levels in response to
sorafenib treatment

| | Direction of | |
|---|---|---|
| | change in | Mean change |
| | response to | in sorafenib |
| Biomarker | sorafenib | arm |
| c-KIT | [symbol] | −33.9% |
| HGF | [symbol] | −7.4% |
| Ras p21 | [symbol] * | −259.8 pg/mL* |
| VEGF | [symbol] | +195.7% |
| s-VEGFR-2 | [symbol] | −25.7% |
| s-VEGFR-3 | [symbol] | −14.1% |

TABLE 6B
Change in plasma biomarker levels in response to
sorafenib treatment

| | Direction of | Mean | Direction of | Mean |
|---|---|---|---|---|
| | change in | change | change in | change |
| Biomarker | sorafenib | in sorafenib | placebo | in placebo |
| Ang2 | None | | [symbol] | +35.6% |
| bFGF | None | | None | |
| EGF | Mixed | | None | |
| IGF-2 | [symbol] | −11.3% | [symbol] | −8.1% |

Thus in the present invention, there is provided a method for monitoring the response of an HCC patient towards sorafenib treatment comprising

detecting a baseline level of at least one biomarker which is s-c-Kit, HGF, Ras p21, VEGF, s-VEGFR-2, s-VEGFR-3, Ang2, or IGF-2 in a test sample of said patient before sorafenib treatment,

detecting the level of said at least one biomarker in said test sample of said patient after sorafenib treatment, and

comparing said after sorafenib treatment biomarker level to said before sorafenib treatment baseline level,

wherein an attenuation in the levels of at least one of s-c-Kit, HGF, Ras p21, s-VEGFR-2, s-VEGFR-3 or Ang2 and/or an elevation in the levels of VEGF or an increase or only modest decrease in IGF-2 in said test sample after sorafenib treatment is indicative that said patient is responsive to said sorafenib treatment.

Novel Biomarkers which Indicate Drug Efficacy

The present inventors have identified novel plasma biomarkers whose changes during the course of a therapeutic regimen (for example, sorafenib treatment) are correlated with the outcome of the therapeutic regimen. Preferably, the biomarker is a plasma biomarker such as, for example, s-c-Kit, HGF, Ras p21, VEGF, s-VEGFR-2, s-VEGFR-3, Ang2, and IGF-2 whose expression profile is changed during the course of sorafenib treatment.

“Course of treatment” as used herein, may comprise two, or more, time points. For example, the first time point may comprise measurement of said biomarker levels before sorafenib treatment and the later time point comprises measurement at week 12 (cycle 3 day 1 or C3D1) of sorafenib treatment. A third time point, which falls in between these two time points, may be additionally used. Additional time-points may also be used.

More specifically, the inventors have identified that a reduction in plasma HGF levels of at least 294 pg/mL (i.e., median plasma HGF levels) at cycle 3 day 1 (C3D1) of sorafenib treatment is associated with significantly longer time to progression.

As a non-limiting example, which is to be used for illustrative purposes only, the method may comprise measuring plasma HGF levels before sorafenib treatment and at cycle 3 day 1 (C3D1); determining the change in said plasma HGF levels; and comparing said change with a reference value of 294 pg/mL plasma HGF, wherein a change in plasma HGF levels of >294 pg/mL at C3D1 indicates significantly longer time to progression.

The inventors have further identified that plasma Ang2 levels remain unchanged in sorafenib-treated patients (i.e., median plasma Ang2 levels stays the same) at cycle 3 day 1 (C3D1) while in placebo patients, plasma levels of Ang2 increases significantly at C3D1. In this patient cohort, it was found that sorafenib patients with Ang2 decrease have longer overall survival than patients with Ang2 increase (p<0.001). In this patient cohort, it was found that sorafenib patients with Ang2 decrease have significantly longer time to progression than patients with Ang2 increase (p=0.005). In both studies measuring the association of plasma Ang2 levels with OS and TTP in sorafenib-treated patients, similar results were observed when bifurcate change in median Ang2 levels (instead of 0%) was employed. The median change in Ang2 in all groups was calculated to be 5.1%.

Ang2 levels were also prognostic in placebo patients, wherein placebo patients with Ang2 decrease at C3D1 have longer OS than patients with Ang2 increase (p<0.0001). Moreover, placebo patients with Ang2 decrease at C3D1 also have longer TTP than patients with Ang2 increase.

The inventors have further identified that median IGF-2 levels were attenuated at C3D1 in HCC patients. The median change in IGF-2 levels in all patients was 94.3 ng/mL. Interestingly, sorafenib-treated patients with change in plasma IGF-2 levels that are greater than median change (i.e., greater than 94.3 ng/mL) have longer OS than sorafenib-treated patients with IGF-2 change that was less than the median (i.e., less than 94.3 ng/mL) (p=0.011). Moreover, sorafenib-treated patients with change in plasma IGF-2 levels that are greater than median change (i.e., greater than 94.3 ng/mL) have longer TTP than sorafenib-treated patients with IGF-2 change that was less than the median (i.e., less than 94.3 ng/mL) (p=0.008).

Also interestingly, placebo patients with change in plasma IGF-2 levels that are greater than median change (i.e., greater than 94.3 ng/mL) have longer OS than sorafenib-treated patients with IGF-2 change that was less than the median (i.e., less than 94.3 ng/mL) (p=0.002). Moreover, placebo patients with change in plasma IGF-2 levels that are greater than median change (i.e., greater than 94.3 ng/mL) have longer TTP than sorafenib-treated patients with IGF-2 change that was less than the median (i.e., less than 94.3 ng/mL). Consistent results were obtained when bifurcate change in IGF-2 was studied (instead of median changes).

The inventors have further identified that median IGF-2 levels were attenuated at C3D1 in HCC patients. The median change in IGF-2 levels in all patients was 11.2%. Sorafenib-treated patients with change in plasma IGF-2 levels that are greater than median change (i.e., greater than 11.2%) have longer OS than sorafenib-treated patients with IGF-2 change that was less than the median (i.e., less than 11.2%) (p=0.063).

An analysis of C3D1 change in biomarker levels for the six plasma biomarkers and the association thereof with OS and TTP is presented in the tables below (Tables 7A and 7B).

Therefore in one embodiment, the present invention provides a method for evaluating the efficacy of sorafenib treatment in a patient suffering from HCC, comprising

detecting the levels of plasma HGF, Ang2, or IGF-2 in said patient at one time point;

detecting the levels of plasma HGF, Ang2, or IGF-2 in said patient at a later time point; and

comparing said plasma HGF, Ang2, or IGF-2 levels in said patient at the two time points;

wherein a reduction in said plasma HGF, Ang2 or IGF-2 levels in said patients at said later time point is indicative of said efficacy of sorafenib treatment.

In a most preferred embodiment, the present invention provides a method for prognosticating overall survival in an HCC patient receiving sorafenib treatment, comprising

detecting the levels of plasma HGF, Ang2, or IGF-2 in said patient at one time point;

detecting the levels of plasma HGF, Ang2, or IGF-2 in said patient at a later time point; and

comparing said plasma HGF, Ang2, or IGF-2 levels in said patient at the two time points;

wherein a reduction of plasma HGF levels, reduction of plasma Ang2 or reduction of IGF-2 levels at said later time point is indicative of increased time to progression of said HCC.

In a related embodiment, the present invention provides a method for prognosticating the time to progression in an HCC patient receiving sorafenib treatment, comprising

detecting the levels of plasma HGF, Ang2, or IGF-2 in said patient at one time point;

detecting the levels of plasma HGF, Ang2, or IGF-2 in said patient at a later time point; and

comparing said plasma HGF, Ang2, or IGF-2 levels in said patient at the two time points;

wherein a reduction of plasma HGF levels, reduction of plasma Ang2 or reduction of IGF-2 levels at said later time point is indicative of increased time to progression of said HCC.

As described hereinbefore, any combination of HGF, Ang2 and/or IGF-2 may also be employed. Preferred combinations include, but are not limited to, HGF and Ang2, HGF and IGF-2, Ang2 and IGF-2, and HGF, Ang2 and IGF-2. Purely to facilitate understanding, the invention provides a method for prognosticating the outcome in an HCC patient receiving sorafenib treatment, comprising

detecting the levels of a combination of biomarkers which is plasma HGF, plasma Ang2, or plasma IGF-2 in said patient at one time point;

detecting the levels of said combination of said biomarkers in said patient at a later time point; and

comparing said combination of biomarker levels in said patient at the two time points;

wherein a reduction in the levels of said combination of biomarkers at said later time point is indicative of increased time to progression of said HCC.

Additional parameters such as, for example, distant metastasis, presence of secondary tumors, level of differentiation, response to chemotherapy (for example, sorafenib treatment), etc. may be used in characterization of the HCC tumors.

TABLE 7A
Sorafenib-associated C3D1 change in plasma biomarker level
(as compared to baseline) and outcome.
(p ≦ 0.1 indicates significance)

| | | p-value for high BioM change vs low | | |
|---|---|---|---|---|
| | | change, soraf pts only | | |
| | | | Independent | Investigator |
| Biomarker | | OS | TTP | TTP |
| c-KIT | Absolute Δ | 0.663 | 0.289 | 0.922 |
| | BL-C3D1 | | | |
| | % Δ | 0.930 | 0.540 | 0.683 |
| | BL-C3D1 | | | |
| HGF | Absolute Δ | 0.960 | 0.029 | 0.052 |
| | BL-C3D1 | | | |
| | % Δ | 0.147 | 0.083 | 0.016 |
| | BL-C3D1 | | | |
| Ras p21 | Absolute Δ | 0.191 | 0.168 | 0.580 |
| | BL-C3D1 | | | |
| | % Δ | 0.123 | 0.092 | 0.958 |
| | BL-C3D1 | | | |
| VEGF | Absolute Δ | 0.569 | 0.597 | 0.955 |
| | BL-C3D1 | | | |
| | % Δ | 0.914 | 0.446 | 0.973 |
| | BL-C3D1 | | | |
| VEGFR-2 | Absolute Δ | 0.480 | 0.697 | 0.311 |
| | BL-C3D1 | | | |
| | % Δ | 0.177 | 0.835 | 0.992 |
| | BL-C3D1 | | | |
| VEGFR-3 | Absolute Δ | 0.183 | 0.803 | 0.199 |
| | BL-C3D1 | | | |
| | % Δ | 0.141 | 0.822 | 0.271 |
| | BL-C3D1 | | | |

TABLE 7B
Sorafenib-associated C3D1 change in plasma biomarker level
(as compared to baseline) and outcome.
(p ≦ 0.1 indicates significance)

| | | p-value for high BioM change vs low changs | | | |
|---|---|---|---|---|---|
| | | Sorafenib pts only | | Placebo pts only | |
| | | | Independent | | Independent |
| Biomarker | | OS | TTP | OS | TTP |
| Ang2 | Absolute | 0.001 | 0.005 | <0.0001 | 0.002 |
| | Δ BL-C3D1, | | | | |
| | 0 split | | | | |
| | Absolute | <0.0001 | 0.001 | <0.0001 | <0.0001 |
| | Δ BL-C3D1, | | | | |
| | median split | | | | |
| | % Δ BL-C3D1, | 0.001 | 0.005 | <0.0001 | 0.002 |
| | median split | | | | |
| bFGF | Absolute | 0.198 | 0.875 | 0.407 | 0.320 |
| | Δ BL-C3D1, | | | | |
| | 0 split | | | | |
| | Absolute | 0.155 | 0.729 | 0.326 | 0.191 |
| | Δ BL-C3D1, | | | | |
| | median split | | | | |
| | % Δ BL-C3D1, | 0.021 | 0.414 | 0.890 | 0.140 |
| | median split | | | | |
| EGF | Absolute | 0.174 | 0.875 | 0.654 | 0.351 |
| | Δ BL-C3D1, | | | | |
| | 0 split | | | | |
| | Absolute | 0.796 | 0.648 | 0.391 | 0.289 |
| | Δ BL-C3D1, | | | | |
| | median split | | | | |
| | % Δ BL-C3D1, | 0.796 | 0.648 | 0.391 | 0.289 |
| | median split | | | | |
| IGF-2 | Absolute | 0.102 | 0.742 | <0.001 | 0.046 |
| | Δ BL-C3D1, | | | | |
| | 0 split | | | | |
| | Absolute | 0.011 | 0.008 | 0.002 | 0.025 |
| | Δ BL-C3D1, | | | | |
| | median split | | | | |
| | % Δ BL-C3D1, | 0.063 | 0.675 | <0.0001 | 0.030 |
| | median split | | | | |

The invention also relates to a mode of classification of a cancer patient according to a combination of the aforementioned parameters (for example, improved survival group with increased time to progression, reduced survival group with reduced time to progression, etc). The utility of such parameters in the calculation of international prognostication index (IPI) is known in the art.

Tumor Biomarkers

The invention further relates to novel tumor biomarkers whose expression and/or activity is modulated in response to sorafenib treatment.

In one embodiment, there is provided a method for prognosticating the outcome of a patient suffering from HCC, comprising

detecting, in a test tumor sample of said patient, the levels of phospho-ERK (pERK); and

comparing said levels of pERK with a reference standard;

wherein differential expression of said pERK in said tumor sample compared to a reference standard is indicative of the outcome of said HCC.

The inventors of the present application have identified that elevated levels of pERK in the tumor is significantly correlated with a longer TTP upon sorafenib treatment. In such studies, the baseline tumor pERK levels in HCC patients are first determined using art known techniques (for example, immunostaining), based on which a particular tumor sample is characterized as having “high” or “low” pERK levels.

In the present invention, pERK levels are scored by immunohistochemistry (IHC) analysis of a tumor sample. There are currently two main ways that pathologists score protein levels in IHC: intensity and % area stained (they can also use permutations of these, such as area stained above a certain intensity level, or multiplying intensity level by total area stained, or assessing % of cells with nuclei stained positive, etc).

As a representative example, in the present invention, a staining intensity (how dark the stain is) based scoring procedure, comprising a value of 0−4+ was used. The scale of the scoring intensity is thus 0, 1+, 2+, 3+, 4+, where 4+ is most intense and 0 is no staining.

In the present invention, cancer patients who had high pERK (defined as having a maximum intensity score of 3+ or 4+) benefited more from sorafenib treatment than those with low pERK (defined as max intensity score of 0, 1+ or 2+). The same results were observed when % area stained positive was used in the analysis. As such, patients with positive staining over >5% of the tumor area benefited more from sorafenib than those with staining over 0-5% of the tumor area. Based on these studies, one skilled in the art can assign additional cut-off values, (i.e., 0-10% versus >10% staining) for correlating pERK levels with outcome.

In a preferred embodiment, there is provided a method for prognosticating the time to progression of a patient suffering from HCC, comprising

detecting, in a test tumor sample of said patient, the levels of phospho-ERK (pERK); and

comparing said levels of pERK with a reference standard comprising measured pERK levels in a population of said HCC patients;

wherein elevated expression of said pERK in said tumor sample compared to said reference standard is indicative of the outcome of said HCC.

Prognostication of Tumors

The present invention also relates to prognostication of the outcome of a patient suffering from HCC, wherein said patient is receiving or scheduled to receive chemotherapeutic treatment (for example, sorafenib), comprising detecting one or more tumor biomarkers in a test tumor sample of said patient. In such embodiment, the effect of sorafenib treatment on overall survival (OS) or time to progression (TTP) be prognosticated by detecting the levels of phospho-ERK (pERK) in said patient and comparing said levels to a reference standard (for example, median pERK in tumor samples, as determined by antibody staining), wherein elevated levels of said pERK in said test tumor sample compared to said reference standard is indicative of improved overall survival and/or time to progression.

Methods for Screening for a Bioactive Agent

The present invention includes methods of screening for an agent capable of modulating the outcome of HCC in a subject, comprising contacting a tumor cell to the agent; and detecting the expression level of at least one biomarker which is s-c-Kit, HGF, Ras p21, VEGF, s-VEGFR-2, s-VEGFR-3, pERK, Ang2, bFGF or IGF-2 wherein differential expression of said biomarker in said tumor cell compared to a reference standard is indicative of an agent which is capable of modulating the outcome of said HCC.

The present inventors have identified that sorafenib treatment increases time to progression and/or overall survival of HCC compared to placebo treated subjects. In these patients, an attenuation of s-c-Kit, HGF, Ras p21, s-VEGFR-2, and/or s-VEGFR-3 biomarker levels and/or elevation of VEGF and/or decrease in pERK biomarker levels was concomitantly observed.

As such, the instant invention provides for a method of screening for an agent capable of influencing the outcome of patients with HCC (for example, increasing time to progression and/or improving survival), comprising

contacting a tumor cell to the agent; and

detecting the expression level of at least one biomarker which is s-c-Kit, HGF, Ras p21, VEGF, s-VEGFR-2, s-VEGFR-3, or pERK before and after contacting with said agent;

wherein attenuation in the levels of s-c-Kit, HGF, Ras p21, s-VEGFR-2, or s-VEGFR-3 and/or elevation in the levels of VEGF or decrease in pERK after contacting with said agent indicates that said agent is capable of influencing the outcome of said HCC.

Antibodies and Arrays Directed Thereto

In a related embodiment, the invention is drawn to antibody molecules which specifically bind to vascular endothelial growth factor (VEGF), soluble VEGF receptor 2 (s-VEGFR-2), soluble VEGF receptor 3 (VEGFR-3), soluble s-c-Kit (s-c-Kit), hepatocyte growth factor (HGF), Ras p 21, phosphorylated ERK (pERK), angiopoietin 2 (Ang2), basic fibroblast growth factor (bFGF), insulin-like growth factor (IGF-2). A control antibody which specifically binds to epidermal growth factor (EGF) may also be employed.

The present invention also relates to antibody micro-arrays comprising a plurality of such antibody molecules. Preferably, such antibody-microarrays comprise antibody molecules which specifically bind to the aforementioned proteins in their native (non-denatured) form. Antibody molecules containing detectable labels, including methods for labeling such are known in the art.

Antibody arrays of the present invention may contain a plurality of antibody molecules which specifically bind to at least 2, 3, 4, 5, 6, 7, 8, 9 or more of the aforementioned proteins. Preferred methods may detect all or nearly all of the protein biomarkers. Any combination of antibody-based detection may be employed, for example, detecting a set of proteins that are elevated and/or a set of proteins that are attenuated in response to treatment with sorafenib.

To facilitate the understanding of such arrays, the aforementioned biomarkers, which comprise antigens that bind to the antibodies of the present invention, are grouped as follows:

Group A comprising HGF, s-c-Kit, s-VEGFR-3, IGF-2 and Ang2;

Group B comprising VEGF, s-VEGFR-2, Ras p21

Preferred arrays comprise, but are not limited to:

(a) Antibody molecule(s) which bind to one biomarker from Group A and one biomarker from Group B, wherein the biomarkers are:

(i) HGF and VEGF;

(ii) s-c-Kit and VEGF;

(iii) s-VEGFR-3 and VEGF;

(iv) HGF and s-VEGFR-2;

(v) s-c-Kit and s-VEGFR-2;

(vi) s-VEGFR-3 and s-VEGFR-2;

(vii) Ang2 and VEGF;

(viii) Ang2 and sVEGFR2;

(ix) Ang2 and Ras p 21;

(x) IGF-2 and VEGF;

(xi) IGF-2 and sVEGFR2;

(xii) IGF-2 and Ras p21; or

(b) Antibody molecule(s) which bind to one biomarker from Group A and two biomarkers from Group B, wherein the biomarkers are:

(i) HGF and VEGF plus s-VEGFR-2;

(ii) s-c-Kit and VEGF plus s-VEGFR-2;

(iii) s-VEGFR-3 and VEGF plus s-VEGFR-2;

(iv) Ang2 and VEGF plus sVEGFR2;

(v) Ang2 and sVEGFR2 plus Ras p21;

(vi) Ang2 and Ras p21 plus VEGF;

(vii) IGF-2 VEGF and sVEGFR2;

(viii) IGF-2, sVEGFR2 and Ras p21;

(ix) IGF-2, VEGF and Ras p21; or

(c) Antibody molecule(s) which bind to two biomarkers from Group A and one biomarker from Group B, wherein the biomarkers are:

(i) HGF, s-c-Kit and VEGF;

(ii) HGF, s-c-Kit and s-VEGFR-2;

(iii) HGF, s-VEGFR-3 and VEGF;

(iv) HGF, s-VEGFR-3 and s-VEGFR-2;

(v) s-c-Kit, s-VEGFR-3 and VEGF;

(vi) s-c-Kit, s-VEGFR-3 and s-VEGFR-2;

(vii) HGF, Ang2 and VEGF;

(viii) HGF, Ang2 and s-VEGFR-2;

(ix) s-c-Kit, Ang2 and VEGF;

(x) s-c-Kit, s-VEGFR-3 and s-VEGFR-2;

(xi) s-VEGFR-3, Ang2 and VEGF;

(xii) s-VEGFR-3, Ang2 and s-VEGFR-2;

(xiii) IGF-2, HGF and VEGF;

(xiv) IGF-2, HGF and sVEGFR2;

(xv) IGF-2, HGF and Ras p21;

(xvi) IGF-2, Ang2 and VEGF;

(xvii) IGF-2, Ang2 and sVEGFR2;

(xviii) IGF-2, Ang2 and Ras p21;

(xix) IGF-2, s-c-Kit and VEGF;

(xx) IGF-2, s-c-Kit and sVEGFR2;

(xxi) IGF-2, s-c-Kit and Ras p21; or

(d) Antibody molecule(s) which bind to two biomarkers from Group A and two biomarkers from Group B, wherein the biomarkers are:

(i) HGF, s-c-Kit and VEGF plus s-VEGFR-2;

(ii) HGF, s-VEGFR-3 and VEGF plus s-VEGFR-2;

(iii) s-c-Kit, s-VEGFR-3 and VEGF plus s-VEGFR-2;

(iv) HGF, Ang2 and VEGF plus s-VEGFR-2;

(v) s-c-Kit, Ang2 and VEGF plus s-VEGFR-2;

(vi) s-VEGFR-3, Ang2 and VEGF plus s-VEGFR-2;

(vii) IGF-2, HGF and VEGF plus sVEGFR2;

(viii) IGF-2, HGF and sVEGFR2 plus Ras p21;

(ix) IGF-2, HGF and VEGF plus Ras p21;

(x) IGF-2, Ang2 and VEGF plus sVEGFR2;

(xi) IGF-2, Ang2 and sVEGFR2 plus Ras p21;

(xii) IGF-2, Ang2 and VEGF plus Ras p21;

(xiii) IGF-2, s-c-Kit VEGF plus sVEGFR2;

(xiv) IGF-2, s-c-Kit and sVEGFR2 plus Ras p21;

(xv) IGF-2, s-c-Kit and VEGF plus Ras p21; or

(e) Antibody molecule(s) which bind to three biomarkers from Group A and one biomarker from Group B, wherein the biomarkers are:

(i) HGF, s-c-Kit, s-VEGFR-3 and VEGF;

(ii) HGF, s-c-Kit, s-VEGFR-3 and s-VEGFR-2;

(iii) HGF, s-c-Kit, Ang2 and VEGF;

(iv) HGF, s-c-Kit, Ang2 and s-VEGFR-2;

(vi) s-c-Kit, s-VEGFR-3, Ang2 and VEGF;

(vi) s-c-Kit, s-VEGFR-3, Ang2 and s-VEGFR-2;

(vii) HGF, s-VEGFR-3, Ang2 and VEGF;

(viii) HGF, s-VEGFR-3, Ang2 and s-VEGFR-2;

(ix) HGF, s-c-Kit, IGF-2 and VEGF;

(x) HGF, s-c-Kit, IGF-2 and s-VEGFR-2;

(xi) HGF, IGF-2, Ang2 and VEGF;

(xii) HGF, IGF-2, Ang2 and s-VEGFR-2; or

(f) Antibody molecule(s) which bind to three biomarkers from Group A and two biomarkers from Group B, wherein the biomarkers are:

(i) HGF, s-c-Kit, s-VEGFR-3 and VEGF plus s-VEGFR-2;

(ii) HGF, s-c-Kit, Ang2 and VEGF plus s-VEGFR-2;

(iii) HGF, Ang2, s-VEGFR-3 and VEGF plus s-VEGFR-2;

(iv) s-c-Kit, s-VEGFR-3, Ang2 and VEGF plus s-VEGFR-2;

(v) HGF, s-c-Kit, IGF-2 and VEGF plus s-VEGFR-2;

(vi) HGF, s-c-Kit, IGF-2 and VEGF plus s-VEGFR-2;

(vii) HGF, IGF-2, Ang2 and VEGF plus s-VEGFR-2;

(viii) HGF, IGF-2, Ang2 and VEGF plus s-VEGFR-2; or

(g) Antibody molecule(s) which bind to four biomarkers from Group A and one biomarker from Group B, wherein the biomarkers are:

(i) HGF, s-c-Kit, s-VEGFR-3, Ang2 and VEGF;

(ii) HGF, s-c-Kit, s-VEGFR-3, Ang2 and s-VEGFR-2;

(iii) HGF, s-c-Kit, IGF-2, Ang2 and VEGF;

(iv) HGF, s-c-Kit, IGF-2, Ang2 and s-VEGFR-2; or

(h) Antibody molecule(s) which bind to four biomarkers from Group A and two biomarkers from Group B, wherein the biomarkers are:

(i) HGF, s-c-Kit, s-VEGFR-3, Ang2 and VEGF plus s-VEGFR-2;

(ii) HGF, s-c-Kit, IGF-2, Ang2 and VEGF plus s-VEGFR-2; or

(i) Combinations comprising all of the aforementioned biomarkers;

Kits, Biochips and Datasets

The invention further comprises kits useful for the practice of one or more of the methods of the invention. In some preferred embodiments, a kit may contain one or more solid supports having attached thereto one or more of the aforementioned antibodies. The solid support may be a high-density antibody array. Kits may further comprise one or more reagents for use with the arrays, one or more signal detection and/or array-processing instruments, one or more protein databases and one or more analysis and database management software packages.

In a preferred embodiment, the instant invention relates to a biochip comprising a plurality of antibodies which specifically bind to the aforementioned polypeptides. Preferred biochips comprise at least 2, at least 3, at least 4, at least 5, at least 6, at least 7, at least 8, at least 9, or all of the proteins from the group consisting of vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin 2 (Ang2), basic fibroblast growth factor (bFGF), insulin-like growth factor (IGF-2). A control antibody which specifically binds to epidermal growth factor (EGF) may also be employed.

The invention includes methods of using the databases, such as methods of using computer systems to present information identifying the expression level in a tissue or cell of at least two of the aforementioned proteins, comprising the step of comparing the expression level of at least one protein in the tumor tissue or cell to the level of expression of the protein in the database. In some preferred embodiments, the method is drawn to the detection of the expression level of one or more of vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin 2 (Ang2), basic fibroblast growth factor (bFGF), insulin-like growth factor (IGF-2). A control antibody which specifically binds to epidermal growth factor (EGF) may also be employed.

The skilled artisan is aware of the fact that many biological functions are accomplished by altering the expression of various proteins and/or activity thereof. For example, fundamental biological processes such as cell cycle, cell differentiation and cell death, are often characterized by the variations in the expression levels of groups of proteins involved in an ingenuity pathway. Examples of such ingenuity pathways, and the relationship of the genes of the forgoing to such pathways, are described below. Changes in the activity of the proteins brought about by post-translational modification events (such as phosphorylation) also are associated with pathogenesis. For example, the lack of sufficient expression of functional tumor suppressors and/or the over-expression of onco-proteins could lead to tumorigenesis or hyperplastic growth of cells (Marshall, (1991) Cell, 64, 313-326; Weinberg, (1991) Science, 254, 1138-1146). Thus, changes in the expression levels of particular proteins (e.g., onco-proteins or tumor suppressors) serve as signposts for the presence and progression of various tumors. The instant invention therefore also relates to a method of ingenuity pathway analysis of a broad spectrum of tumors comprising detecting one or more proteins. For example, in the present invention there is provided a method for the grouping proteins into one or more signature profiles comprising one or more of vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21 or phosphorylated ERK (pERK). Examples of such signature profiles include, but are not limited to, growth receptor ligands (VEGF, HGF, Ang2, bFGF, IGF-2), growth receptors (s-VEGFR2 and s-VEGFR-3), proliferation/survival proteins (Ras p21 and pERK), etc.

Oligonucleotides and Arrays Based Thereon

The invention comprises oligonucleotide arrays which are useful for the practice of one or more of the methods of the invention. Such arrays may contain an oligonucleotide which specifically hybridizes to a gene encoding vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin 2 (Ang2), basic fibroblast growth factor (bFGF), insulin-like growth factor (IGF-2). A control oligonucleotide which specifically hybridizes to a gene encoding epidermal growth factor (EGF) may also be employed.

Preferably, such arrays may comprise a plurality of oligonucleotides which specifically hybridize to at least 2, at least 3, at least 4, at least 5 or at least 6, at least 7, at least 8, at least 9, or more of the aforementioned genes. Preferred methods may detect all or nearly all of the aforementioned genes. Any combination of genes may be employed, for example, a set of genes that are up-regulated and a set of genes that are down-regulated.

The invention also relates to primers and/or probes for measuring the level of expression of vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin 2 (Ang2), basic fibroblast growth factor (bFGF), insulin-like growth factor (IGF-2). in a sample. The primers and/or probes may be designed by using art known techniques based on the structural information (i.e., accession numbers). The genes can be measured by any method common in the art such as PCR, in situ hybridization, sequencing, etc.

Assay Techniques

Reference Standards

In one embodiment the biomarker levels are grouped as percentiles within or based on a set of patient samples, such as all patients with HCC. In such embodiments, a threshold level of expression is established wherein higher or lower levels of expression relative to, for instance, a particular percentile, is used as the basis for predicting outcome. The reference standard could also be defined by biomarker levels in a non-HCC population (for example, healthy subjects, or patients with liver cirrhosis, hepatitis B virus, and/or hepatitis C virus, but without HCC).

Biomarker Detection

In one embodiment the levels of biomarkers are measured using an antibody-based detection strategy [for example, enzyme-linked immuosorbent assay (ELISA), immunoblotting (WB) or immunohistochemistry (IHC)]. However, the aforementioned method is not limited to antibody-based assays. Any method of detection of the expression of the gene and/or polypeptide products thereof can be reliably employed. Such method include, but are not limited to, for example, RT-PCR analysis, hybridization based analysis (i.e., Northern analysis), spectophotometry and/or proteomic analysis (i.e., mass spectral analysis).

More sophisticated techniques for assaying for secondary modification of proteins (for example, phosphorylation, acetylation, farnesylation, etc.) and the effect thereof on the activity of such modified proteins are known in the art (for example, immunoblotting, yeast-2-hybrid assays, reporter-based assays, activity assays, etc.).

The instant invention also relates to a method of studying clinical behavior of a tumor comprising
(a) generating a neoplastic signature profile of said tumor which comprises one or more proteins which are differentially expressed in tumor versus non-tumor tissues in accordance with the forgoing and
(b) comparing said signature profile with a cancer dataset, for example, one containing cancer tissue from a patient or many patients with clinical outcomes and progression.

Examples of such datasets are known in the art, for example, hepatocellular carcinoma proteome database of Biotechnology Processing Center, Singapore (available on the world-wide-web at bti.a-star.edu.sg/hccm/servlet/CounterDB). Other datasets may also be employed. In one embodiment, the clinical behavior of a tumor relates to the probability of metastasis of said tumor. In another embodiment, the clinical behavior relates to probability of survival associated with said tumor or progression free survival. The evaluation of clinical outcome may be drawn to a predictive analysis of overall survival or a predictive analysis of metastasis-free survival. Other classification parameters, for example, tumor differentiation, tumor size, tumor grade, and/or staging methods may also be used.

In one embodiment, as a result of such dataset comparison, clinical behavior of a tumor in relation to progression and/or metastasis can be studied. Thus, there is provided a means for studying the progression of cancer and/or differentiating non-metastatic from metastatic disease. For instance, the invention provides a method for predicting the outcome (for example, progressive or metastatic nature) of HCC in a patient comprising detecting, in a test sample of said patient, the level of expression at least one biomarker which is s-c-Kit, HGF, Ras p21, VEGF, s-VEGFR-2, s-VEGFR-3, pERK, Ang2, bFGF, or IGF-2; and comparing said level of expression of said biomarker with a reference standard which comprises dataset measurements of the expression levels of said biomarker in HCC patients, wherein association of said biomarker with progressive or metastatic HCC in said dataset is indicative of the outcome of said HCC in said patient. Using the aforementioned techniques, an association of the aforementioned expression profile with progression and/or metastasis can be calibrated based on information obtained from the datasets.

### DETAILED DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a protein” is a reference to one or more proteins and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein, including the disclosures in U.S. Patent Publication Nos. 20070178494 and 20070105142, are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### DEFINITIONS

For convenience, the meaning of other terms and phrases employed in the specification, examples, and appended claims are provided below.

“And” as used herein is interchangeably used with “or” unless expressly stated otherwise.

By “large differential expression” it is meant that the level of expression is significantly (e.g. p≦0.05) or differs by at least about 20%, more preferably, 50%, most preferably 100% based on the level of expression in a reference standard.

As used herein, the terms “cancer” or “tumor” includes, but is not limited to, solid tumors, such as cancers of the breast, respiratory tract, brain, reproductive organs, digestive tract, urinary tract, eye, liver, kidney, skin, head and neck, thyroid, parathyroid, and their distant metastases. The terms also include lymphomas, sarcomas, and leukemias.

Preferred cancers include, but are not limited to, liver cancers (both primary and secondary). Primary liver cancers include benign tumors as well as malignant tumors of the liver. Examples of benign liver tumors include, but are not limited to, hemangiomas, hepatic adenomas and focal nodular hyperplasia (FNH). Malignant liver tumors include, but are not limited to, hepatocellular carcinoma (HCC), cholangiocarcinomas, angiosarcomas, hemangiosarcomas as well as hepatoblastoma. Secondary (metastatic) liver cancer comprises cancer cells that have spread to a liver from a primary tumor at a separate site. In such instances, the tumor could comprise cancer cells of colon, rectum, stomach, breasts and/or lungs.

Particularly studied cancers herein are hepatocellular carcinomas (HCC). Examples of HCC include, but are not limited to, fibrolamellar, pseudoglandular (adenoid), pleomorphic (giant cell) and clear cell HCC.

“Hepatocellular carcinoma” (HCC, also called hepatoma), as used herein, is a primary malignancy (cancer) of the liver.

Preferably, the methods of the present invention are useful in the detection, prognostication and guidance for the treatment of patients with advanced hepatocellular carcinoma (HCC). Staging procedures for the characterization of HCC patients in various stages, for example, asymptomatic, advanced, etc. are known in the art.

The phrase “cancer type” (or simply “type”) as used herein refers to a diagnostic classification of a cancer. For example, with respect to liver cancers, the phrase may refer to a broad class (e.g., hepatocellular carcinoma, cholangiocarcinomas, angiosarcomas, hemangiosarcoma, hepatoblastoma, etc.) or to a subtype or subgroup falling within a class (e.g., fibrolamellar HCC, pseudoglandular HCC, pleomorphic HCC and clear cell HCC).

A “sample” may be of any biological tissue or fluid or cells from any organism as well as cells raised in vitro, such as cell lines and tissue culture cells. Preferably, the “sample” comprises a biological specimen isolated from a patient suffering from a neoplastic disease (i.e., a “clinical sample”) and/or healthy human subjects. Such sample may comprise a specimen into which biomarkers are directly released, or a specimen into which biomarkers are captured. Such derivation may occur either in vivo or in vitro. In some instances, the biological specimen is a circulating fluid such as blood or lymph, or a fraction thereof, such as serum or plasma. In other cases, the biological specimen remains substantially in a particular locus, for example, synovial fluid, cerebrospinal fluid or interstitial fluid. In still further cases, the biological specimen is an excreted fluid, for example, urine, breast milk, saliva, sweat, tears, mucous, nipple aspirants, semen, vaginal fluid, pre-ejaculate and the like. A biological specimen also refers to a liquid in which cells are cultured in vitro such as a growth medium, or a liquid in which a cell sample is homogenized, such as a buffer. The specimen may further comprise swabs comprising tissue, biopsied tissue, tissue sections, cultured cells, surgically resected tumor sample, etc. “Samples” may also include sections of tissues, such as frozen sections or formalin fixed sections taken for histological purposes.

The term “patient” or “subject” as used herein includes mammals (e.g., humans and animals).

Protein Samples

Any sample from any source can be used with the disclosed method. In general, “protein samples” should be samples that contain, or may contain, protein molecules. Examples of suitable protein samples include cell samples, tissue samples, cell extracts, components or fractions purified from another sample, environmental samples, biofilm samples, culture samples, tissue samples, bodily fluids, and biopsy samples. Numerous other sources of samples are known or can be developed and any can be used with the disclosed method. Preferred protein samples for use with the disclosed method are samples of cells and tissues. Protein samples can be complex, simple, or anywhere in between. For example, a protein sample may include a complex mixture of proteins (a tissue sample, for example), a protein sample may be a highly purified protein preparation, or a single type of protein.

The “reference standard” can be any number of types of samples or method of determining a reference expression level for each protein, including normal plasma, serum, tissue or cells, the normal range from normal plasma, serum, or tissue, the range of expression within a group of patients, or a set of patients with a certain outcome. By “reference standard” it is meant a sample which provides a baseline for the assayed parameter (i.e., a control). Reference standards may comprise normal or non-cancerous cell/tissue sample isolated from a subject as well as cultured primary cells/tissues. Examples of reference standard include, but are not limited to, adjacent normal cells/tissues obtained from the same organ or body location of a patient, a sample isolated from a normal subject, a primary cells/tissues obtained from a depository (for example, American type tissue culture Accession No.: 87253 or 87254, which relate to human embryonic liver at 72 days and 58 days, respectively), etc. A reference standard can also be the expression level for a set of patients, such as a set of (e.g.) HCC patients, or for the set of HCC patients receiving a certain treatment (e.g. sorafenib) or for a set of patients with one outcome versus another outcome. In the former case the specific level of each patient can be assigned to a percentile level of expression, or expressed as either higher or lower than the mean or average. The term “reference standard” as used herein particularly includes normal cells, cells from patients treated with standard chemotherapy, for example, sorafenib or cells from patients having benign lymphoma. A reference standard may also comprise a measured value for example, average/median level of expression of a particular gene in a population. Such a population may comprise normal subjects, patients with HCC who have not undergone any treatment (i.e., treatment naïve), HCC patients undergoing sorafenib therapy, HCC patients undergoing chemotherapy other than sorafenib or patients having benign liver cancer. A “positive reference standard” or “positive control” as is known in the art, comprising, for example, transformed heptocellular carcinoma cell-line (HepG2 cells; ATCC No. HB-8065) may be optionally employed.

In particularly preferred embodiments, the reference standard comprises a sample which is of the same lineage and/or type as the test sample. In such embodiments, both the test sample and reference standard comprise blood sample (for plasma biomarkers) and/or tumor sample (for tumor biomarkers).

An “address” on an array (e.g., a microarray) refers to a location at which an element, for example, an oligonucleotide, is attached to the solid surface of the array.

The terms “array” or “matrix” refer to an arrangement of addressable locations or “addresses” on a device. The locations can be arranged in two-dimensional arrays, three-dimensional arrays, or other matrix formats. The number of locations may range from several to at least hundreds of thousands. Most importantly, each location represents a totally independent reaction site. A “nucleic acid array” refers to an array containing nucleic acid probes, such as oligonucleotides or larger portions of genes. The nucleic acid on the array is preferably single-stranded. Arrays wherein the probes are oligonucleotides are referred to as “oligonucleotide arrays” or “oligonucleotide chips.” An “antibody array” refers to an array containing antibody molecules that are capable of binding to one or more antigens (i.e., proteins). A “microarray,” also referred to herein as a “biochip” or “biological chip,” is an array of regions having a density of discrete regions of at least about 100/cm², and preferably at least about 1000/cm². The regions in a microarray have typical dimensions, for example, diameters, in the range of between about 10-250 μm, and are separated from other regions in the array by about the same distance.

“Biological activity” or “bioactivity” or “activity” or “biological function,” which are used interchangeably, herein mean an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The term “biological sample,” as used herein, refers to a sample obtained from an organism or from components (e.g., cells) of an organism. The sample may be of any biological tissue or fluid. The sample may be a sample which is derived from a patient. Such samples include, but are not limited to, sputum, blood, blood cells (e.g., white cells), tissue or biopsy samples (e.g., tumor biopsy), urine, peritoneal fluid, and pleural fluid, or cells therefrom. Biological samples may also include sections of tissues such as frozen sections taken for histological purposes.

The term “gene” refers to a nucleic acid sequence that comprises control and coding sequences necessary for the production of a polypeptide or precursor. The polypeptide can be encoded by a full length coding sequence or by any portion of the coding sequence. The gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions which could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

As used herein, the term “nucleic acid” refers to polynucleotides such as deoxyribonucleic acid (DNA) and, where appropriate, ribonucleic acid (RNA). The term should also be understood to include, as equivalents, analogs of either RNA or DNA made from nucleotide analogs and, as applicable to the embodiment being described, single-stranded (sense or antisense) and double-stranded polynucleotides. Chromosomes, cDNAs, mRNAs, rRNAs, and ESTs are representative examples of molecules that may be referred to as nucleic acids.

The term “oligonucleotide” as used herein refers to a nucleic acid molecule comprising, for example, from about 10 to about 1000 nucleotides. Oligonucleotides for use in the present invention are preferably from about 15 to about 150 nucleotides, more preferably from about 20 to about 100 in length. The oligonucleotide may be a naturally occurring oligonucleotide or a synthetic oligonucleotide. Oligonucleotides may be prepared by the phosphoramidite method (Beaucage and Carruthers, Tetrahedron Lett. 22:1859-62, 1981), or by the triester method (Matteucci, et al., J. Am. Chem. Soc. 103:3185, 1981), or by other chemical methods known in the art.

The term “specific hybridization” of a probe to a target site of a template nucleic acid refers to hybridization of the probe predominantly to the target, such that the hybridization signal can be clearly interpreted. As further described herein, such conditions resulting in specific hybridization vary depending on the length of the region of homology, the GC content of the region, and the melting temperature (“Tm”) of the hybrid. Thus, hybridization conditions may vary in salt content, acidity, and temperature of the hybridization solution and the washes.

The term “isolated,” as used herein, with respect to nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, culture medium when produced by recombinant DNA techniques, or chemical precursors or other chemicals when chemically synthesized. Moreover, an “isolated nucleic acid” may include nucleic acid fragments which are not naturally occurring as fragments and would not be found in the natural state. The term “isolated” is also used herein to refer to polypeptides which are isolated from other cellular proteins and is meant to include both purified and recombinant polypeptides.

As used herein, the terms “label” and “detectable label” refer to a molecule capable of detection, including, but not limited to, radioactive isotopes, fluorophores, chemiluminescent moieties, enzymes, enzyme substrates, enzyme cofactors, enzyme inhibitors, dyes, metal ions, ligands (e.g., biotin or haptens), and the like. The term “fluorescer” refers to a substance or a portion thereof which is capable of exhibiting fluorescence in the detectable range. Particular examples of labels which may be used in the present invention include fluorescein, rhodamine, dansyl, umbelliferone, Texas red, luminol, NADPH, alpha-beta-galactosidase, and horseradish peroxidase.

As used herein, the term “level of expression” refers to the measurable expression level of a given nucleic acid. The level of expression of a nucleic acid is determined by methods well known in the art. The term “differentially expressed” or “differential expression” refers to an increase or decrease in the measurable expression level of a given nucleic acid. As used herein, “differentially expressed” or “differential expression” means the difference in the level of expression of a protein is significant (e.g. p≦0.05), which can be at least a 1.2-fold, at least 1.4-fold, at least 2.0-fold or more in two samples used for comparison, both of which are compared to the same control protein (for example, actin) and then subsequently to a reference standard. “Differentially expressed” or “differential expression” according to the invention also means a 1.2-fold, or more, up to and including 1.5-fold, 2-fold, 5-fold, 10-fold, 20-fold, 50-fold or more difference in the level of expression of a protein in two samples used for comparison. A protein is also said to be “differentially expressed” in two samples if one of the two samples contains no detectable expression of a given nucleic acid, provided that the detectably expressed nucleic acid is expressed at +/− at least 1.2 fold. Differential expression of a protein is “inhibited” if the difference in the level of expression of the protein in two or more samples used for comparison is altered such that it is no longer at least a 1.2 fold difference. Absolute quantification of the level of expression of a protein may be accomplished by including a known concentration(s) of one or more control proteins, generating a standard curve based on the amount of the control proteins and extrapolating the expression level of the “unknown” protein species from the signal intensities of the unknown with respect to the standard curve (for example, optical density based assays).

As used herein, the phrase “detecting the level expression” includes methods that quantitate expression levels as well as methods that determine whether a protein of interest is expressed at all. Thus, an assay which provides a yes or no result without necessarily providing quantification of an amount of expression is an assay that requires “detecting the level of expression” as that phrase is used herein. The proteins identified as being differentially expressed in liver cancer may be used in a variety of proteomic assays to detect or quantititate the expression level of a proteins or multiple proteins in a given sample. For example, traditional antibody-based assays, 2D gel electrophoresis, ELISA assays, and the like. For differentially expressed genes, Northern blotting, nuclease protection, RT-PCR, in situ hybridization, sequencing, and differential display methods may be used. Those methods are useful for some embodiments of the invention. However, methods and assays of the invention are most efficiently designed with antibody array or chip-based methods.

Proteins

The term “protein” is used interchangeably herein with the terms “peptide” and “polypeptide.”

Variant

A “variant” of polypeptide refers to a polypeptide having an amino acid sequence in which one or more amino acid residues is altered. The variant may have “conservative” changes, wherein a substituted amino acid has similar structural or chemical properties (e.g., replacement of leucine with isoleucine). A variant may also have “nonconservative” changes (e.g., replacement of glycine with tryptophan). Analogous minor variations may include amino acid deletions or insertions, or both. Guidance in determining which amino acid residues may be substituted, inserted, or deleted without abolishing biological or immunological activity may be identified using computer programs well known in the art, for example, LASERGENE software (DNASTAR). The term “variant,” when used in the context of a polynucleotide sequence, may encompass a polynucleotide sequence related to that of a particular gene or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of for example, a certain population, a disease state, or a propensity for a disease state.

The term “expression profile,” which is used interchangeably herein with “protein expression profile” and “proteome” or proteomic signature of a cell refers to a set of values representing levels or activity of one or more proteins. An expression profile preferably comprises values representing expression levels of at least about two proteins, preferably at least about 2, 3, 5, 6 or more proteins. Expression profiles may also comprise a level of a protein which is expressed at similar levels in multiple cells and conditions (e.g., actin). For example, an expression profile of a diseased cell of cancer refers to a set of values representing protein levels of at least one control protein and 2 to 6 or more of the proteins in a diseased cell or tissue.

An expression profile in one cell is “similar” to an expression profile in another cell when the level of expression of the proteins in the two profiles are sufficiently similar that the similarity is indicative of a common characteristic, for example, the same type of cell. Accordingly, the expression profiles of a first cell and a second cell are similar when at least 75% of the proteins that are expressed in the first cell are expressed in the second cell at a level that is within a factor of two relative to the first cell.

“Bind(s) specifically” not only refers to interaction between an antibody and a target protein of the present invention, but also with other molecules, such as, for example, proteins, aptamers, and the like. As is known in the art, the antigen-antibody “specific binding” embraces minor changes outside the epitope region that can be still be detected by an antibody directed thereto.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

Signatures

The present invention relates to one or more protein biomarkers selected from the group consisting of VEGF, s-VEGFR-2, VEGFR-3, s-c-Kit, HGF, Ras p 21, pERK, Ang2, bFGF, or IGF-2, which make up a “proteomic signature.” Such signature may comprise a single protein or a combination of 2, preferably 3, more preferably 4, particularly preferably 5 and most preferably 6 or more of the aforementioned proteins. Non-limiting examples of such combinations include, but are not limited to, HGF and VEGF; HGF and s-VEGFR-3; VEGF and s-VEGFR-3; HGF, VEGF and s-VEGFR-3; HGF and Ras p21; HGF, VEGF and Ras p21; VEGF and Ras p21; s-VEGFR-3 and Ras p21; c-KIT and bFGF; c-KIT and IGF-2; bFGF and IGF-2; HGF and bFGF; HGF and IGF-2, etc. Signatures comprising a combination of HGF, s-c-Kit, bFGF and/or IGF-2 are most preferred.

Signatures of the present invention may comprise genes encoding on or more of the aforementioned protein biomarkers, for example, VEGF, s-VEGFR-2, VEGFR-3, s-c-Kit, HGF, Ras p 21, pERK, Ang2, bFGF, or IGF-2. Such are described herein as “gene signatures.” As described hereinbefore, such gene signatures may comprise Such signature may comprise a single gene or a combination of 2, preferably 3, more preferably 4, particularly preferably 5 and most preferably 6 of the aforementioned genes.

To facilitate the understanding of such combinations, the aforementioned biomarkers are grouped as follows:

Group A comprising HGF, s-c-Kit, s-VEGFR-3 and Ang2;

Group B comprising VEGF, s-VEGFR-2, Ras p21

Preferred combinations include, but are not limited to:

(a) Combinations comprising one biomarker from Group A and one biomarker from Group B

(i) HGF and VEGF;

(ii) s-c-Kit and VEGF;

(iii) s-VEGFR-3 and VEGF;

(iv) HGF and s-VEGFR-2;

(v) s-c-Kit and s-VEGFR-2;

(vi) s-VEGFR-3 and s-VEGFR-2;

(vii) Ang2 and VEGF;

(viii) Ang2 and sVEGFR2;

(ix) Ang2 and Ras p 21;

(x) IGF-2 and VEGF;

(xi) IGF-2 and sVEGFR2;

(xii) IGF-2 and Ras p21; or

(b) Combinations comprising one biomarker from Group A and two biomarkers from Group B

(i) HGF and VEGF plus s-VEGFR-2;

(ii) s-c-Kit and VEGF plus s-VEGFR-2;

(iii) s-VEGFR-3 and VEGF plus s-VEGFR-2;

(iv) Ang2 and VEGF plus sVEGFR2;

(v) Ang2 and sVEGFR2 plus Ras p21;

(vi) Ang2 and Ras p21 plus VEGF;

(vii) IGF-2 VEGF and sVEGFR2;

(viii) IGF-2, sVEGFR2 and Ras p21;

(ix) IGF-2, VEGF and Ras p21; or

(c) Combinations comprising two biomarkers from Group A and one biomarker from Group B

(i) HGF, s-c-Kit and VEGF;

(ii) HGF, s-c-Kit and s-VEGFR-2;

(iii) HGF, s-VEGFR-3 and VEGF;

(iv) HGF, s-VEGFR-3 and s-VEGFR-2;

(v) s-c-Kit, s-VEGFR-3 and VEGF;

(vi) s-c-Kit, s-VEGFR-3 and s-VEGFR-2;

(vii) HGF, Ang2 and VEGF;

(viii) HGF, Ang2 and s-VEGFR-2;

(ix) s-c-Kit, Ang2 and VEGF;

(x) s-c-Kit, s-VEGFR-3 and s-VEGFR-2;

(xi) s-VEGFR-3, Ang2 and VEGF;

(xii) s-VEGFR-3, Ang2 and s-VEGFR-2;

(xiii) IGF-2, HGF and VEGF;

(xiv) IGF-2, HGF and sVEGFR2;

(xv) IGF-2, HGF and Ras p21;

(xvi) IGF-2, Ang2 and VEGF;

(xvii) IGF-2, Ang2 and sVEGFR2;

(xviii) IGF-2, Ang2 and Ras p21;

(xix) IGF-2, s-c-Kit and VEGF;

(xx) IGF-2, s-c-Kit and sVEGFR2;

(xxi) IGF-2, s-c-Kit and Ras p21; or

(d) Combinations comprising two biomarkers from Group A and two biomarkers from Group B

(i) HGF, s-c-Kit and VEGF plus s-VEGFR-2;

(ii) HGF, s-VEGFR-3 and VEGF plus s-VEGFR-2;

(iii) s-c-Kit, s-VEGFR-3 and VEGF plus s-VEGFR-2;

(iv) HGF, Ang2 and VEGF plus s-VEGFR-2;

(v) s-c-Kit, Ang2 and VEGF plus s-VEGFR-2;

(vi) s-VEGFR-3, Ang2 and VEGF plus s-VEGFR-2;

(vii) IGF-2, HGF and VEGF plus sVEGFR2;

(viii) IGF-2, HGF and sVEGFR2 plus Ras p21;

(ix) IGF-2, HGF and VEGF plus Ras p21;

(x) IGF-2, Ang2 and VEGF plus sVEGFR2;

(xi) IGF-2, Ang2 and sVEGFR2 plus Ras p21;

(xii) IGF-2, Ang2 and VEGF plus Ras p21;

(xiii) IGF-2, s-c-Kit VEGF plus sVEGFR2;

(xiv) IGF-2, s-c-Kit and sVEGFR2 plus Ras p21;

(xv) IGF-2, s-c-Kit and VEGF plus Ras p21; or

(e) Combinations comprising three biomarkers from Group A and one biomarker from Group B

(i) HGF, s-c-Kit, s-VEGFR-3 and VEGF;

(ii) HGF, s-c-Kit, s-VEGFR-3 and s-VEGFR-2;

(iii) HGF, s-c-Kit, Ang2 and VEGF;

(iv) HGF, s-c-Kit, Ang2 and s-VEGFR-2;

(vi) s-c-Kit, s-VEGFR-3, Ang2 and VEGF;

(vi) s-c-Kit, s-VEGFR-3, Ang2 and s-VEGFR-2;

(vii) HGF, s-VEGFR-3, Ang2 and VEGF;

(viii) HGF, s-VEGFR-3, Ang2 and s-VEGFR-2;

(ix) HGF, s-c-Kit, IGF-2 and VEGF;

(x) HGF, s-c-Kit, IGF-2 and s-VEGFR-2;

(xi) HGF, IGF-2, Ang2 and VEGF;

(xii) HGF, IGF-2, Ang2 and s-VEGFR-2; or

(f) Combination comprising three biomarkers from Group A and two biomarkers from Group B

(i) HGF, s-c-Kit, s-VEGFR-3 and VEGF plus s-VEGFR-2;

(ii) HGF, s-c-Kit, Ang2 and VEGF plus s-VEGFR-2;

(iii) HGF, Ang2, s-VEGFR-3 and VEGF plus s-VEGFR-2;

(iv) s-c-Kit, s-VEGFR-3, Ang2 and VEGF plus s-VEGFR-2;

(v) HGF, s-c-Kit, IGF-2 and VEGF plus s-VEGFR-2;

(vi) HGF, s-c-Kit, IGF-2 and VEGF plus s-VEGFR-2;

(vii) HGF, IGF-2, Ang2 and VEGF plus s-VEGFR-2;

(viii) HGF, IGF-2, Ang2 and VEGF plus s-VEGFR-2; or

(g) Combination comprising four biomarkers from Group A and one biomarker from Group B

(i) HGF, s-c-Kit, s-VEGFR-3, Ang2 and VEGF;

(ii) HGF, s-c-Kit, s-VEGFR-3, Ang2 and s-VEGFR-2;

(iii) HGF, s-c-Kit, IGF-2, Ang2 and VEGF;

(iv) HGF, s-c-Kit, IGF-2, Ang2 and s-VEGFR-2; or

(h) Combination comprising four biomarkers from Group A and two biomarkers from Group B

(i) HGF, s-c-Kit, s-VEGFR-3, Ang2 and VEGF plus s-VEGFR-2;

(ii) HGF, s-c-Kit, IGF-2, Ang2 and VEGF plus s-VEGFR-2; or

(i) Combinations comprising all of the aforementioned biomarkers;

Antibodies

The term “antibody,” as used herein, is intended to include whole antibodies, for example, of any isotype (IgG, IgA, IgM, IgE, etc.), and includes fragments thereof which are also specifically reactive with a vertebrate (e.g., mammalian) protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

Biomarker

The term “biomarker” or “marker” encompasses a broad range of intra- and extra-cellular events as well as whole-organism physiological changes. Biomarkers may represent essentially any aspect of cell function, for example, but not limited to, levels or rate of production of signaling molecules, transcription factors, metabolites, gene transcripts as well as post-translational modifications of proteins. Biomarkers may include whole genome analysis of transcript levels or whole proteome analysis of protein levels and/or modifications.

Preferably the biomarkers of the present invention are proteins and/or polypeptides.

A biomarker may also refer to a gene or gene product which is up-regulated or down-regulated in a compound-treated, diseased cell or tissue of a subject having the disease compared to an untreated diseased cell or tissue or compared to patients with the same disease, or treated patients with different outcomes. That is, the gene or gene product is sufficiently specific to the treated cell or tissue that it may be used, optionally with other genes or gene products, to identify, predict, or detect efficacy of a small molecule or any therapy and/or clinical outcome for the patient. Thus, a biomarker is a gene or gene product that is characteristic of efficacy of a compound in a diseased cell or the response of that diseased cell to treatment by the compound.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA. Assays and methods of the invention may utilize available formats to simultaneously screen at least about 2, 10, 100, 10,000, or 1,000,000 or more, and preferably about 2 to 50 or more different nucleic acid hybridizations.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide. For instance, high stringency conditions can be achieved by incubating the blot overnight (e.g., at least 12 hours) with a polynucleotide probe in a hybridization solution containing, e.g., about 5×SSC, 0.5% SDS, 100 μg/ml denatured salmon sperm DNA and 50% formamide, at 42° C., or hybridizing at 42° C. in 5×SSPE, 0.5% SDS, and 50% formamide, 100 pg/ml denatured salmon sperm DNA, and washing at 65° C. in 0.1% SSC and 0.1% SDS. Blots can be washed at high stringency conditions that allow, e.g., for less than 5% base-pair mismatch (e.g., wash twice in 0.1% SSC and 0.1% SDS for 30 min at 65° C.), e.g., selecting sequences having 95% or greater sequence identity.

Hybridization based assays and methods employed therein are known in the art. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 pg/ml denatured salmon sperm DNA, 5×Denhardt's solution, and 50% formamide), at 22-68° C., overnight, and then hybridized with a detectable polynucleotide probe under conditions appropriate to achieve the desired stringency. In general, when high homology or sequence identity is desired, a high temperature can be used (e.g., 65° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formamide can be omitted. See, e.g., Current Protocols in Molecular Biology, Chapter 6, Screening of Recombinant Libraries; Sambrook et al., Molecular Cloning, 1989, Chapter 9.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical monomer unit (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

“Homology” or “identity” may be determined by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268 and Altschul, (1993) J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (1994) Nature Genet. 6, 119-129) which is filly incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) Proc. Natl. Acad. Sci. USA 89, 10915-10919, fully incorporated by reference). Four blastn parameters were adjusted as follows: Q=10 (gap creation penalty); R=10 (gap extension penalty); wink=1 (generates word hits at every position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

Probes

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, locked nucleic acids, PNA's, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages. When an array contains several probes corresponding to one gene, these probes are referred to as a “gene-probe set.” A gene-probe set may consist of for example, about 2 to about 20 probes, preferably from about 2 to about 10 probes, particularly preferably from about 4 to about 8 probes and most preferably about 5 probes.

Kits

The invention further relates to “kits” combining, in different combinations, high-density antibody arrays, reagents for use with the arrays, signal detection and array-processing instruments, proteomic databases and analysis, manuals and database management software described above. The kits may be used, for example, to predict or model the toxic response of a test compound, to monitor the progression of liver disease states, to identify genes that show promise as new drug targets and to screen known and newly designed drugs as discussed above. The databases packaged with the kits are a compilation of expression patterns from human or laboratory animal proteomes and/or fragments (corresponding to the proteins of the present invention). Data is collected from a repository of both normal and diseased animal tissues and provides reproducible, quantitative results, i.e., the degree to which a protein is over-expressed or under-expressed compared to a reference standard under a given condition.

Kits can also include those for PCR, sequencing, in situ hybridization.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Oligonucleotide probe arrays for expression monitoring can be made and used according to any techniques known in the art (see for example, Lockhart et al., (1996) Nat. Biotechnol. 14, 1675-1680; McGall et al., (1996) Proc. Nat. Acad. Sci. USA 93, 13555-13460). Such probe arrays may contain at least one or more oligonucleotides that are complementary to or hybridize to one or more of the genes described herein. Such arrays may also contain oligonucleotides that are complementary or hybridize to at least about 2, 3, 4, 5, 6, or more the genes described herein.

The measurement of protein using aptamers, or other probes, are also permissible with the instant invention. The instant invention also relates to measurement of proteins and oligonucleotides simultaneously using appropriate probes. In yet another aspect, the instant invention relates to the hybridization (or binding) of probes to insoluble proteins (such as in FFPE samples), and then removal and measurement of the probe, or probe/target molecule, even where the target molecule may be damaged, fractured or cleaved, but the probe or probe complex is intact or held together sufficiently. Any method where the probe associates with both cross-linked or surface bound target molecule (e.g. membrane bound receptors) and soluble target molecule (for example, soluble receptor variants), or associated only with the cross-linked or surface bound target molecule, is reduced to an analyzable amount relative to the target molecule, then removed from the tissue and measured.

Databases

The present invention includes relational databases containing proteomic information, for instance for the hereinbefore described proteins, as well as expression information relating thereto in various cell or tissue samples. The expression pattern may be associated with patient treatment and response or outcome information or other diagnostic information (such as determination of disease stage, e.g. HCC) or patient risk assessment (by e.g., IPI score). Databases may also contain information associated with a given proteome or tissue sample such as descriptive information about the protein associated with the sequence information, or descriptive information concerning the clinical status of the biological sample, or the patient from which the sample was derived. The database may be designed to include different parts, for instance a proteome database and a gene expression database. Methods for the configuration and construction of such databases are widely available. The databases of the invention may be linked to an outside or external database. Examples of such external databases include, but are not limited to, Genome Medicine Database of Japan (available on the world-wide-web at gemdbj.nibio.go.jp/dgdb/). The databases of the invention may be used to produce, among other things, electronic Western blots to allow the user to determine the cell type or tissue in which a given protein is expressed and to allow determination of the abundance or expression level of a given protein in a particular tissue or cell.

The databases of the invention may also be used to present information identifying the expression level in a tissue or cell of a set of genes comprising at least two of the aforementioned proteins comprising the step of comparing the expression level of at least proteins in the tissue to the level of expression of the proteins in the database. Such methods may be used to predict the physiological state of a given tissue by comparing the level of expression of a protein or proteins from a sample to the expression levels found in a normal tissue, a cancerous tissue, or a malignant tumor or the tissue of patients with the same disease (e.g. HCC) and treatment (e.g. sorafenib) or other patients with a different clinical outcome. Such methods may also be used in the drug or agent screening assays as described above.

Any appropriate computer platform may be used to perform the necessary comparisons between expression information, post-translational modification information (for example, splicing, phosphorylation), activity information and any other information in the database or provided as an input. For example, a large number of computer workstations are available from a variety of manufacturers, such has those available from Silicon Graphics. Client-server environments, database servers and networks are also widely available and appropriate platforms for the databases of the invention.

Prognostication

By “outcome” it is meant evaluation of time to progression (TTP) and/or overall survival (OS) or progression free survival. Techniques and methodology for predicting clinical outcomes and risk, for example, calculation of international prognostication index (IPI) to assign risk are known in the art.

Overall survival (OS) is defined as the time from randomization to death due to any cause. Overall survival (OS) of subjects alive at the time of analysis will be censored at their last date of follow-up.

Symptomatic progression is defined as a decrease of at least 4 points from baseline score based on the FHSI-8 questionnaire, confirmed at the following 3 week scheduled assessment. Death will not be considered as symptomatic progression except when there is a decrease in score on the FHSI-8 of 4 points or more from baseline followed by death prior to the next scheduled visit. If the reason for withdrawal from the study is deterioration to an ECOG 4 status, this will be considered as symptomatic progression.

Time to symptomatic progression (TTSP) is defined as the time from randomization to the first documented symptomatic progression (see above for the definition of symptomatic progression). For subjects who had not progressed symptomatically at the time of analysis, TTSP will be censored at their last date of FHSI-8 assessment.

Time to progression (TTP) is defined as the time from randomization to disease progression (radiological only). Patients without tumor progression at the time of analysis will be censored at their last date of tumor evaluation.

Disease control rate is defined as the proportion of patients who have a best response rating of Complete Response (CR), Partial Response (PR) or Stable Disease (SD) according to RECIST that is maintained for at least 28 days from the first demonstration of that rating.

Best overall response rate is defined as the proportion of patients with the best tumor response (confirmed partial or complete response) that is achieved during treatment or within 30 days after termination of active therapy that is confirmed according to the RECIST tumor response criteria.

Overall response duration will be measured from the date of first objective response to the date that PD is first objectively documented or death (if death occurs earlier than progression). For subjects failing to achieve an objective response, overall response duration will be assigned value zero.

Time to objective response is defined as the time from the date of randomization until the date that an objective tumor response is first documented according to the RECIST tumor response criteria. Response must subsequently be confirmed. For subjects failing to achieve an objective response and did not progress during the trial, time to objective response will be censored at their last date of tumor evaluation. For subjects who have PD as their best response, time to objective response will be assigned value infinite.

Recurrence Free Survival (RFS) is defined as the time from randomization to the first documented disease recurrence by independent radiological assessment or death due to any cause whichever occurs first. For subjects who had not recurred or died at the time of analysis, RFS will be censored at their last date of evaluable scan.

Disease recurrence (intrahepatic or extrahepatic) is defined as follows:

Intrahepatic recurrence is defined as appearance of one or more intrahepatic lesions fulfilling the following conditions:

1. Its longest diameter is larger than or equal to 10 mm and the nodule shows the typical vascular pattern of HCC on dynamic imaging, i.e. hypervascularization in the arterial phase with wash-out in the portal venous or late venous phase (one imaging technique).

2. Lesions larger than 10 mm that do not show a typical vascular pattern can be diagnosed as HCC by evidence of at least 1 cm interval growth in subsequent scans.

Extrahepatic recurrence is defined as per RECIST criteria. Removal due to ascites or pleural effusion, only if proven malignant.

Time to recurrence (TTR) is defined as the time from randomization to the first documented disease recurrence by independent radiological assessment. For subjects who had not recurred at the time of analysis, TTR will be censored at their last date of evaluable scan.

In certain methods described herein, an individual who is at risk for poor prognosis and/or outcome is an individual in whom one or more proteins selected from the group consisting of VEGF, s-VEGFR-2, VEGFR-3, s-c-Kit, HGF, Ras p 21, pERK, Ang2, bFGF, or IGF-2 are differentially expressed. In other embodiment a combination of genes may be used. The significance associated with gene is measured by techniques known in the art. For example, significance may be measured with calculation of odds ratio. In a further embodiment, the significance is measured by a statistical analysis (for example, survival curve analysis).

In one embodiment, a significant risk is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors such as family history of cancer, particularly, familial history of HCC, cigarette smoking, alcohol consumption, liver cirrhosis, lack of physical activity, viral infection (for example, hepatitis virus infection) and inflammatory components as reflected by known inflammatory markers.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

Aspects of the instant invention include, but are not limited to:

In one embodiment, the present invention provides for the following aspects

Aspect 1. A method of prognosticating the outcome of a patient suffering from hepatocellular carcinoma (HCC), comprising

detecting, in a test sample of said patient, the expression levels of at least one biomarker which is vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin 2 (Ang2), basic fibroblast growth factor (bFGF) or insulin-like growth factor (IGF); and

comparing said level of expression of said biomarker in said patient test sample with a reference standard,

wherein differential levels of expression of said biomarker in said test sample compared to said reference standard is indicative of said outcome.

Aspect 2. The method according to claim 1, wherein the biomarker is a protein.
Aspect 3. The method according to aspect 2, wherein said level of expression of said biomarker in said test sample is increased or decreased compared to said reference standard.
Aspect 4. The method according to aspect 2, wherein said biomarker is plasma HGF, VEGF, s-VEGFR-3, Ras p21, Ang2, bFGF, IGF-2 or a combination thereof.
Aspect 5. The method according to aspect 2, wherein said outcome is overall survival (OS) and/or time to progression (TTP).
Aspect 6. The method according to aspect 2, wherein said biomarker is HGF, VEGF, s-VEGFR-3, Ang2, IGF-2 and said outcome is overall survival (OS).
Aspect 7. The method according to aspect 6, wherein

attenuation of said HGF, VEGF, s-VEGFR-3, or Ang2 levels in said HCC patient compared to said reference standard; or

elevation of said IGF-2 in said HCC patient compared to said reference standard is indicative of improved overall survival (OS).

Aspect 8. The method according to aspect 6, wherein

elevation of said HGF, VEGF, s-VEGFR-3, or Ang2 levels in said HCC patient compared to said reference standard; or

attenuation of said IGF-2 in said HCC patient compared to said reference standard is indicative of worse overall survival.

Aspect 9. The method according to aspect 2, wherein said biomarker is VEGF, Ras p21, Ang2 and said outcome is time to progression (TTP).
Aspect 10. The method according to aspect 9, wherein attenuation of said VEGF levels, attenuation of said Ang2 levels, or elevation of Ras p21 levels in said HCC patient compared to said reference standard is indicative of longer time to progression (TTP).
Aspect 11. The method according to aspect 9, wherein elevation of said VEGF levels, elevation of said Ang2 levels, or attenuation of said Ras p21 levels in said HCC patient compared to said reference standard is indicative of shorter time to progression (TTP).
Aspect 12. The method according to aspect 4, wherein
said biomarker is plasma HGF, VEGF, s-VEGFR-3, Ang2, bFGF, or IGF-2; and
said reference standard comprises 75^{th} percentile plasma HGF levels, 75^{th} percentile plasma VEGF levels, 25^{th} percentile plasma s-VEGFR-3 levels, median Ang2 levels, median bFGF levels, and/or median IGF-2 levels in a population of HCC patients.
Aspect 13. The method according to aspect 12, wherein
said reference standard comprises ˜3.279 ng/ml plasma HGF levels, ˜101.928 pg/ml plasma VEGF levels ˜30.559 ng/ml plasma s-VEGFR-3 levels, ˜6.061 ng/ml plasma Ang2 levels, ˜7.5 pg/ml plasma bFGF levels, or 797.7 ng/ml plasma IGF-2 levels in a population of HCC patients.
Aspect 14. The method according to aspect 2, comprising detecting a combination of biomarkers, wherein said combination comprises

1) HGF and VEGF;

2) HGF and s-VEGFR-3;

3) VEGF and s-VEGFR-3;

4) HGF, VEGF and s-VEGFR-3;

5) HGF and Ras p21;

6) HGF, VEGF and Ras p21;

7) VEGF and Ras p21;

8) s-VEGFR-3 and Ras p21;

9) c-KIT and bFGF;

10) c-KIT and IGF-2;

11) bFGF and IGF-2;

12) HGF and bFGF; or

13) HGF and IGF-2;

14) any combination of a combination (1)-(13).

Aspect 15. The method according to aspect 2, comprising detecting at least one additional parameter which is

(a) Eastern Cooperative Oncology Group performance status (ECOG PS: 0 versus 1+2),

(b) macrovascular vascular invasion;

(c) tumor burden;

(d) extra-hepatic spread;

(e) levels of alpha fetoprotein (AFP);

(f) levels of alkaline phosphatase (AP);

(g) ascites;

(h) levels of bilirubin;

(i) levels of albumin;

(j) PT score; and/or

(k) child-pugh score.

Aspect 16. The method according to aspect 2, comprising detecting in a test sample of said patient, at least one biomarker which is plasma Ang2 and at least one additional parameter which is

(a) Eastern Cooperative Oncology Group performance status (ECOG PS: 0 versus 1+2),

(b) macrovascular vascular invasion;

(c) tumor burden;

(d) extra-hepatic spread;

(e) levels of alpha fetoprotein (AFP);

(f) levels of alkaline phosphatase (AP);

(g) ascites;

(h) levels of bilirubin;

(i) levels of albumin;

(j) PT score; and/or

(k) child-pugh score;

and comparing said plasma HGF levels and said additional parameter in said patient with

a reference standard; wherein

high levels of said plasma Ang2 levels combined with low levels of the additional parameter (i) or high levels of the additional parameter which is parameters (a)-(h) or parameter (j)-(k), is indicative of poor overall survival.

Aspect 17. The method according to aspect 2, comprising detecting in a test sample of said patient, at least one biomarker which is plasma HGF and at least one additional parameter which is

(a) macrovascular invasion,

(b) tumor burden,

(c) level of alpha fetoprotein (AFP),

(d) level of bilirubin,

(e) level of albumin and/or

(f) alkaline phosphatase (AP);

comparing said plasma HGF levels and said additional parameter in said patient with a reference standard; wherein

high levels of said plasma HGF combined with

low levels of the additional parameter (e) or high levels of the additional parameter which is parameters (a)-(d) or parameter (f),

is associated with poor overall survival.

Aspect 18. The method according to aspect 2, wherein said patient is treated with sorafenib.
Aspect 19. A method for predicting the outcome of sorafenib treatment in a patient suffering from HCC, comprising detecting, in a test sample of said patient, the expression levels of at least one biomarker which is vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin-2 (Ang2), basic fibroblast growth factor (bFGF) or insulin-like growth factor-2 (IGF-2) and comparing said levels to a reference standard, wherein differential expression of said biomarker in said test sample compared to said reference standard is indicative of said outcome of treatment.
Aspect 20. The method according to aspect 19, wherein said sorafenib comprises a compound of formula I below or a pharmaceutically acceptable salt, polymorph, hydrate, solvate thereof or a combination thereof.

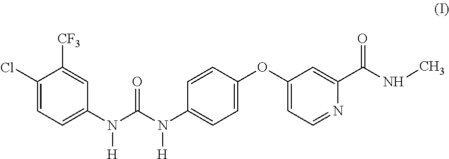

Aspect 21. The method according to aspect 19, wherein said sorafenib is N-[4-chloro-3-(trifluoromethyl)phenyl]-N′-{4-[2-carbamoyl-1-oxo-(4-pyridyloxy)]phenyl}urea or a tosylate salt thereof.

Aspect 22. The method according to aspect 19, wherein said c-KIT, HGF, Ras p21, s-VEGFR-2, and s-VEGFR-3 biomarkers are attenuated in said sorafenib-treated patients compared to said reference standard and/or VEGF levels are elevated in said sorafenib-treated patients compared to said reference standard.

Aspect 23. The method according to aspect 19, comprising detecting a combination of plasma biomarkers.
Aspect 24. The method according to aspect 23, wherein the combination comprises:

((a) Combinations comprising one biomarker from Group A and one biomarker from Group B

(i) HGF and VEGF;

(ii) s-c-Kit and VEGF;

(iii) s-VEGFR-3 and VEGF;

(iv) HGF and s-VEGFR-2;

(v) s-c-Kit and s-VEGFR-2;

(vi) s-VEGFR-3 and s-VEGFR-2;

(vii) Ang2 and VEGF;

(viii) Ang2 and sVEGFR2;

(ix) Ang2 and Ras p 21;

(x) IGF-2 and VEGF;

(xi) IGF-2 and sVEGFR2;

(xii) IGF-2 and Ras p21; or

(b) Combinations comprising one biomarker from Group A and two biomarkers from Group B

(i) HGF and VEGF plus s-VEGFR-2;

(ii) s-c-Kit and VEGF plus s-VEGFR-2;

(iii) s-VEGFR-3 and VEGF plus s-VEGFR-2;

(iv) Ang2 and VEGF plus sVEGFR2;

(v) Ang2 and sVEGFR2 plus Ras p21;

(vi) Ang2 and Ras p21 plus VEGF;

(vii) IGF-2 VEGF and sVEGFR2;

(viii) IGF-2, sVEGFR2 and Ras p21;

(ix) IGF-2, VEGF and Ras p21; or

(c) Combinations comprising two biomarkers from Group A and one biomarker from Group B

(i) HGF, s-c-Kit and VEGF;

(ii) HGF, s-c-Kit and s-VEGFR-2;

(iii) HGF, s-VEGFR-3 and VEGF;

(iv) HGF, s-VEGFR-3 and s-VEGFR-2;

(v) s-c-Kit, s-VEGFR-3 and VEGF;

(vi) s-c-Kit, s-VEGFR-3 and s-VEGFR-2;

(vii) HGF, Ang2 and VEGF;

(viii) HGF, Ang2 and s-VEGFR-2;

(ix) s-c-Kit, Ang2 and VEGF;

(x) s-c-Kit, s-VEGFR-3 and s-VEGFR-2;

(xi) s-VEGFR-3, Ang2 and VEGF;

(xii) s-VEGFR-3, Ang2 and s-VEGFR-2;

(xiii) IGF-2, HGF and VEGF;

(xiv) IGF-2, HGF and sVEGFR2;

(xv) IGF-2, HGF and Ras p21;

(xvi) IGF-2, Ang2 and VEGF;

(xvii) IGF-2, Ang2 and sVEGFR2;

(xviii) IGF-2, Ang2 and Ras p21;

(xix) IGF-2, s-c-Kit and VEGF;

(xx) IGF-2, s-c-Kit and sVEGFR2;

(xxi) IGF-2, s-c-Kit and Ras p21; or

(d) Combinations comprising two biomarkers from Group A and two biomarkers from Group B

(i) HGF, s-c-Kit and VEGF plus s-VEGFR-2;

(ii) HGF, s-VEGFR-3 and VEGF plus s-VEGFR-2;

(iii) s-c-Kit, s-VEGFR-3 and VEGF plus s-VEGFR-2;

(iv) HGF, Ang2 and VEGF plus s-VEGFR-2;

(v) s-c-Kit, Ang2 and VEGF plus s-VEGFR-2;

(vi) s-VEGFR-3, Ang2 and VEGF plus s-VEGFR-2;

(vii) IGF-2, HGF and VEGF plus sVEGFR2;

(viii) IGF-2, HGF and sVEGFR2 plus Ras p21;

(ix) IGF-2, HGF and VEGF plus Ras p21;

(x) IGF-2, Ang2 and VEGF plus sVEGFR2;

(xi) IGF-2, Ang2 and sVEGFR2 plus Ras p21;

(xii) IGF-2, Ang2 and VEGF plus Ras p21;

(xiii) IGF-2, s-c-Kit VEGF plus sVEGFR2;

(xiv) IGF-2, s-c-Kit and sVEGFR2 plus Ras p21;

(xv) IGF-2, s-c-Kit and VEGF plus Ras p21; or

(e) Combinations comprising three biomarkers from Group A and one biomarker from Group B

(i) HGF, s-c-Kit, s-VEGFR-3 and VEGF;

(ii) HGF, s-c-Kit, s-VEGFR-3 and s-VEGFR-2;

(iii) HGF, s-c-Kit, Ang2 and VEGF;

(iv) HGF, s-c-Kit, Ang2 and s-VEGFR-2;

(vi) s-c-Kit, s-VEGFR-3, Ang2 and VEGF;

(vi) s-c-Kit, s-VEGFR-3, Ang2 and s-VEGFR-2;

(vii) HGF, s-VEGFR-3, Ang2 and VEGF;

(viii) HGF, s-VEGFR-3, Ang2 and s-VEGFR-2;

(ix) HGF, s-c-Kit, IGF-2 and VEGF;

(x) HGF, s-c-Kit, IGF-2 and s-VEGFR-2;

(xi) HGF, IGF-2, Ang2 and VEGF;

(xii) HGF, IGF-2, Ang2 and s-VEGFR-2; or

(f) Combination comprising three biomarkers from Group A and two biomarkers from Group B

(i) HGF, s-c-Kit, s-VEGFR-3 and VEGF plus s-VEGFR-2;

(ii) HGF, s-c-Kit, Ang2 and VEGF plus s-VEGFR-2;

(iii) HGF, Ang2, s-VEGFR-3 and VEGF plus s-VEGFR-2;

(iv) s-c-Kit, s-VEGFR-3, Ang2 and VEGF plus s-VEGFR-2;

(v) HGF, s-c-Kit, IGF-2 and VEGF plus s-VEGFR-2;

(vi) HGF, s-c-Kit, IGF-2 and VEGF plus s-VEGFR-2;

(vii) HGF, IGF-2, Ang2 and VEGF plus s-VEGFR-2;

(viii) HGF, IGF-2, Ang2 and VEGF plus s-VEGFR-2; or

(g) Combination comprising four biomarkers from Group A and one biomarker from Group B

(i) HGF, s-c-Kit, s-VEGFR-3, Ang2 and VEGF;

(ii) HGF, s-c-Kit, s-VEGFR-3, Ang2 and s-VEGFR-2;

(iii) HGF, s-c-Kit, IGF-2, Ang2 and VEGF;

(iv) HGF, s-c-Kit, IGF-2, Ang2 and s-VEGFR-2; or

(h) Combination comprising four biomarkers from Group A and two biomarkers from Group B

(i) HGF, s-c-Kit, s-VEGFR-3, Ang2 and VEGF plus s-VEGFR-2;

(ii) HGF, s-c-Kit, IGF-2, Ang2 and VEGF plus s-VEGFR-2; or

(i) Combinations comprising all of the aforementioned biomarkers;
Aspect 25. The method according to aspect 19, wherein said outcome comprises evaluation of overall survival (OS), risk of death, time to progression (TTP), benefit of treatment (BOT), progression free survival (PFS), time to death (TTD), disease free survival (DFS), time to symptomatic progression (TSP), recurrence free survival (RFS), time to recurrence (TTR), disease state, response type, or a combination thereof.
Aspect 26. The method according to aspect 25, wherein said outcome comprises evaluation of overall survival (OS), risk of death, time to progression (TTP), benefit of treatment (BOT), or a combination thereof.
Aspect 27. A method for monitoring the response of an HCC patient towards sorafenib treatment comprising

detecting a baseline level of at least one biomarker which is s-c-Kit, HGF, Ras p21, VEGF, s-VEGFR-2, or s-VEGFR-3 in a test sample of said patient before sorafenib treatment,

detecting the level of said at least one biomarker in said test sample of said patient after sorafenib treatment, and

comparing said after sorafenib treatment biomarker level to said before sorafenib treatment baseline level,

wherein an attenuation in the levels of at least one of s-c-Kit, HGF, Ras p21, s-VEGFR-2, or s-VEGFR-3 and/or an elevation in the levels of VEGF in said test sample after sorafenib treatment is indicative that said patient is responsive to said sorafenib treatment.

Aspect 28. A method for evaluating the outcome of sorafenib treatment in a patient suffering from HCC, comprising

detecting the levels of plasma HGF in said patient at one time point;

detecting the levels of plasma HGF in said patient at a later time point; and

comparing said plasma HGF levels in said patient at the two time points;

wherein a reduction in said plasma HGF levels at said later time point is indicative of said outcome of sorafenib treatment.

Aspect 29. The method according to aspect 28, comprising

measuring plasma HGF levels before sorafenib treatment;

measuring plasma HGF levels at cycle 3 day 1 (C3D1);

determining the change in said plasma HGF levels; and

comparing said change with a reference value of 294 pg/mL plasma HGF, wherein a change in plasma HGF levels of >294 pg/mL at C3D1 indicates significantly longer time to progression.

Aspect 30. A method for prognosticating the outcome of a patient suffering from HCC, comprising

detecting, in a test tumor sample of said patient, the levels of phospho-ERK (pERK); and

comparing said levels of pERK with a reference standard;

wherein differential expression of said pERK in said tumor sample compared to a reference standard is indicative of the outcome of said HCC.

Aspect 31. The method according to aspect 30, wherein elevated levels of pERK in said tumor compared to said reference standard is indicative of longer TTP.
Aspect 32. The method according to aspect 30, wherein attenuated levels of pERK in said tumor compared to said reference standard is indicative of shorter TTP.
Aspect 33. A method of screening for an agent capable of influencing the outcome of patients with HCC, comprising

contacting a tumor cell to a test agent; and

detecting the expression level of at least one biomarker which is s-c-Kit, HGF, Ras p21, VEGF, s-VEGFR-2, s-VEGFR-3, or pERK before and after contacting with said agent;

wherein attenuation in the levels of s-c-Kit, HGF, Ras p21, s-VEGFR-2, or s-VEGFR-3 and/or elevation in the levels of VEGF or pERK after contacting with said agent indicates that said test agent is capable of influencing the outcome of said HCC.

Aspect 34. An antibody array or a kit which comprises of a plurality of antibody molecules, each of which specifically binds to an antigenic composition consisting of:
(a) Combinations comprising one biomarker from Group A and one biomarker from Group B

(i) HGF and VEGF;

(ii) s-c-Kit and VEGF;

(iii) s-VEGFR-3 and VEGF;

(iv) HGF and s-VEGFR-2;

(v) s-c-Kit and s-VEGFR-2;

(vi) s-VEGFR-3 and s-VEGFR-2;

(vii) Ang2 and VEGF;

(viii) Ang2 and sVEGFR2;

(ix) Ang2 and Ras p 21;

(x) IGF-2 and VEGF;

(xi) IGF-2 and sVEGFR2;

(xii) IGF-2 and Ras p21; or

(b) Combinations comprising one biomarker from Group A and two biomarkers from Group B

(i) HGF and VEGF plus s-VEGFR-2;

(ii) s-c-Kit and VEGF plus s-VEGFR-2;

(iii) s-VEGFR-3 and VEGF plus s-VEGFR-2;

(iv) Ang2 and VEGF plus sVEGFR2;

(v) Ang2 and sVEGFR2 plus Ras p21;

(vi) Ang2 and Ras p21 plus VEGF;

(vii) IGF-2 VEGF and sVEGFR2;

(viii) IGF-2, sVEGFR2 and Ras p21;

(ix) IGF-2, VEGF and Ras p21; or

(c) Combinations comprising two biomarkers from Group A and one biomarker from Group B

(i) HGF, s-c-Kit and VEGF;

(ii) HGF, s-c-Kit and s-VEGFR-2;

(iii) HGF, s-VEGFR-3 and VEGF;

(iv) HGF, s-VEGFR-3 and s-VEGFR-2;

(v) s-c-Kit, s-VEGFR-3 and VEGF;

(vi) s-c-Kit, s-VEGFR-3 and s-VEGFR-2;

(vii) HGF, Ang2 and VEGF;

(viii) HGF, Ang2 and s-VEGFR-2;

(ix) s-c-Kit, Ang2 and VEGF;

(x) s-c-Kit, s-VEGFR-3 and s-VEGFR-2;

(xi) s-VEGFR-3, Ang2 and VEGF;

(xii) s-VEGFR-3, Ang2 and s-VEGFR-2;

(xiii) IGF-2, HGF and VEGF;

(xiv) IGF-2, HGF and sVEGFR2;

(xv) IGF-2, HGF and Ras p21;

(xvi) IGF-2, Ang2 and VEGF;

(xvii) IGF-2, Ang2 and sVEGFR2;

(xviii) IGF-2, Ang2 and Ras p21;

(xix) IGF-2, s-c-Kit and VEGF;

(xx) IGF-2, s-c-Kit and sVEGFR2;

(xxi) IGF-2, s-c-Kit and Ras p21; or

(d) Combinations comprising two biomarkers from Group A and two biomarkers from Group B

(i) HGF, s-c-Kit and VEGF plus s-VEGFR-2;

(ii) HGF, s-VEGFR-3 and VEGF plus s-VEGFR-2;

(iii) s-c-Kit, s-VEGFR-3 and VEGF plus s-VEGFR-2;

(iv) HGF, Ang2 and VEGF plus s-VEGFR-2;

(v) s-c-Kit, Ang2 and VEGF plus s-VEGFR-2;

(vi) s-VEGFR-3, Ang2 and VEGF plus s-VEGFR-2;

(vii) IGF-2, HGF and VEGF plus sVEGFR2;

(viii) IGF-2, HGF and sVEGFR2 plus Ras p21;

(ix) IGF-2, HGF and VEGF plus Ras p21;

(x) IGF-2, Ang2 and VEGF plus sVEGFR2;

(xi) IGF-2, Ang2 and sVEGFR2 plus Ras p21;

(xii) IGF-2, Ang2 and VEGF plus Ras p21;

(xiii) IGF-2, s-c-Kit VEGF plus sVEGFR2;

(xiv) IGF-2, s-c-Kit and sVEGFR2 plus Ras p21;

(xv) IGF-2, s-c-Kit and VEGF plus Ras p21; or

(e) Combinations comprising three biomarkers from Group A and one biomarker from Group B

(i) HGF, s-c-Kit, s-VEGFR-3 and VEGF;

(ii) HGF, s-c-Kit, s-VEGFR-3 and s-VEGFR-2;

(iii) HGF, s-c-Kit, Ang2 and VEGF;

(iv) HGF, s-c-Kit, Ang2 and s-VEGFR-2;

(vi) s-c-Kit, s-VEGFR-3, Ang2 and VEGF;

(vi) s-c-Kit, s-VEGFR-3, Ang2 and s-VEGFR-2;

(vii) HGF, s-VEGFR-3, Ang2 and VEGF;

(viii) HGF, s-VEGFR-3, Ang2 and s-VEGFR-2;

(ix) HGF, s-c-Kit, IGF-2 and VEGF;

(x) HGF, s-c-Kit, IGF-2 and s-VEGFR-2;

(xi) HGF, IGF-2, Ang2 and VEGF;

(xii) HGF, IGF-2, Ang2 and s-VEGFR-2; or

(f) Combination comprising three biomarkers from Group A and two biomarkers from Group B

(i) HGF, s-c-Kit, s-VEGFR-3 and VEGF plus s-VEGFR-2;

(ii) HGF, s-c-Kit, Ang2 and VEGF plus s-VEGFR-2;

(iii) HGF, Ang2, s-VEGFR-3 and VEGF plus s-VEGFR-2;

(iv) s-c-Kit, s-VEGFR-3, Ang2 and VEGF plus s-VEGFR-2;

(v) HGF, s-c-Kit, IGF-2 and VEGF plus s-VEGFR-2;

(vi) HGF, s-c-Kit, IGF-2 and VEGF plus s-VEGFR-2;

(vii) HGF, IGF-2, Ang2 and VEGF plus s-VEGFR-2;

(viii) HGF, IGF-2, Ang2 and VEGF plus s-VEGFR-2; or

(g) Combination comprising four biomarkers from Group A and one biomarker from Group B

(i) HGF, s-c-Kit, s-VEGFR-3, Ang2 and VEGF;

(ii) HGF, s-c-Kit, s-VEGFR-3, Ang2 and s-VEGFR-2;

(iii) HGF, s-c-Kit, IGF-2, Ang2 and VEGF;

(iv) HGF, s-c-Kit, IGF-2, Ang2 and s-VEGFR-2; or

(h) Combination comprising four biomarkers from Group A and two biomarkers from Group B

(i) HGF, s-c-Kit, s-VEGFR-3, Ang2 and VEGF plus s-VEGFR-2;

(ii) HGF, s-c-Kit, IGF-2, Ang2 and VEGF plus s-VEGFR-2; or

(i) Combinations comprising all of the aforementioned biomarkers;
Aspect 35. An oligonucleotide array or a kit which comprises a plurality of oligonucleotide molecules, each of which specifically hybridize, under stringent hybridization conditions, with a combination consisting of the following genes:

(a) Combinations comprising one biomarker from Group A and one biomarker from Group B

(i) HGF and VEGF;

(ii) s-c-Kit and VEGF;

(iii) s-VEGFR-3 and VEGF;

(iv) HGF and s-VEGFR-2;

(v) s-c-Kit and s-VEGFR-2;

(vi) s-VEGFR-3 and s-VEGFR-2;

(vii) Ang2 and VEGF;

(viii) Ang2 and sVEGFR2;

(ix) Ang2 and Ras p 21;

(x) IGF-2 and VEGF;

(xi) IGF-2 and sVEGFR2;

(xii) IGF-2 and Ras p21; or

(b) Combinations comprising one biomarker from Group A and two biomarkers from Group B

(i) HGF and VEGF plus s-VEGFR-2;

(ii) s-c-Kit and VEGF plus s-VEGFR-2;

(iii) s-VEGFR-3 and VEGF plus s-VEGFR-2;

(iv) Ang2 and VEGF plus sVEGFR2;

(v) Ang2 and sVEGFR2 plus Ras p21;

(vi) Ang2 and Ras p21 plus VEGF;

(vii) IGF-2 VEGF and sVEGFR2;

(viii) IGF-2, sVEGFR2 and Ras p21;

(ix) IGF-2, VEGF and Ras p21; or

(c) Combinations comprising two biomarkers from Group A and one biomarker from Group B

(i) HGF, s-c-Kit and VEGF;

(ii) HGF, s-c-Kit and s-VEGFR-2;

(iii) HGF, s-VEGFR-3 and VEGF;

(iv) HGF, s-VEGFR-3 and s-VEGFR-2;

(v) s-c-Kit, s-VEGFR-3 and VEGF;

(vi) s-c-Kit, s-VEGFR-3 and s-VEGFR-2;

(vii) HGF, Ang2 and VEGF;

(viii) HGF, Ang2 and s-VEGFR-2;

(ix) s-c-Kit, Ang2 and VEGF;

(x) s-c-Kit, s-VEGFR-3 and s-VEGFR-2;

(xi) s-VEGFR-3, Ang2 and VEGF;

(xii) s-VEGFR-3, Ang2 and s-VEGFR-2;

(xiii) IGF-2, HGF and VEGF;

(xiv) IGF-2, HGF and sVEGFR2;

(xv) IGF-2, HGF and Ras p21;

(xvi) IGF-2, Ang2 and VEGF;

(xvii) IGF-2, Ang2 and sVEGFR2;

(xviii) IGF-2, Ang2 and Ras p21;

(xix) IGF-2, s-c-Kit and VEGF;

(xx) IGF-2, s-c-Kit and sVEGFR2;

(xxi) IGF-2, s-c-Kit and Ras p21; or

(d) Combinations comprising two biomarkers from Group A and two biomarkers from Group B

(i) HGF, s-c-Kit and VEGF plus s-VEGFR-2;

(ii) HGF, s-VEGFR-3 and VEGF plus s-VEGFR-2;

(iii) s-c-Kit, s-VEGFR-3 and VEGF plus s-VEGFR-2;

(iv) HGF, Ang2 and VEGF plus s-VEGFR-2;

(v) s-c-Kit, Ang2 and VEGF plus s-VEGFR-2;

(vi) s-VEGFR-3, Ang2 and VEGF plus s-VEGFR-2;

(vii) IGF-2, HGF and VEGF plus sVEGFR2;

(viii) IGF-2, HGF and sVEGFR2 plus Ras p21;

(ix) IGF-2, HGF and VEGF plus Ras p21;

(x) IGF-2, Ang2 and VEGF plus sVEGFR2;

(xi) IGF-2, Ang2 and sVEGFR2 plus Ras p21;

(xii) IGF-2, Ang2 and VEGF plus Ras p21;

(xiii) IGF-2, s-c-Kit VEGF plus sVEGFR2;

(xiv) IGF-2, s-c-Kit and sVEGFR2 plus Ras p21;

(xv) IGF-2, s-c-Kit and VEGF plus Ras p21; or

(e) Combinations comprising three biomarkers from Group A and one biomarker from Group B

(i) HGF, s-c-Kit, s-VEGFR-3 and VEGF;

(ii) HGF, s-c-Kit, s-VEGFR-3 and s-VEGFR-2;

(iii) HGF, s-c-Kit, Ang2 and VEGF;

(iv) HGF, s-c-Kit, Ang2 and s-VEGFR-2;

(vi) s-c-Kit, s-VEGFR-3, Ang2 and VEGF;

(vi) s-c-Kit, s-VEGFR-3, Ang2 and s-VEGFR-2;

(vii) HGF, s-VEGFR-3, Ang2 and VEGF;

(viii) HGF, s-VEGFR-3, Ang2 and s-VEGFR-2;

(ix) HGF, s-c-Kit, IGF-2 and VEGF;

(x) HGF, s-c-Kit, IGF-2 and s-VEGFR-2;

(xi) HGF, IGF-2, Ang2 and VEGF;

(xii) HGF, IGF-2, Ang2 and s-VEGFR-2; or

(f) Combination comprising three biomarkers from Group A and two biomarkers from Group B

(i) HGF, s-c-Kit, s-VEGFR-3 and VEGF plus s-VEGFR-2;

(ii) HGF, s-c-Kit, Ang2 and VEGF plus s-VEGFR-2;

(iii) HGF, Ang2, s-VEGFR-3 and VEGF plus s-VEGFR-2;

(iv) s-c-Kit, s-VEGFR-3, Ang2 and VEGF plus s-VEGFR-2;

(v) HGF, s-c-Kit, IGF-2 and VEGF plus s-VEGFR-2;

(vi) HGF, s-c-Kit, IGF-2 and VEGF plus s-VEGFR-2;

(vii) HGF, IGF-2, Ang2 and VEGF plus s-VEGFR-2;

(viii) HGF, IGF-2, Ang2 and VEGF plus s-VEGFR-2; or

(g) Combination comprising four biomarkers from Group A and one biomarker from Group B

(i) HGF, s-c-Kit, s-VEGFR-3, Ang2 and VEGF;

(ii) HGF, s-c-Kit, s-VEGFR-3, Ang2 and s-VEGFR-2;

(iii) HGF, s-c-Kit, IGF-2, Ang2 and VEGF;

(iv) HGF, s-c-Kit, IGF-2, Ang2 and s-VEGFR-2; or

(h) Combination comprising four biomarkers from Group A and two biomarkers from Group B

(i) HGF, s-c-Kit, s-VEGFR-3, Ang2 and VEGF plus s-VEGFR-2;

(ii) HGF, s-c-Kit, IGF-2, Ang2 and VEGF plus s-VEGFR-2; or

(i) an oligonucleotide array comprising all of the aforementioned biomarker genes.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1 show's association between plasma HGF levels and overall survival in patients with HCC. A p-value of 0.013 was observed in analysis of HGF as a continuous variable (not shown) and a p-value of 0.032 was observed in analysis of HGF as a binned variable (shown here). A 75^{th} percentile plasma HGF levels in HCC patients (3.279 ng/ml) was used as a reference standard for determination of “low” vs. “high” HGF levels.

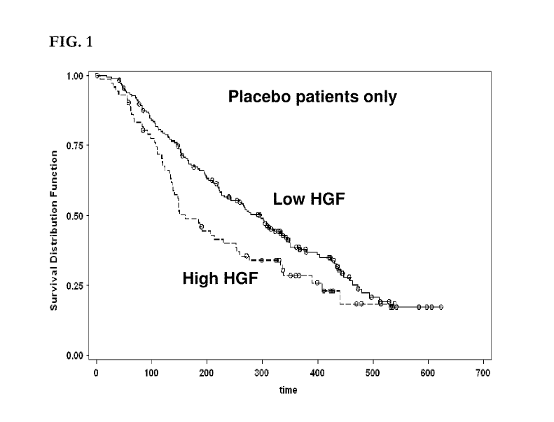

FIG. 2. shows association between plasma VEGF levels and overall survival in patients with HCC. A p-value of 0.001 was observed in analysis of VEGF as a continuous variable (not shown) and a p-value of 0.001 was observed in analysis of VEGF as a binned variable (shown here). A 75^{th} percentile plasma VEGF levels in HCC patients (101.928 pg/ml) was used as a reference standard for determination of “low” vs. “high” VEGF levels. A similar trend was observed for independently-assessed time to progression (TTP) (p=0.125).

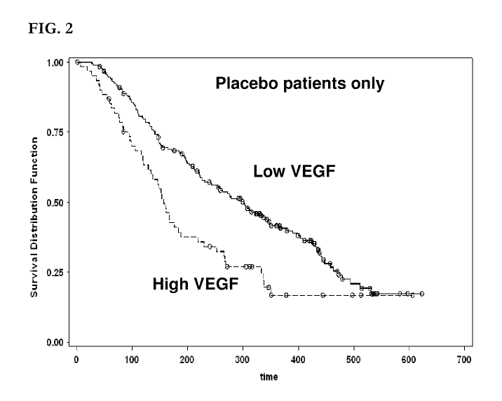

FIG. 3. shows association between plasma s-VEGFR-3 levels and overall survival in patients with HCC. A p-value of 0.014 was observed in analysis of s-VEGFR-3 as a continuous variable (not shown) and a p-value of 0.083 was observed in analysis of s-VEGFR-3 as a binned variable (shown here). A 25^{th} percentile plasma VEGFR-3 levels in HCC patients (30.559 ng/ml) was used as a reference standard for determination of “low” vs. “high” s-VEGFR-3 levels.

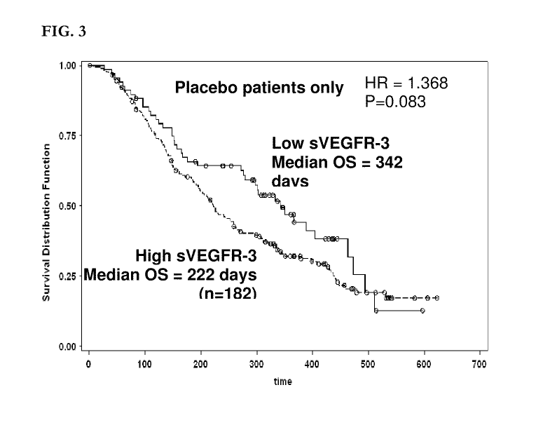

FIG. 4. shows association between plasma Ang2 levels and overall survival or time to progression in patients with HCC. A p-value of <0.0001 was observed in analysis of Ang2 as a as a prognostic indicator of OS and a p-value of 0.016 was observed in analysis of Ang2 as a prognostic indicator of TTP. A 50^{th} percentile plasma Ang2 levels in HCC patients (6.061 ng/ml) was used as a reference standard for determination of “low” vs. “high” Ang2 levels.

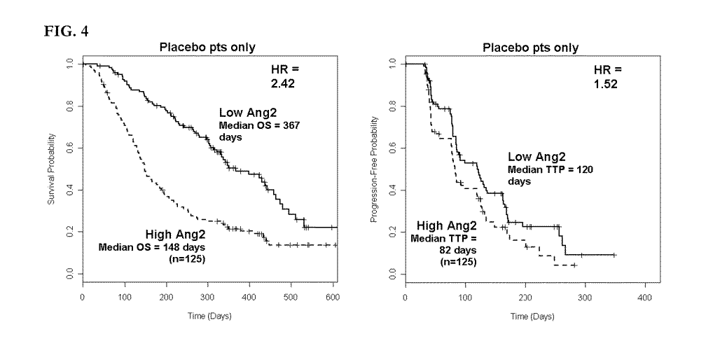

FIG. 5. shows association between plasma IGF-2 levels and overall survival in patients with HCC. A p-value of 0.002 was observed in analysis of IGF-2 as a as a prognostic indicator of OS. A 50^{th} percentile plasma IGF-2 levels in HCC patients (797.7 ng/ml) was used as a reference standard for determination of “low” vs. “high” IGF-2 levels.

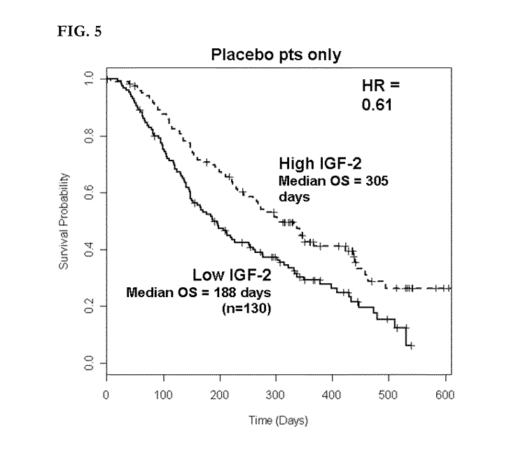

FIG. 6. shows correlation between Ang2 levels and VEGF levels. Weak correlation was observed between the two parameters in HCC patients.

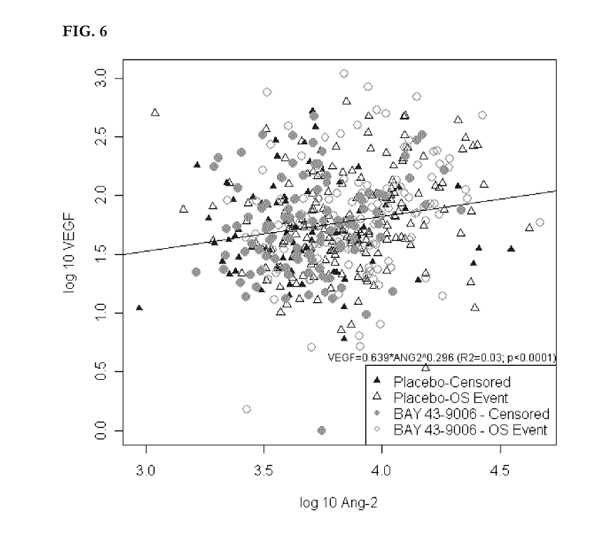

FIG. 7. shows the association between plasma s-c-Kit levels and sorafenib-mediated effect on overall survival in patients with HCC (p for interaction=0.081). A median plasma s-c-Kit levels in HCC patients (11.3 ng/ml) was used as a reference standard for determination of “low” vs. “high” s-c-Kit levels. A similar trend was seen with independently-assessed TTP (p for interaction=0.052) and with investigator-assessed TTP (p for interaction=0.117).

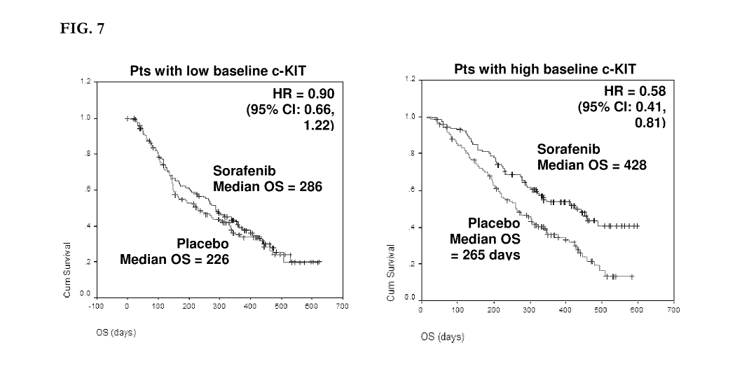

FIG. 8. shows association between plasma HGF levels and sorafenib-mediated effect on overall survival in patients with HCC (p for interaction=0.073). A 75^{th} percentile plasma HGF levels in HCC patients (3279.1 pg/ml) was used as a reference standard for determination of “low” vs. “high” HGF levels. A similar trend was observed with independently-assessed TTP (p for interaction=0.396) and with investigator-assessed TTP (p for interaction=0.246).

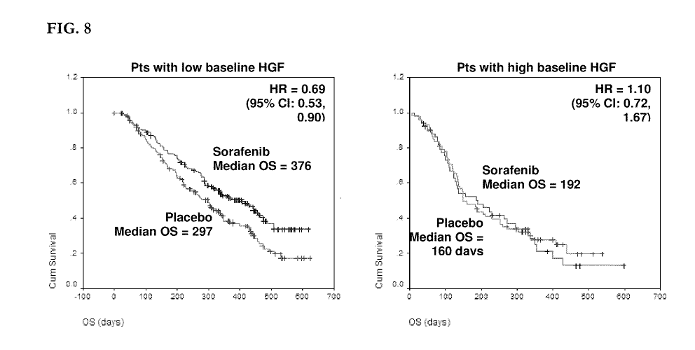

FIG. 9 shows association between plasma Ang2 levels and sorafenib-mediated effect on overall survival in patients with HCC (p for interaction=0.80). A 50^{th} percentile plasma HGF levels in HCC patients (6.061 ng/ml) was used as a reference standard for determination of “low” vs. “high” Ang2 levels.

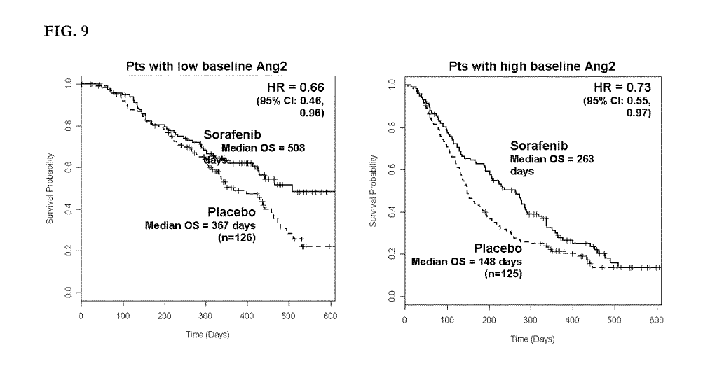

FIG. 10 shows association between plasma bFGF levels and sorafenib-mediated effect on time to progression in patients with HCC (p for interaction=0.078). A 50^{th} percentile plasma bFGF levels in HCC patients (7.5 pg/ml) was used as a reference standard for determination of “low” vs. “high” bFGF levels.

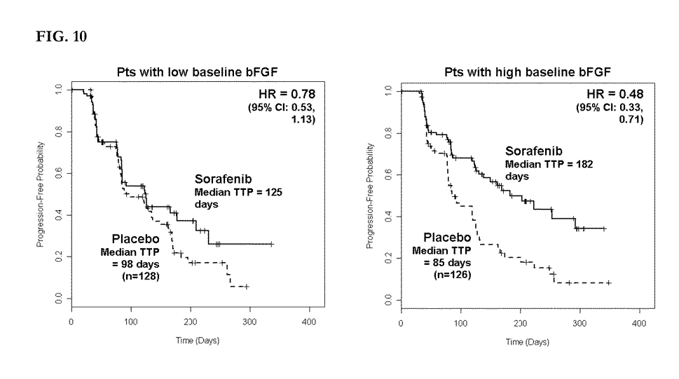

FIG. 11 shows association between plasma IGF-2 levels and sorafenib-mediated effect on time to progression in patients with HCC (p for interaction=0.13). A 50^{th} percentile plasma IGF-2 levels in HCC patients (797.7 ng/ml) was used as a reference standard for determination of “low” vs. “high” IGF-2 levels.

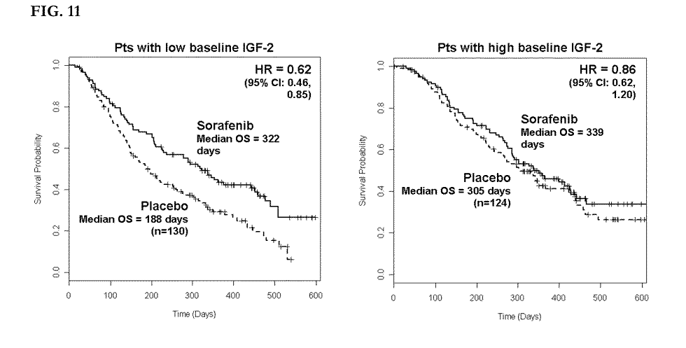

FIG. 12. shows C3D1 intra-patient changes in plasma s-c-Kit biomarker levels in HCC patients treated with sorafenib. Plasma c-KIT mean level decreases during sorafenib treatment and in the placebo group. However, the sorafenib-associated decrease is significantly different from the decrease observed in the placebo group (p<0.0001).

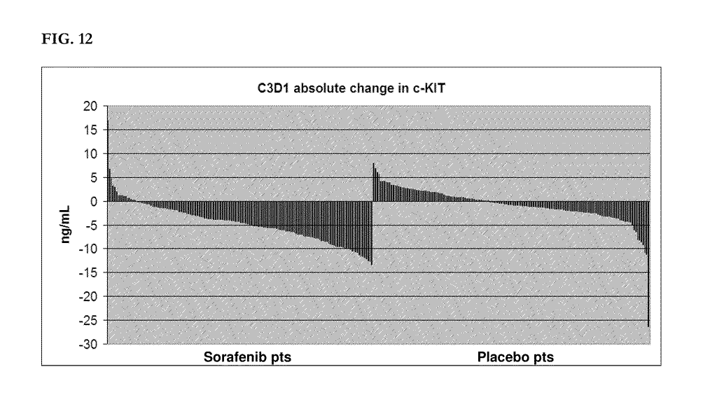

FIG. 13 shows C3D1 intra-patient changes in plasma HGF biomarker levels in HCC patients treated with sorafenib. Plasma HGF mean level decreases during sorafenib treatment while plasma HGF mean level increases in the placebo group. The sorafenib-associated decrease is significantly different from the increase observed in the placebo group (p<0.0001).

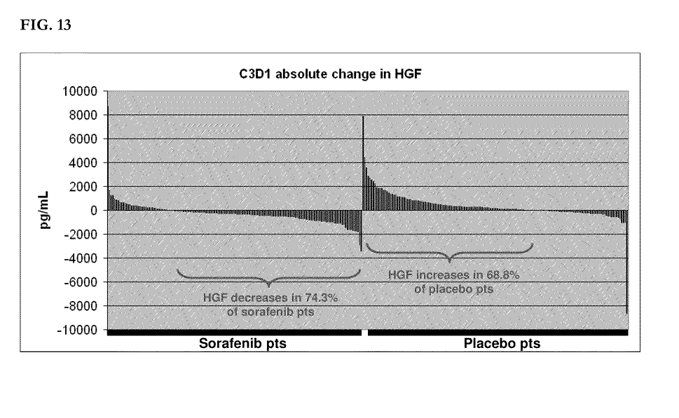

FIG. 14 illustrates mean intra-patient changes in s-c-Kit and HGF biomarker levels in HCC patients treated with sorafenib. Values at baseline and cycle 3 day 1 (C3D1) are shown. The sorafenib-associated decrease of both s-c-Kit and HGF is significantly different from the placebo group (p<0.0001).

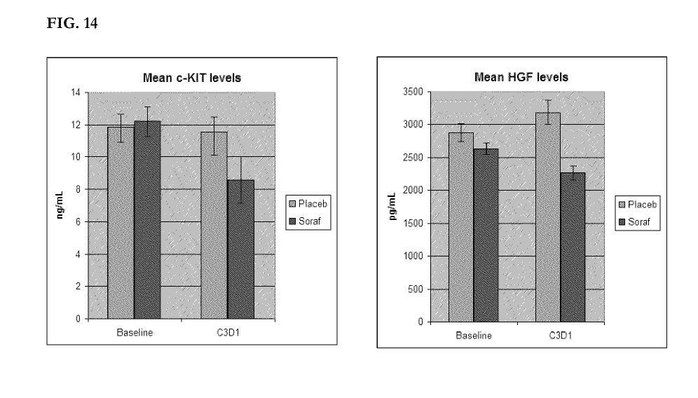

FIG. 15 illustrates mean intra-patient changes in Ras p21 and VEGF biomarker levels in HCC patients treated with sorafenib. Values at baseline and cycle 3 day 1 (C3D1) are shown. The sorafenib-associated decrease of Ras p21 is significantly different from the placebo group (p=0.046). The sorafenib-associated increase of VEGF is significantly different from the increase observed in the placebo group (p=0.010)

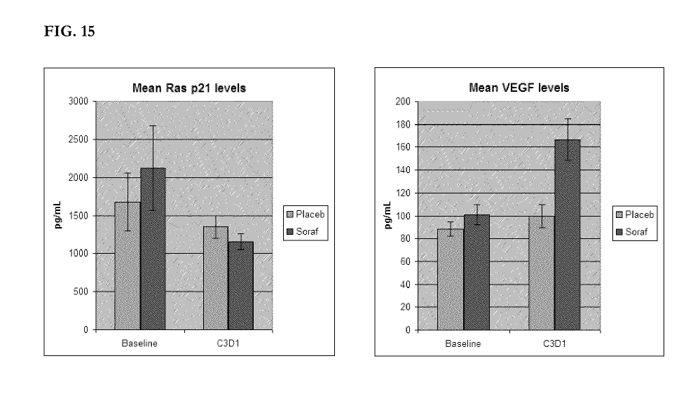

FIG. 16 illustrates changes in soluble VEGFR-2 and soluble VEGFR-3 biomarker levels in HCC patients treated with sorafenib. Values at baseline and cycle 3 day 1 (C3D1) are shown. The sorafenib-associated decrease of both biomarkers is significantly different from the placebo group (p<0.0001).

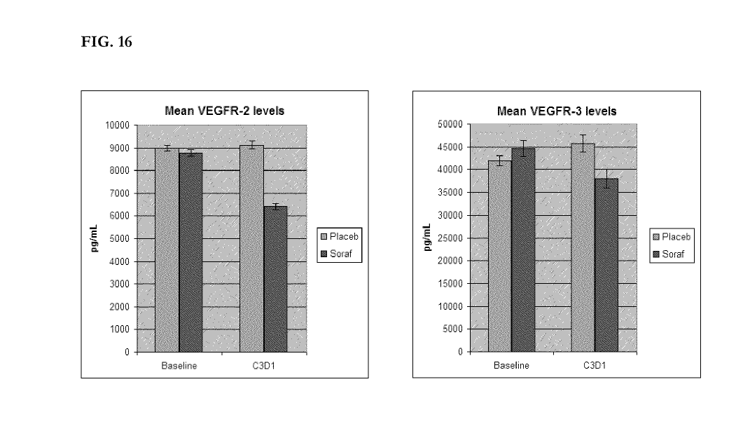

FIG. 17 shows C3D1 intra-patient changes in plasma Ang2 biomarker levels in HCC patients. The results show that Ang2 mean level increases in the placebo group, Ang2 mean level does not change significantly in the sorafenib group and the Ang2 change in the placebo group is significantly different from the sorafenib group (p<0.0001).

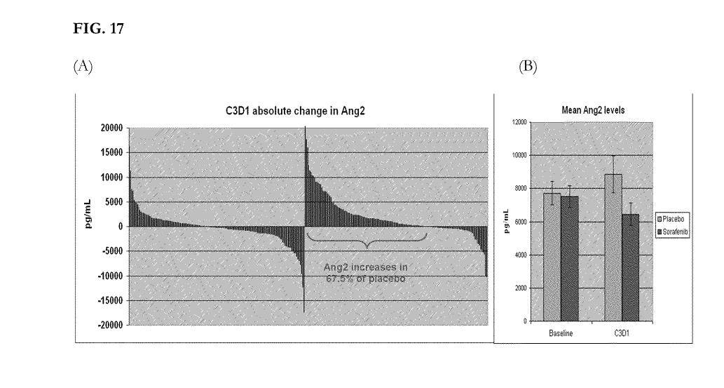

FIG. 18 shows C3D1 intra-patient changes in (A) plasma EGF and (B) plasma IGF-2 levels. Plasma EGF mean level decreases during sorafenib treatment (p=0.025*). Plasma IGF-2 mean level decreases during sorafenib treatment (p<0.0001*) and during placebo treatment (p<0.0001*).

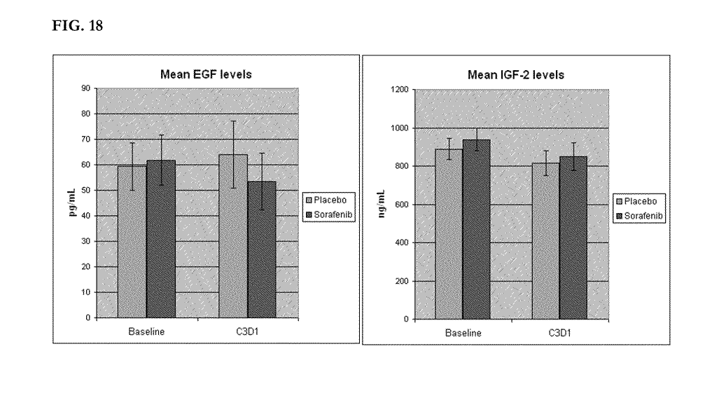

FIG. 19 shows intra-patient sorafenib-associated C3D1 change in plasma HGF (compared to baseline) in HCC patients. The median absolute change (decrease) of −294.02 pg/ml plasma HGF levels is shown.

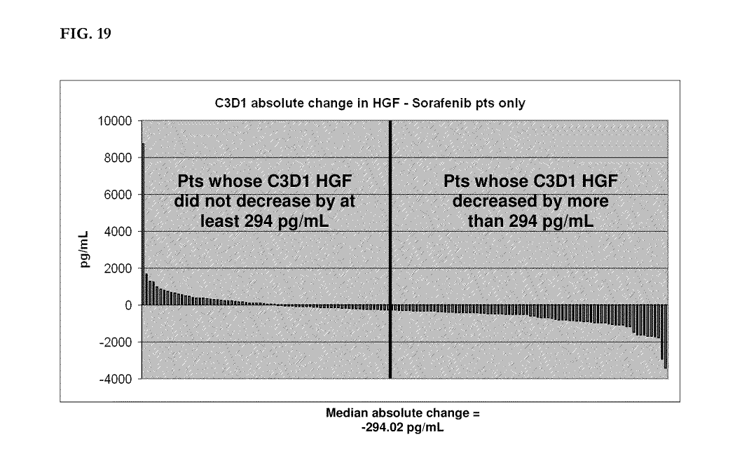

FIG. 20 shows association between sorafenib-associated C3D1 change in plasma HGF and outcome of treatment. Sorafenib patients whose plasma HGF levels decreased by more than 294.02 pg/mL (50^{th} percentile levels) at C3D1 (week 12) have longer time to progression (TTP) than patients whose plasma HGF levels did not decrease by similar amounts at this timepoint (p=0.029). This finding was consistently observed with (a) percentage change analysis of HGF versus independent TTP (p=0.083) (b) absolute change analysis of HGF versus investigator TTP (p=0.052); (c) percentage change analysis of HGF versus investigator TTP (p=0.016).

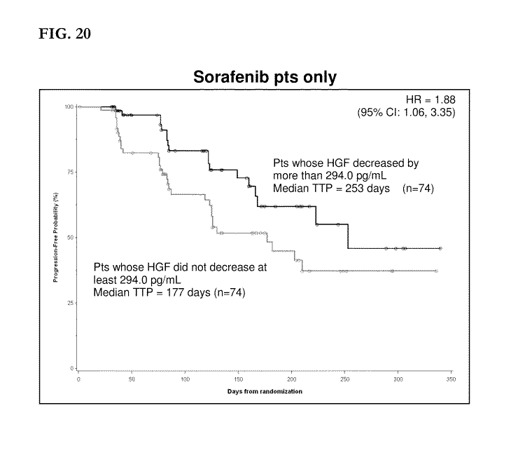

FIG. 21 shows intra-patient sorafenib-associated C3D1 change in plasma Ang2 levels (compared to baseline) in (A) sorafenib-treated HCC patients or (B) placebo-treated HCC patients. An increase in median Ang2 levels at C3D1 was observed in placebo patients.

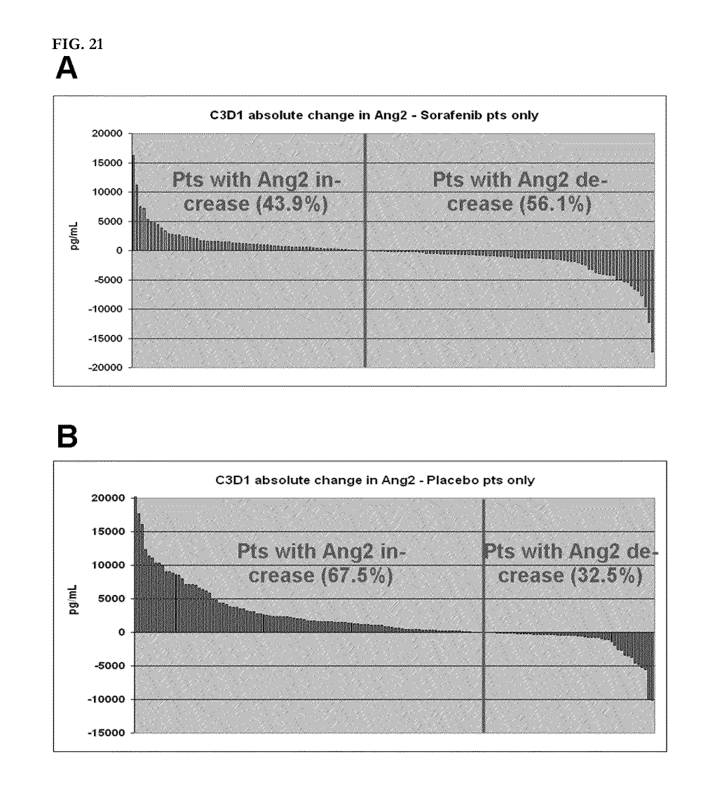

FIG. 22 shows association between change in plasma Ang2 levels at C3D1 and outcome of treatment. (A) Sorafenib-treated patients with Ang2 decrease have longer OS than patients with Ang2 increase (p<0.001). (B) Placebo patients with Ang2 decrease have longer OS than patients with Ang2 increase (p<0.0001).

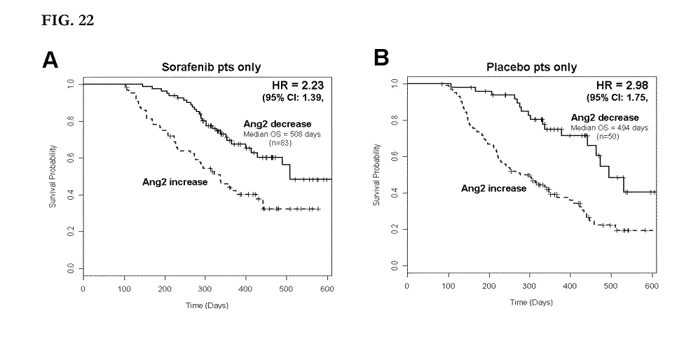

FIG. 23 shows association between change in plasma Ang2 levels at C3D1 and time to progression (TTP) outcome. Sorafenib-treated patients with Ang2 decrease have longer TTP than patients with Ang2 increase (p=0.005)

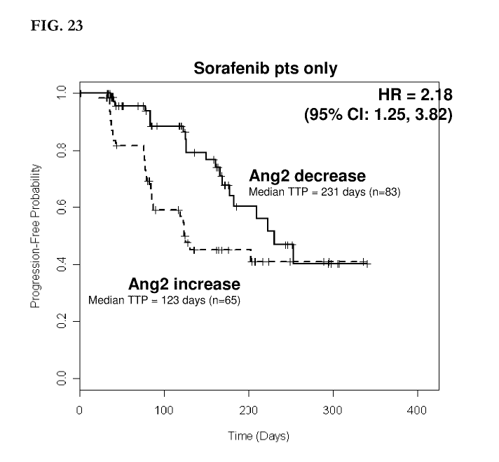

FIG. 24 shows intra-patient C3D1 change in plasma Ang2 levels (in terms of % change) in (A) sorafenib-treated/placebo HCC patients or (B) all HCC patients. An increase of 5.1% in median Ang2 levels at C3D1 was observed in placebo patients.

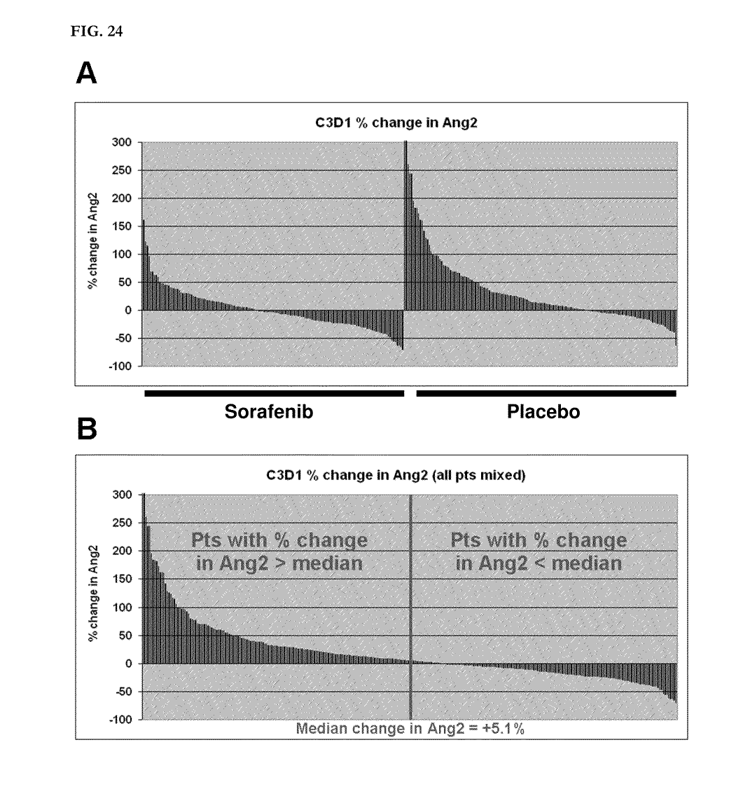

FIG. 25 shows association between percentage change in plasma Ang2 levels at C3D1 and outcome of treatment. (A) Sorafenib-treated patients with % change in Ang2 that is less than the median change of 5.1% have longer OS than patients with Ang2% change greater than the median change of 5.1% (p<0.001). (B) Placebo patients with % change in Ang2 less than the median change of 5.1% have longer OS than patients with Ang2% change greater than the median change of 5.1% (p<0.0001).

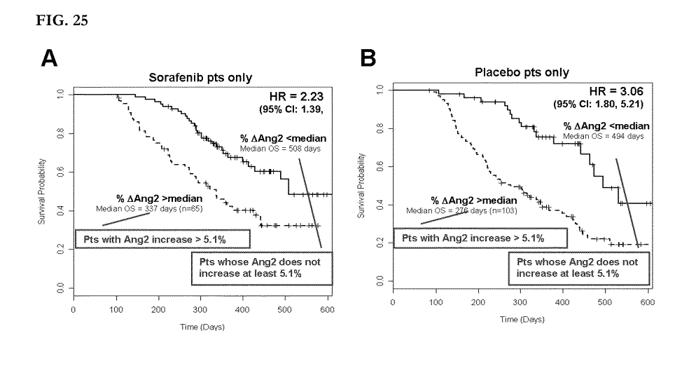

FIG. 26 shows absolute change in IGF2 at C3D1. A median reduction in IGF2 among all pts was observed (i.e., a reduction of −94.3 ng/mL).

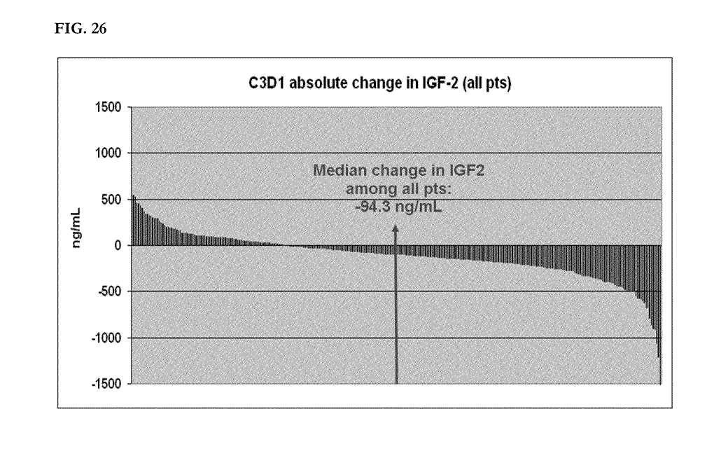

FIG. 27 shows association between change in plasma IGF-2 levels at C3D1 and outcome of treatment (OS). (A) Sorafenib-treated patients with IGF-2 change that is greater than the median reduction of 94.3 ng/mL have longer OS than patients with IGF-2 change that is lesser than the median reduction (p<0.011). (B) Placebo patients with IGF-2 change greater than reduction of 94.3 ng/mL have longer OS than patients with IGF-2 change that is lesser than the median reduction of 94.3 ng/mL (p=0.002).

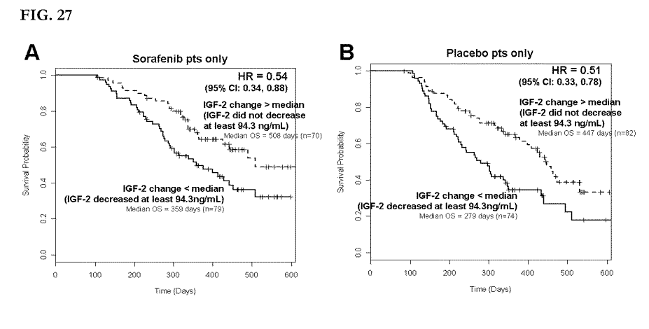

FIG. 28 shows association between change in plasma IGF-2 levels at C3D1 and outcome of treatment with respect to time to progression. Sorafenib-treated patients with IGF-2 change that is greater than the median reduction of 94.3 ng/mL have longer TTP than patients with IGF-2 change is lesser than the median reduction of 94.3 ng/mL (p=0.008).

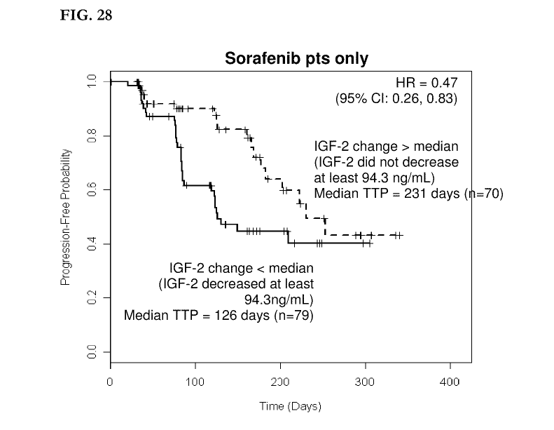

FIG. 29 shows intra-patient C3D1 change in plasma IGF-2 levels (in terms of % change) in (A) sorafenib-treated/placebo HCC patients or (B) all HCC patients. A decrease of 11.2% in median IGF-2 levels at C3D1 was observed in all patients.

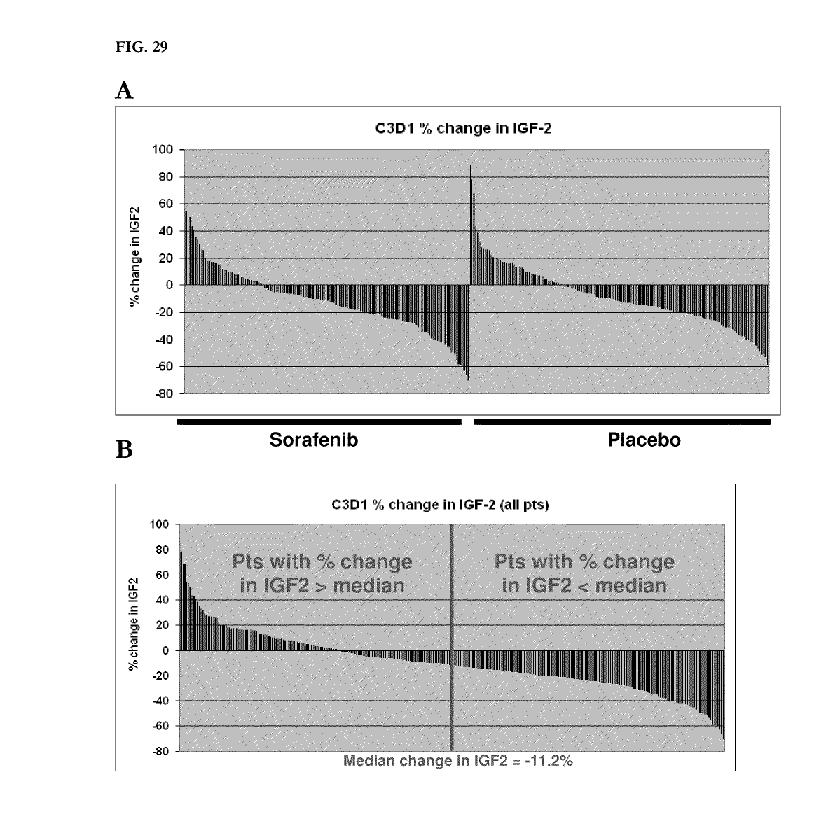

FIG. 30 shows association between percentage change in plasma IGF-2 levels at C3D1 and overall survival. (A) Sorafenib-treated patients with IGF-2 change that is greater than the median reduction of 11.2% have longer OS than patients with IGF-2 change that is lesser than the median reduction of 11.2% (p<0.063). The difference approached statistical significance. (B) Placebo patients with IGF-2 change greater than the median reduction of 11.2% have longer OS than patients with IGF-2 change that is lesser than the median reduction of 11.2% (p=0.0001).

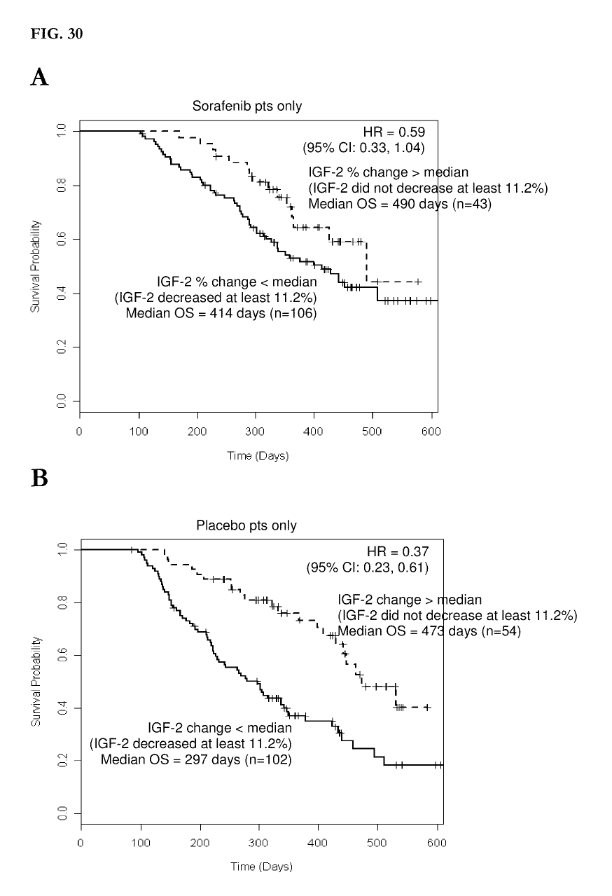

FIG. 31 shows Kaplan-Meier analysis of TTP based on baseline tumor pERK levels in HCC patients treated with sorafenib (N=33). Higher pre-treatment pERK levels (maximum tumor staining intensity) correlate significantly with longer TTP (Abou-Alfa et al, 2006, J. Clin Oncol.).

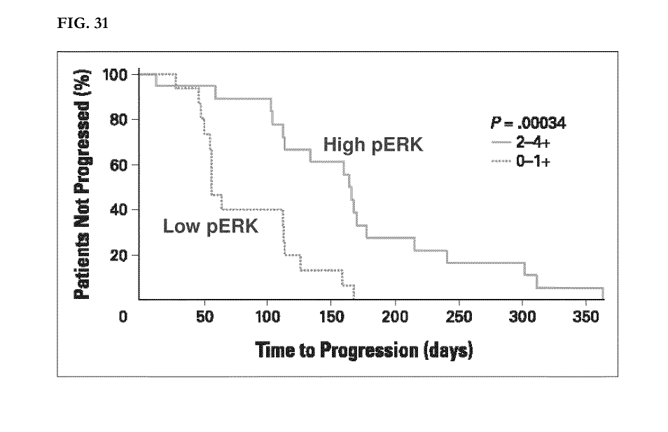

FIG. 32 shows overall survival (OS) and time to progression (TTP) in the polyclonal pERK subpopulation (N=107; 61 sorafenib treated, 46 placebo). HRs for OS and TTP in this subpopulation are representative of study population: Panel (A): p=0.104 for OS; Panel (B): p=0.0001 for independently-assessed TTP; and Panel (C): p=0.205 for investigator-assessed TTP.

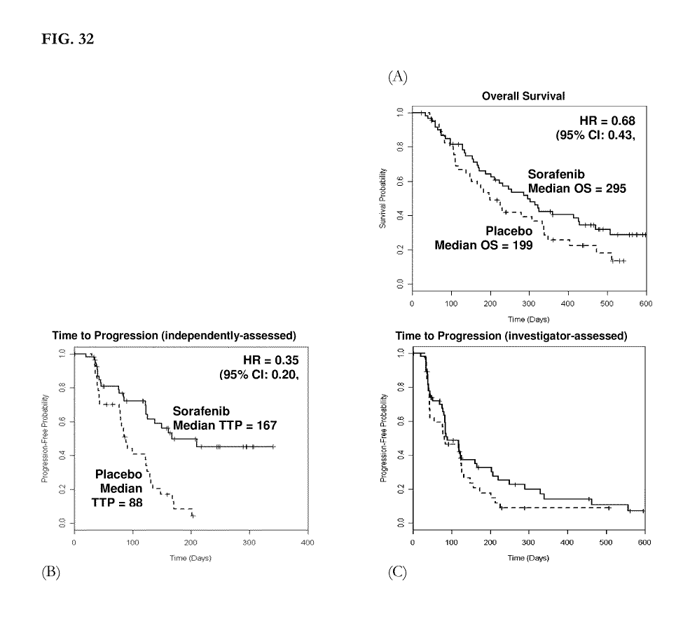

### EXAMPLES

The invention will be explained below with reference to the following non-limiting examples.

### Example 1

### Plasma ELISAs

Overview

Six candidate biomarker proteins were assayed by ELISA in patient plasma samples. These included VEGF, s-VEGFR-2, s-VEGFR-3, HGF, c-KIT, and Ras p21.

Plasma samples were obtained from patients at the time of screening, C3D1, and at the end of treatment visit. Samples from screening and C3D1 were assayed; samples from the end of treatment visit have not been assayed.

ELISA assays were performed on plasma samples from 512 patients. Listings of results by patient and timepoint are shown in Appendix 1.

ELISA Methods

All six assays were sandwich immunoassays obtained from commercial sources. All assays were performed according to the manufacturers' protocols, as summarized below. All samples were assayed in duplicate and the average value was used for correlative analysis. Averaged biomarker values for each timepoint for each patient are given in Appendix 1.

ELISAs for VEGF, s-VEGFR-2, HGF, and c-KIT

ELISA kits for VEGF (cat #DVE00), s-VEGFR-2 (cat #DVR200), HGF (cat #DHG00), and c-KIT (cat #DSCR00) were obtained from R&D Systems.

A monoclonal capture antibody specific to the target protein was provided pre-coated in microplate wells. Appropriately diluted samples and standards were pipetted into the wells, allowing capture of the target protein by the immobilized antibodies. Unbound sample was washed away, and a horseradish peroxidase-conjugated antibody also specific for the target protein was added to the wells. Wells were washed again, and a substrate solution was added to each well. The colored reaction product was measured spectrophotometrically and was translated to the quantity (pg/mL or ng/mL) of biomarker protein in the sample by use of a simultaneously generated standard curve.

ELISA for s-VEGFR-3

ELISA kits for s-VEGFR-3 (cat #DY349) were obtained from R&D Systems.

A capture antibody specific to the target protein was provided in solution by the manufacturer and was coated into microplate wells before running the assay. Appropriately diluted samples and standards were pipetted into the wells, allowing capture of the target protein by the immobilized antibodies. Unbound sample was washed away, and a biotinylated antibody also specific for the target protein was added to the wells. After washing, a streptavidin-horseradish peroxidase conjugate was added. Wells were washed again, followed by addition of a substrate solution. The colored reaction product was measured spectrophotometrically and was translated to the quantity (pg/mL) of biomarker protein in the sample by use of a simultaneously generated standard curve.

ELISA for Ras p21

ELISA kits for Ras p21 (cat #06490009) were from Oncogene Science Biomarker Group, part of Siemens Diagnostics, Cambridge, Mass. This assay detects all forms of circulating Ras p21 (H-Ras, N-Ras, and D-Ras).

A monoclonal capture antibody specific to the target protein was provided pre-coated in microplate wells. Appropriately diluted samples and standards were pipetted into the wells, allowing capture of the target protein by the immobilized antibodies. Unbound sample was washed away, and a biotinylated monoclonal antibody also specific for the target protein was added to the wells. After washing, a streptavidin-horseradish peroxidase conjugate was added. Wells were washed again, followed by addition of a substrate solution. The colored reaction product was measured spectrophotometrically and was translated to the quantity (pg/mL) of biomarker protein in the sample by use of a simultaneously generated standard curve.

Immunohistochemical Staining for Phosphorylated-ERK (pERK) in Biopsy Tumor

2.1 Overview

The goal of this correlative biomarker study was to examine the relationship of phosphorylated ERK (pERK) in archival, diagnostic tumor biopsies to the outcome of patient treatment with sorafenib in this placebo-controlled trial in HCC.

Immunohistochemistry (IHC) was performed on formalin-fixed paraffin-embedded (FFPE) diagnostic tumor biopsy samples received from hospitals participating in this trial and forwarded to Bayer Pharmaceuticals, West Haven, Conn. IHC was performed at 2 different CROs using 2 different anti-pERK antibodies. Oncotech performed IHC staining using a mouse monoclonal antibody from Sigma (cat #M8159), and Pathology Associates International (PAI), Frederick, Md. (a division of Charles River Laboratories) used a rabbit polyclonal antibody from Cell Signaling Technology (cat #CST 9101). Both antibodies were raised against ERK phosphorylated at Thr202/Tyr204. Staining procedures are described below. Stained slides were scored for pERK by pathologists provided by the CROs, and in some cases by consulting pathologist Dr David Rimm from Yale University. Pathologist scoring methods are described below.

FFPE samples were received from 143 patients, of which 125 were usable for pERK staining and analysis. Listings of the valid pERK results are shown in Appendix 1.

IHC Methods

Microtomy

FFPE samples were received from clinical sites as either paraffin blocks or already-mounted slides. Paraffin blocks were sectioned on a microtome at a thickness of 4 microns (Oncotech) or 5-6 microns (PAI). Sections were floated on a water bath and picked up onto glass microscope slides. Slides mounted with paraffin sections were then dried overnight prior to staining.

IHC Staining

Staining Procedure Using the Rabbit Polyclonal Antibody (CST #9101) at PAI

IHC staining at PAI was performed under GLP standards. An indirect standard ABC procedure (avidin-biotin-horseradish peroxidase complex) was used for the IHC staining of the clinical trial samples. The detection antibody was a rabbit polyclonal antibody to pERK (phospho-p44/42 MAPK (Thr202/Tyr204)) obtained from Cell Signaling Technology (cat #CST 9101). The negative control antibody was affinity purified anti-KLH (Keyhole Limpet Hemocyanin) [Rabbit], designated RbαKLH, directed against KLH, from Rockland. Secondary antibody was biotinylated goat anti-rabbit IgG. TBST+1% BSA served as the diluent for all antibodies.

1. Antigen retrieval and deparaffinization occurred by treating slides for 30 minutes with Declere solution in a pressure cooker at 95° C.
2. Slides were removed from the pressure cooker and allowed to reach room temperature for 30 minutes
3. Slides were rinsed 2× in deionized water
4. Slides were rinsed in Tris buffered saline, 0.15 M NaCl, pH 7.2)+0.05% Tween 20 (TBST) for 5 minutes
5. Slides were placed in a 1.5% hydrogen peroxide block for 10 minutes
6. Slides were rinsed 2× in TBST
7. Slides were then incubated for 30 minutes in a nonspecific protein block comprising 1% BSA and 1.5% normal goat serum in TBST
8. Detection and negative control antibodies (1:50 dilution) were applied for 30 minutes
9. Slides were rinsed 2× in TBST
10. Biotinylated secondary antibody was applied for 30 minutes
11. Slides were rinsed 2× in TBST
12. ABC Elite was applied to the slides for 30 minutes
13. Slides were rinsed 2× in TBST
14. Slides were treated with DAB substrate for 4 minutes
15. Slides were rinsed with tap water
16. Slides were counterstained with hematoxylin, followed by a wash
17. Slides were blued in saturated lithium carbonate, followed by a wash
18. Slides were dehydrated through alcohols, cleared in xylene, and coverslipped

### IHC Controls

For patients from whom a FFPE block was received, sections were cut and mounted, and an IgG negative control slide was stained for each sample. For patients from whom slides were received, an IgG negative control slide was not run due to the limited number of slides available.

In addition, positive control tissues were stained as part of each sample run. These controls consisted of (1+2) MIAPACA2 (human pancreatic adenocarcinoma) cell pellet blocks, either stimulated or unstimulated; (3+4) MDA-MB-231 (human breast carcinoma) cell pellet blocks, either stimulated or unstimulated; and 5) a mouse xenograft comprised of MDA-MB-231 (human breast carcinoma) cells. These controls were selected because in previous IHC experiments similar materials encompassed a range of pERK staining.

Staining Procedure Using the Mouse Monoclonal Antibody (Sigma #M8159) at Oncotech

The pERK IHC assay at Oncotech was designed and validated to be compatible with CLIA guidelines for a “homebrew” SRA class I test validation.

IHC staining for pERK was performed using the DAKO Mouse Envision Plus System (Cat #K4007) on the BioGenex i6000 Autostainer at room temperature. The detection antibody was a mouse monoclonal antibody to pERK (anti-MAP kinase, activated (diphosphorylated ERK-1&2), clone MAPK-YT, lot#015k4757) obtained from Sigma (cat #M8159). The negative control antibody was mouse IgG1 from DakoCytomation (lot#0018854). Secondary antibody was goat anti-mouse linked to horseradish peroxidase.

1. Antigen retrieval occurred by heating slides in BORG (Tris buffer, pH 9.5±0.2) at 120±3° C. for 3 min in a decloaking chamber
2. Slides were placed in the BioGenex i6000 Autostainer Plus chamber
3. Slides were rinsed once with wash buffer
4. Slides were incubated with Envision peroxidase for 5±1 minutes
5. Tissue sections were rinsed 2× with wash buffer
6. Slides were incubated with anti-pERK antibody (at 1.25 μg/ml) or the corresponding isotype negative reagent control (at the same concentration as the test article) for 30±1 minutes
7. Slides were rinsed once with wash buffer
8. Slides were incubated with goat anti-mouse polymer linked to horseradish peroxidase for 30±1 minutes
9. Slides were rinsed 2× with wash buffer
10. Slides were incubated with DAB Substrate for 5±1 minutes
11. Slides were again rinsed once with wash buffer
12. Slides were rinsed 2× with deionized water.
13. Stained slides were placed in a plastic slide basket submerged in deionized water
14. Slides were counterstained with hematoxylin
15. Slides were dehydrated through graded alcohols, cleared in xylene, and coverslipped

IHC Controls

For all patients with either a FFPE block or at least 2 slides available, an IgG negative control slide was stained for each sample.

In addition, a positive control tissue was stained as part of each sample run. FFPE breast cancer samples were provided by Oncotech for use as positive controls and test systems during testing. Specimens provided by Oncotech were collected in accordance with the IRB-approved ONC01 protocol.

For a subset of samples where the identity of the tissue as a tumor specimen was in question after pathologist examination of the slides, additional slides were stained with H&E to facilitate the pathologist's determination of tumor.

Pathologist Scoring

PAI Pathologist Scoring

Slides stained at PAI were scored by a PAI veterinary pathologist (Joan Wicks, DVM, PhD) and the scores were reviewed by a second PAI veterinary pathologist. (Note that slides stained at Oncotech using the monoclonal anti-pERK antibody were also sent to PAI for scoring.) The pathologists were blinded to patient identification, clinical outcome, and all relevant clinical data.

Semi-quantitative scoring evaluated staining intensity and percent of the tumor area stained for pERK. Intensity of staining was graded on the 5-point scale shown in Table 2-1. Note that intensity staining was reported as a range of observed intensities for a given sample (e.g. 2-4+). For statistical analysis of intensity data, the maximum staining intensity (the largest staining intensity in the range reported; in this example, 4+) was utilized. Percent area stained was graded on the scale shown in Table 2-2. Qualitative description of staining localization (nuclear (N) or cytoplasmic (C)) was also provided.

PAI Pathologist Scoring Scale for Staining Intensity

0=No cells staining
1+=Weak staining
2+=Moderate staining
3+=Strong staining
4+=Intense staining
Table 2-2: PAI pathology scoring scale for % tumor area stained
0=no cells staining
<5%=<5% cells
1st Quartile (1Q)=6-25% cells
2nd Quartile (2Q)=26-50% cells
3rd Quartile (3Q)=51-75% cells
4th Quartile (4Q)=76-100% cells

Oncotech Pathologist Scoring

Slides were scored by a medical pathologist. Slides stained using the monoclonal anti-pERK antibody were also sent to a consulting pathologist, for scoring. The pathologist was blinded to patient identification, clinical outcome, and all relevant clinical data.

Semi-quantitative scoring evaluated staining intensity and percent of the tumor area stained for pERK. Intensity of staining was graded on the 4-point scale shown. Note that the staining intensity scale are different. Tumor staining was reported in the format. This scoring detailed what percentage of cells stained positively for each level of staining intensity. A comprehensive score (H-score) was reported for tumor staining, which was calculated as follows:

H=(3×% cells staining 3+)+(2×% cells staining 2+)+(1×% cells staining 1+)

So, for the example of specimen 4, H=(3×0%)+(2×20%)+(1×0%)=40.

For statistical analysis of the scored tumor pERK data, two additional variables were derived from the pathology report: maximum staining intensity (the largest staining intensity at which any cells stained positive) and % cells stained positive (the sum of % cells stained at the 3+, 2+ and 1+ levels).

In addition to scoring tumor pERK staining, the pathologist provided a single intensity score for pERK staining in other cell types/structures.

Scoring by Consulting Pathologist

Slides stained at pathology lab using the monoclonal anti-pERK antibody were also sent to consulting pathologist for scoring. The consulting pathologist was blinded to patient identification, clinical outcome, and all relevant clinical data. The consulting pathologist developed a pERK scoring scale (0-2) designed with reproducibility in mind, which encompassed both staining intensity and area stained. The pathologist scored the stained samples on this scale for tumor cell pERK staining and for endothelial cell pERK staining.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions. All publications and patents cited above and in the following list are incorporated herein by reference.

Without further elaboration, it is believed that one skilled in the art can, using the preceding description, utilize the following invention to its fullest extent. The following specific preferred embodiments are, therefore, to be construed as merely illustrative, and not limitative of the remainder of the disclosure in any way whatsoever.

In the forgoing and in the following examples, all temperatures are set forth uncorrected in degrees Celsius and, all parts and percentages are by volume, unless otherwise indicated.

The entirety of the disclosure in the scientific abstract by J M Llovet, C Pena, M Shan, C Lathia and J Bruix et al. entitled “Plasma Biomarkers as Predictors of Outcome in Patients with Advanced Hepatocellular Carcinoma: Results from the Randomized Phase III SHARP Trial” (EASL Abstract, March 2009) which is appended to this application along with a copy of all the supplemental text, tables, and figure(s) associated with the manuscript, is incorporated herein by reference in its entirety.

We claim:

1. A method of prognosticating the outcome of sorafenib treatment of hepatocellular carcinoma (HCC) in a patient comprising
detecting at least one biomarker from vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin 2 (Ang2), basic fibroblast growth factor (bFGF), or insulin-like growth factor (IGF-2); and
comparing said level of expression of said biomarkers in said patient test sample with a reference standard,
wherein differential levels of expression of said biomarker in said test sample compared to said reference standard is indicative of said outcome of sorafenib treatment.

2. The method of prognosticating according to claim 1, wherein said outcome is time to progression, overall survival, benefit of treatment, or a combination thereof.

3. A method of prognosticating the outcome of a patient suffering from hepatocellular carcinoma (HCC), comprising
detecting, in a test sample of said patient, the expression levels of at least one biomarker which is vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin 2 (Ang2), basic fibroblast growth factor (bFGF) or insulin-like growth factor (IGF); and
comparing said level of expression of said biomarker in said patient test sample with a reference standard,
wherein differential levels of expression of said biomarker in said test sample compared to said reference standard is indicative of said outcome.

4. The method according to claim 3, wherein the biomarker is a protein.

5. The method according to claim 4, wherein said level of expression of said biomarker in said test sample is increased or decreased compared to said reference standard.

6. The method according to claim 4, wherein said biomarker is plasma HGF, VEGF, s-VEGFR-3, Ras p21, Ang2, bFGF, IGF-2 or a combination thereof.

7. The method according to claim 4, wherein said outcome is overall survival (OS) and/or time to progression (TTP).

8. The method according to claim 4, wherein said biomarker is HGF, VEGF, s-VEGFR-3, Ang2, IGF-2 and said outcome is overall survival (OS).

9. The method according to claim 8, wherein
attenuation of said HGF, VEGF, s-VEGFR-3, or Ang2 levels in said HCC patient compared to said reference standard; or
elevation of said IGF-2 in said HCC patient compared to said reference standard is indicative of improved overall survival (OS).

10. The method according to claim 8, wherein
elevation of said HGF, VEGF, s-VEGFR-3, or Ang2 levels in said HCC patient compared to said reference standard; or
attenuation of said IGF-2 in said HCC patient compared to said reference standard is indicative of worse overall survival.

11. The method according to claim 4, wherein said biomarker is VEGF, Ras p21, Ang2 and said outcome is time to progression (TTP).

12. The method according to claim 11, wherein attenuation of said VEGF levels, attenuation of said Ang2 levels, or elevation of Ras p21 levels in said HCC patient compared to said reference standard is indicative of longer time to progression (TTP).

13. The method according to claim 11, wherein elevation of said VEGF levels, elevation of said Ang2 levels, or attenuation of said Ras p21 levels in said HCC patient compared to said reference standard is indicative of shorter time to progression (TTP).

14. The method according to claim 6, wherein
said biomarker is plasma HGF, VEGF, s-VEGFR-3, Ang2, bFGF, or IGF-2; and
said reference standard comprises 75^{th} percentile plasma HGF levels, 75^{th} percentile plasma VEGF levels, 25^{th} percentile plasma s-VEGFR-3 levels, median Ang2 levels, median bFGF levels, and/or median IGF-2 levels in a population of HCC patients.

15. The method according to claim 14, wherein
said reference standard comprises ˜3.279 ng/ml plasma HGF levels, ˜101.928 pg/ml plasma VEGF levels ˜30.559 ng/ml plasma s-VEGFR-3 levels, ˜6.061 ng/ml plasma Ang2 levels, ˜7.5 pg/ml plasma bFGF levels, or 797.7 ng/ml plasma IGF-2 levels in a population of HCC patients.

16. The method according to claim 4, comprising detecting a combination of biomarkers, wherein said combination comprises
15) HGF and VEGF;
16) HGF and s-VEGFR-3;
17) VEGF and s-VEGFR-3;
18) HGF, VEGF and s-VEGFR-3;
19) HGF and Ras p21;
20) HGF, VEGF and Ras p21;
21) VEGF and Ras p21;
22) s-VEGFR-3 and Ras p21;
23) c-KIT and bFGF;
24) c-KIT and IGF-2;
25) bFGF and IGF-2;
26) HGF and bFGF; or
27) HGF and IGF-2;
28) any combination of a combination (1)-(13).

17. The method according to claim 4, comprising detecting at least one additional parameter which is
(a) Eastern Cooperative Oncology Group performance status (ECOG PS: 0 versus 1+2),
(b) macrovascular vascular invasion;
(c) tumor burden;
(d) extra-hepatic spread;
(e) levels of alpha fetoprotein (AFP);
(f) levels of alkaline phosphatase (AP);
(g) ascites;
(h) levels of bilirubin;
(i) levels of albumin;
(j) PT score; and/or
(k) child-pugh score.

18. The method according to claim 4, comprising detecting in a test sample of said patient, at least one biomarker which is plasma Ang2 and at least one additional parameter which is
(a) Eastern Cooperative Oncology Group performance status (ECOG PS: 0 versus 1+2),
(b) macrovascular vascular invasion;
(c) tumor burden;
(d) extra-hepatic spread;
(e) levels of alpha fetoprotein (AFP);
(f) levels of alkaline phosphatase (AP);
(g) ascites;
(h) levels of bilirubin;
(i) levels of albumin;
(j) PT score; and/or
(k) child-pugh score;
and comparing said plasma HGF levels and said additional parameter in said patient with
a reference standard; wherein
high levels of said plasma Ang2 levels combined with low levels of the additional parameter (i) or high levels of the additional parameter which is parameters (a)-(h) or parameter (j)-(k), is indicative of poor overall survival.

19. The method according to claim 4, comprising detecting in a test sample of said patient, at least one biomarker which is plasma HGF and at least one additional parameter which is
(a) macrovascular invasion,
(b) tumor burden,
(c) level of alpha fetoprotein (AFP),
(d) level of bilirubin,
(e) level of albumin and/or
(f) alkaline phosphatase (AP);
comparing said plasma HGF levels and said additional parameter in said patient with a reference standard; wherein
high levels of said plasma HGF combined with
low levels of the additional parameter (e) or high levels of the additional parameter which is parameters (a)-(d) or parameter (f),
is associated with poor overall survival.

20. The method according to claim 4, wherein said patient is treated with sorafenib.

21. A method for predicting the outcome of sorafenib treatment in a patient suffering from HCC, comprising detecting, in a test sample of said patient, the expression levels of at least one biomarker which is vascular endothelial growth factor (VEGF), soluble VEGF receptor 2 (s-VEGFR-2), soluble VEGF receptor 3 (VEGFR-3), soluble c-Kit (s-c-Kit), hepatocyte growth factor (HGF), Ras p 21, phosphorylated ERK (pERK), angiopoietin-2 (Ang2), basic fibroblast growth factor (bFGF) or insulin-like growth factor-2 (IGF-2) and comparing said levels to a reference standard, wherein differential expression of said biomarker in said test sample compared to said reference standard is indicative of said outcome of treatment.

22. The method according to claim 21, wherein said sorafenib comprises a compound of formula I below or a pharmaceutically acceptable salt, polymorph, hydrate, solvate thereof or a combination thereof.

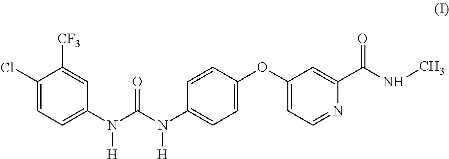

23. The method according to claim 21, wherein said sorafenib is N-[4-chloro-3-(trifluoromethyl)phenyl]-N′-{4-[2-carbamoyl-1-oxo-(4-pyridyloxy)]phenyl}urea or a tosylate salt thereof.

24. The method according to claim 21, wherein said c-KIT, HGF, Ras p21, s-VEGFR-2, and s-VEGFR-3 biomarkers are attenuated in said sorafenib-treated patients compared to said reference standard and/or VEGF levels are elevated in said sorafenib-treated patients compared to said reference standard.

25. The method according to claim 21, comprising detecting a combination of plasma biomarkers.

26. The method according to claim 25, wherein the combination comprises:
((a) Combinations comprising one biomarker from Group A and one biomarker from Group B
(i) HGF and VEGF;
(ii) s-c-Kit and VEGF;
(iii) s-VEGFR-3 and VEGF;
(iv) HGF and s-VEGFR-2;
(v) s-c-Kit and s-VEGFR-2;
(vi) s-VEGFR-3 and s-VEGFR-2;
(vii) Ang2 and VEGF;
(viii) Ang2 and sVEGFR2;
(ix) Ang2 and Ras p 21;
(x) IGF-2 and VEGF;
(xi) IGF-2 and sVEGFR2;
(xii) IGF-2 and Ras p21; or
(b) Combinations comprising one biomarker from Group A and two biomarkers from Group B
(i) HGF and VEGF plus s-VEGFR-2;
(ii) s-c-Kit and VEGF plus s-VEGFR-2;
(iii) s-VEGFR-3 and VEGF plus s-VEGFR-2;
(iv) Ang2 and VEGF plus sVEGFR2;
(v) Ang2 and sVEGFR2 plus Ras p21;
(vi) Ang2 and Ras p21 plus VEGF;
(vii) IGF-2 VEGF and sVEGFR2;
(viii) IGF-2, sVEGFR2 and Ras p21;
(ix) IGF-2, VEGF and Ras p21; or
(c) Combinations comprising two biomarkers from Group A and one biomarker from Group B
(i) HGF, s-c-Kit and VEGF;
(ii) HGF, s-c-Kit and s-VEGFR-2;
(iii) HGF, s-VEGFR-3 and VEGF;
(iv) HGF, s-VEGFR-3 and s-VEGFR-2;
(v) s-c-Kit, s-VEGFR-3 and VEGF;
(vi) s-c-Kit, s-VEGFR-3 and s-VEGFR-2;
(vii) HGF, Ang2 and VEGF;
(viii) HGF, Ang2 and s-VEGFR-2;
(ix) s-c-Kit, Ang2 and VEGF;
(x) s-c-Kit, s-VEGFR-3 and s-VEGFR-2;
(xi) s-VEGFR-3, Ang2 and VEGF;
(xii) s-VEGFR-3, Ang2 and s-VEGFR-2;
(xiii) IGF-2, HGF and VEGF;
(xiv) IGF-2, HGF and sVEGFR2;
(xv) IGF-2, HGF and Ras p21;
(xvi) IGF-2, Ang2 and VEGF;
(xvii) IGF-2, Ang2 and sVEGFR2;
(xviii) IGF-2, Ang2 and Ras p21;
(xix) IGF-2, s-c-Kit and VEGF;
(xx) IGF-2, s-c-Kit and sVEGFR2;
(xxi) IGF-2, s-c-Kit and Ras p21; or
(d) Combinations comprising two biomarkers from Group A and two biomarkers from Group B
(i) HGF, s-c-Kit and VEGF plus s-VEGFR-2;
(ii) HGF, s-VEGFR-3 and VEGF plus s-VEGFR-2;
(iii) s-c-Kit, s-VEGFR-3 and VEGF plus s-VEGFR-2;
(iv) HGF, Ang2 and VEGF plus s-VEGFR-2;
(v) s-c-Kit, Ang2 and VEGF plus s-VEGFR-2;
(vi) s-VEGFR-3, Ang2 and VEGF plus s-VEGFR-2;
(vii) IGF-2, HGF and VEGF plus sVEGFR2;
(viii) IGF-2, HGF and sVEGFR2 plus Ras p21;
(ix) IGF-2, HGF and VEGF plus Ras p21;
(x) IGF-2, Ang2 and VEGF plus sVEGFR2;
(xi) IGF-2, Ang2 and sVEGFR2 plus Ras p21;
(xii) IGF-2, Ang2 and VEGF plus Ras p21;
(xiii) IGF-2, s-c-Kit VEGF plus sVEGFR2;
(xiv) IGF-2, s-c-Kit and sVEGFR2 plus Ras p21;
(xv) IGF-2, s-c-Kit and VEGF plus Ras p21; or
(e) Combinations comprising three biomarkers from Group A and one biomarker from Group B
(i) HGF, s-c-Kit, s-VEGFR-3 and VEGF;
(ii) HGF, s-c-Kit, s-VEGFR-3 and s-VEGFR-2;
(iii) HGF, s-c-Kit, Ang2 and VEGF;
(iv) HGF, s-c-Kit, Ang2 and s-VEGFR-2;
(vi) s-c-Kit, s-VEGFR-3, Ang2 and VEGF;
(vi) s-c-Kit, s-VEGFR-3, Ang2 and s-VEGFR-2;
(vii) HGF, s-VEGFR-3, Ang2 and VEGF;
(viii) HGF, s-VEGFR-3, Ang2 and s-VEGFR-2;
(ix) HGF, s-c-Kit, IGF-2 and VEGF;
(x) HGF, s-c-Kit, IGF-2 and s-VEGFR-2;
(xi) HGF, IGF-2, Ang2 and VEGF;
(xii) HGF, IGF-2, Ang2 and s-VEGFR-2; or
(f) Combination comprising three biomarkers from Group A and two biomarkers from Group B
(i) HGF, s-c-Kit, s-VEGFR-3 and VEGF plus s-VEGFR-2;
(ii) HGF, s-c-Kit, Ang2 and VEGF plus s-VEGFR-2;
(iii) HGF, Ang2, s-VEGFR-3 and VEGF plus s-VEGFR-2;
(iv) s-c-Kit, s-VEGFR-3, Ang2 and VEGF plus s-VEGFR-2;
(v) HGF, s-c-Kit, IGF-2 and VEGF plus s-VEGFR-2;
(vi) HGF, s-c-Kit, IGF-2 and VEGF plus s-VEGFR-2;
(vii) HGF, IGF-2, Ang2 and VEGF plus s-VEGFR-2;
(viii) HGF, IGF-2, Ang2 and VEGF plus s-VEGFR-2; or
(g) Combination comprising four biomarkers from Group A and one biomarker from Group B
(i) HGF, s-c-Kit, s-VEGFR-3, Ang2 and VEGF;
(ii) HGF, s-c-Kit, s-VEGFR-3, Ang2 and s-VEGFR-2;
(iii) HGF, s-c-Kit, IGF-2, Ang2 and VEGF;
(iv) HGF, s-c-Kit, IGF-2, Ang2 and s-VEGFR-2; or
(h) Combination comprising four biomarkers from Group A and two biomarkers from Group B
(i) HGF, s-c-Kit, s-VEGFR-3, Ang2 and VEGF plus s-VEGFR-2;
(ii) HGF, s-c-Kit, IGF-2, Ang2 and VEGF plus s-VEGFR-2; or
(i) Combinations comprising all of the aforementioned biomarkers;

27. The method according to claim 21, wherein said outcome comprises evaluation of overall survival (OS), risk of death, time to progression (TTP), benefit of treatment (BOT), progression free survival (PFS), time to death (TTD), disease free survival (DFS), time to symptomatic progression (TSP), recurrence free survival (RFS), time to recurrence (TTR), disease state, response type, or a combination thereof.

28. The method according to claim 27, wherein said outcome comprises evaluation of overall survival (OS), risk of death, time to progression (TTP), benefit of treatment (BOT), or a combination thereof.

29. A method for monitoring the response of an HCC patient towards sorafenib treatment comprising
detecting a baseline level of at least one biomarker which is s-c-Kit, HGF, Ras p21, VEGF, s-VEGFR-2, or s-VEGFR-3 in a test sample of said patient before sorafenib treatment,
detecting the level of said at least one biomarker in said test sample of said patient after sorafenib treatment, and
comparing said after sorafenib treatment biomarker level to said before sorafenib treatment baseline level,
wherein an attenuation in the levels of at least one of s-c-Kit, HGF, Ras p21, s-VEGFR-2, or s-VEGFR-3 and/or an elevation in the levels of VEGF in said test sample after sorafenib treatment is indicative that said patient is responsive to said sorafenib treatment.

30. A method for evaluating the outcome of sorafenib treatment in a patient suffering from HCC, comprising
detecting the levels of plasma HGF in said patient at one time point;
detecting the levels of plasma HGF in said patient at a later time point; and
comparing said plasma HGF levels in said patient at the two time points;
wherein a reduction in said plasma HGF levels at said later time point is indicative of said outcome of sorafenib treatment.

31. The method according to claim 30, comprising
measuring plasma HGF levels before sorafenib treatment;
measuring plasma HGF levels at cycle 3 day 1 (C3D1);
determining the change in said plasma HGF levels; and
comparing said change with a reference value of 294 pg/mL plasma HGF, wherein a change in plasma HGF levels of >294 pg/mL at C3D1 indicates significantly longer time to progression.

32. A method for prognosticating the outcome of a patient suffering from HCC, comprising
detecting, in a test tumor sample of said patient, the levels of phospho-ERK (pERK); and
comparing said levels of pERK with a reference standard;
wherein differential expression of said pERK in said tumor sample compared to a reference standard is indicative of the outcome of said HCC.

33. The method according to claim 32, wherein elevated levels of pERK in said tumor compared to said reference standard is indicative of longer TTP.

34. The method according to claim 32, wherein attenuated levels of pERK in said tumor compared to said reference standard is indicative of shorter TTP.

35. A method of screening for an agent capable of influencing the outcome of patients with HCC, comprising
contacting a tumor cell to a test agent; and
detecting the expression level of at least one biomarker which is s-c-Kit, HGF, Ras p21, VEGF, s-VEGFR-2, s-VEGFR-3, or pERK before and after contacting with said agent;
wherein attenuation in the levels of s-c-Kit, HGF, Ras p21, s-VEGFR-2, or s-VEGFR-3 and/or elevation in the levels of VEGF or pERK after contacting with said agent indicates that said test agent is capable of influencing the outcome of said HCC.

36. An antibody array or a kit which comprises of a plurality of antibody molecules, each of which specifically binds to an antigenic composition consisting of:
(a) Combinations comprising one biomarker from Group A and one biomarker from Group B
(i) HGF and VEGF;
(ii) s-c-Kit and VEGF;
(iii) s-VEGFR-3 and VEGF;
(iv) HGF and s-VEGFR-2;
(v) s-c-Kit and s-VEGFR-2;
(vi) s-VEGFR-3 and s-VEGFR-2;
(vii) Ang2 and VEGF;
(viii) Ang2 and sVEGFR2;
(ix) Ang2 and Ras p 21;
(x) IGF-2 and VEGF;
(xi) IGF-2 and sVEGFR2;
(xii) IGF-2 and Ras p21; or
(b) Combinations comprising one biomarker from Group A and two biomarkers from Group B
(i) HGF and VEGF plus s-VEGFR-2;
(ii) s-c-Kit and VEGF plus s-VEGFR-2;
(iii) s-VEGFR-3 and VEGF plus s-VEGFR-2;
(iv) Ang2 and VEGF plus sVEGFR2;
(v) Ang2 and sVEGFR2 plus Ras p21;
(vi) Ang2 and Ras p21 plus VEGF;
(vii) IGF-2 VEGF and sVEGFR2;
(viii) IGF-2, sVEGFR2 and Ras p21;
(ix) IGF-2, VEGF and Ras p21; or
(c) Combinations comprising two biomarkers from Group A and one biomarker from Group B
(i) HGF, s-c-Kit and VEGF;
(ii) HGF, s-c-Kit and s-VEGFR-2;
(iii) HGF, s-VEGFR-3 and VEGF;
(iv) HGF, s-VEGFR-3 and s-VEGFR-2;
(v) s-c-Kit, s-VEGFR-3 and VEGF;
(vi) s-c-Kit, s-VEGFR-3 and s-VEGFR-2;
(vii) HGF, Ang2 and VEGF;
(viii) HGF, Ang2 and s-VEGFR-2;
(ix) s-c-Kit, Ang2 and VEGF;
(x) s-c-Kit, s-VEGFR-3 and s-VEGFR-2;
(xi) s-VEGFR-3, Ang2 and VEGF;
(xii) s-VEGFR-3, Ang2 and s-VEGFR-2;
(xiii) IGF-2, HGF and VEGF;
(xiv) IGF-2, HGF and sVEGFR2;
(xv) IGF-2, HGF and Ras p21;
(xvi) IGF-2, Ang2 and VEGF;
(xvii) IGF-2, Ang2 and sVEGFR2;
(xviii) IGF-2, Ang2 and Ras p21;
(xix) IGF-2, s-c-Kit and VEGF;
(xx) IGF-2, s-c-Kit and sVEGFR2;
(xxi) IGF-2, s-c-Kit and Ras p21; or
(d) Combinations comprising two biomarkers from Group A and two biomarkers from Group B
(i) HGF, s-c-Kit and VEGF plus s-VEGFR-2;
(ii) HGF, s-VEGFR-3 and VEGF plus s-VEGFR-2;
(iii) s-c-Kit, s-VEGFR-3 and VEGF plus s-VEGFR-2;
(iv) HGF, Ang2 and VEGF plus s-VEGFR-2;
(v) s-c-Kit, Ang2 and VEGF plus s-VEGFR-2;
(vi) s-VEGFR-3, Ang2 and VEGF plus s-VEGFR-2;
(vii) IGF-2, HGF and VEGF plus sVEGFR2;
(viii) IGF-2, HGF and sVEGFR2 plus Ras p21;
(ix) IGF-2, HGF and VEGF plus Ras p21;
(x) IGF-2, Ang2 and VEGF plus sVEGFR2;
(xi) IGF-2, Ang2 and sVEGFR2 plus Ras p21;
(xii) IGF-2, Ang2 and VEGF plus Ras p21;
(xiii) IGF-2, s-c-Kit VEGF plus sVEGFR2;
(xiv) IGF-2, s-c-Kit and sVEGFR2 plus Ras p21;
(xv) IGF-2, s-c-Kit and VEGF plus Ras p21; or
(e) Combinations comprising three biomarkers from Group A and one biomarker from Group B
(i) HGF, s-c-Kit, s-VEGFR-3 and VEGF;
(ii) HGF, s-c-Kit, s-VEGFR-3 and s-VEGFR-2;
(iii) HGF, s-c-Kit, Ang2 and VEGF;
(iv) HGF, s-c-Kit, Ang2 and s-VEGFR-2;
(vi) s-c-Kit, s-VEGFR-3, Ang2 and VEGF;
(vi) s-c-Kit, s-VEGFR-3, Ang2 and s-VEGFR-2;
(vii) HGF, s-VEGFR-3, Ang2 and VEGF;
(viii) HGF, s-VEGFR-3, Ang2 and s-VEGFR-2;
(ix) HGF, s-c-Kit, IGF-2 and VEGF;
(x) HGF, s-c-Kit, IGF-2 and s-VEGFR-2;
(xi) HGF, IGF-2, Ang2 and VEGF;
(xii) HGF, IGF-2, Ang2 and s-VEGFR-2; or
(f) Combination comprising three biomarkers from Group A and two biomarkers from Group B
(i) HGF, s-c-Kit, s-VEGFR-3 and VEGF plus s-VEGFR-2;
(ii) HGF, s-c-Kit, Ang2 and VEGF plus s-VEGFR-2;
(iii) HGF, Ang2, s-VEGFR-3 and VEGF plus s-VEGFR-2;
(iv) s-c-Kit, s-VEGFR-3, Ang2 and VEGF plus s-VEGFR-2;
(v) HGF, s-c-Kit, IGF-2 and VEGF plus s-VEGFR-2;
(vi) HGF, s-c-Kit, IGF-2 and VEGF plus s-VEGFR-2;
(vii) HGF, IGF-2, Ang2 and VEGF plus s-VEGFR-2;
(viii) HGF, IGF-2, Ang2 and VEGF plus s-VEGFR-2; or
(g) Combination comprising four biomarkers from Group A and one biomarker from Group B
(i) HGF, s-c-Kit, s-VEGFR-3, Ang2 and VEGF;
(ii) HGF, s-c-Kit, s-VEGFR-3, Ang2 and s-VEGFR-2;
(iii) HGF, s-c-Kit, IGF-2, Ang2 and VEGF;
(iv) HGF, s-c-Kit, IGF-2, Ang2 and s-VEGFR-2; or
(h) Combination comprising four biomarkers from Group A and two biomarkers from Group B
(i) HGF, s-c-Kit, s-VEGFR-3, Ang2 and VEGF plus s-VEGFR-2;
(ii) HGF, s-c-Kit, IGF-2, Ang2 and VEGF plus s-VEGFR-2; or
(i) Combinations comprising all of the aforementioned biomarkers;
Aspect 37. An oligonucleotide array or a kit which comprises a plurality of oligonucleotide molecules, each of which specifically hybridize, under stringent hybridization conditions, with a combination consisting of the following genes:
(a) Combinations comprising one biomarker from Group A and one biomarker from Group B
(i) HGF and VEGF;
(ii) s-c-Kit and VEGF;
(iii) s-VEGFR-3 and VEGF;
(iv) HGF and s-VEGFR-2;
(v) s-c-Kit and s-VEGFR-2;
(vi) s-VEGFR-3 and s-VEGFR-2;
(vii) Ang2 and VEGF;
(viii) Ang2 and sVEGFR2;
(ix) Ang2 and Ras p 21;
(x) IGF-2 and VEGF;
(xi) IGF-2 and sVEGFR2;
(xii) IGF-2 and Ras p21; or
(b) Combinations comprising one biomarker from Group A and two biomarkers from Group B
(i) HGF and VEGF plus s-VEGFR-2;
(ii) s-c-Kit and VEGF plus s-VEGFR-2;
(iii) s-VEGFR-3 and VEGF plus s-VEGFR-2;
(iv) Ang2 and VEGF plus sVEGFR2;
(v) Ang2 and sVEGFR2 plus Ras p21;
(vi) Ang2 and Ras p21 plus VEGF;
(vii) IGF-2 VEGF and sVEGFR2;
(viii) IGF-2, sVEGFR2 and Ras p21;
(ix) IGF-2, VEGF and Ras p21; or
(c) Combinations comprising two biomarkers from Group A and one biomarker from Group B
(i) HGF, s-c-Kit and VEGF;
(ii) HGF, s-c-Kit and s-VEGFR-2;
(iii) HGF, s-VEGFR-3 and VEGF;
(iv) HGF, s-VEGFR-3 and s-VEGFR-2;
(v) s-c-Kit, s-VEGFR-3 and VEGF;
(vi) s-c-Kit, s-VEGFR-3 and s-VEGFR-2;
(vii) HGF, Ang2 and VEGF;
(viii) HGF, Ang2 and s-VEGFR-2;
(ix) s-c-Kit, Ang2 and VEGF;
(x) s-c-Kit, s-VEGFR-3 and s-VEGFR-2;
(xi) s-VEGFR-3, Ang2 and VEGF;
(xii) s-VEGFR-3, Ang2 and s-VEGFR-2;
(xiii) IGF-2, HGF and VEGF;
(xiv) IGF-2, HGF and sVEGFR2;
(xv) IGF-2, HGF and Ras p21;
(xvi) IGF-2, Ang2 and VEGF;
(xvii) IGF-2, Ang2 and sVEGFR2;
(xviii) IGF-2, Ang2 and Ras p21;
(xix) IGF-2, s-c-Kit and VEGF;
(xx) IGF-2, s-c-Kit and sVEGFR2;
(xxi) IGF-2, s-c-Kit and Ras p21; or
(d) Combinations comprising two biomarkers from Group A and two biomarkers from Group B
(i) HGF, s-c-Kit and VEGF plus s-VEGFR-2;
(ii) HGF, s-VEGFR-3 and VEGF plus s-VEGFR-2;
(iii) s-c-Kit, s-VEGFR-3 and VEGF plus s-VEGFR-2;
(iv) HGF, Ang2 and VEGF plus s-VEGFR-2;
(v) s-c-Kit, Ang2 and VEGF plus s-VEGFR-2;
(vi) s-VEGFR-3, Ang2 and VEGF plus s-VEGFR-2;
(vii) IGF-2, HGF and VEGF plus sVEGFR2;
(viii) IGF-2, HGF and sVEGFR2 plus Ras p21;
(ix) IGF-2, HGF and VEGF plus Ras p21;
(x) IGF-2, Ang2 and VEGF plus sVEGFR2;
(xi) IGF-2, Ang2 and sVEGFR2 plus Ras p21;
(xii) IGF-2, Ang2 and VEGF plus Ras p21;
(xiii) IGF-2, s-c-Kit VEGF plus sVEGFR2;
(xiv) IGF-2, s-c-Kit and sVEGFR2 plus Ras p21;
(xv) IGF-2, s-c-Kit and VEGF plus Ras p21; or
(e) Combinations comprising three biomarkers from Group A and one biomarker from Group B
(i) HGF, s-c-Kit, s-VEGFR-3 and VEGF;
(ii) HGF, s-c-Kit, s-VEGFR-3 and s-VEGFR-2;
(iii) HGF, s-c-Kit, Ang2 and VEGF;
(iv) HGF, s-c-Kit, Ang2 and s-VEGFR-2;
(vi) s-c-Kit, s-VEGFR-3, Ang2 and VEGF;
(vi) s-c-Kit, s-VEGFR-3, Ang2 and s-VEGFR-2;
(vii) HGF, s-VEGFR-3, Ang2 and VEGF;
(viii) HGF, s-VEGFR-3, Ang2 and s-VEGFR-2;
(ix) HGF, s-c-Kit, IGF-2 and VEGF;
(x) HGF, s-c-Kit, IGF-2 and s-VEGFR-2;
(xi) HGF, IGF-2, Ang2 and VEGF;
(xii) HGF, IGF-2, Ang2 and s-VEGFR-2; or
(f) Combination comprising three biomarkers from Group A and two biomarkers from Group B
(i) HGF, s-c-Kit, s-VEGFR-3 and VEGF plus s-VEGFR-2;
(ii) HGF, s-c-Kit, Ang2 and VEGF plus s-VEGFR-2;
(iii) HGF, Ang2, s-VEGFR-3 and VEGF plus s-VEGFR-2;
(iv) s-c-Kit, s-VEGFR-3, Ang2 and VEGF plus s-VEGFR-2;
(v) HGF, s-c-Kit, IGF-2 and VEGF plus s-VEGFR-2;
(vi) HGF, s-c-Kit, IGF-2 and VEGF plus s-VEGFR-2;
(vii) HGF, IGF-2, Ang2 and VEGF plus s-VEGFR-2;
(viii) HGF, IGF-2, Ang2 and VEGF plus s-VEGFR-2; or
(g) Combination comprising four biomarkers from Group A and one biomarker from Group B
(i) HGF, s-c-Kit, s-VEGFR-3, Ang2 and VEGF;
(ii) HGF, s-c-Kit, s-VEGFR-3, Ang2 and s-VEGFR-2;
(iii) HGF, s-c-Kit, IGF-2, Ang2 and VEGF;
(iv) HGF, s-c-Kit, IGF-2, Ang2 and s-VEGFR-2; or
(h) Combination comprising four biomarkers from Group A and two biomarkers from Group B
(i) HGF, s-c-Kit, s-VEGFR-3, Ang2 and VEGF plus s-VEGFR-2;
(ii) HGF, s-c-Kit, IGF-2, Ang2 and VEGF plus s-VEGFR-2; or
(i) an oligonucleotide array comprising all of the aforementioned biomarker genes.
